(19) United States
(12) Patent Application Publication
(10) Pub. No.: US 2021/0009674 A1
(43) Pub. Date: Jan. 14, 2021

(54) BISPECIFIC ANTIBODIES TO TNF-ALPHA AND IL-1BETA AND USES THEREOF

(71) Applicant: Tavotek Biotherapeutics (Hong Kong) Limited, Hong Kong (CN)

(72) Inventors: Di ZHANG, Hillsborough, NJ (US); Susan H. TAM, Garnet Valley, PA (US); Minseon CHO, Breinigsville, PA (US); Motohiko NISHIDA, Ambler, PA (US); Lihua SHI, Wynnewood, PA (US); Man-Cheong FUNG, Ringoes, NJ (US)

(21) Appl. No.: 16/925,092

(22) Filed: Jul. 9, 2020

Related U.S. Application Data
(60) Provisional application No. 62/872,108, filed on Jul. 9, 2019.

(51) Int. Cl. C07K 16/24; A61K 39/395; A61K 45/06; A61P 19/02

(52) U.S. Cl. CPC C07K 16/241; A61K 2039/505; A61K 39/39541; A61K 45/06; A61P 19/02; C07K 2317/31; C07K 2317/76; C07K 2317/56; C07K 2317/52; C07K 2317/94; C07K 2317/53; C07K 2317/71; C07K 2317/524; C07K 2317/526; C07K 16/245

(57) ABSTRACT

The present disclosure relates to bi-specific antibodies that specifically bind and neutralize both tumor necrosis factor α (TNFα) and interleukin-1β (IL-1β), and to the use of such bispecific antibodies for the therapeutic treatment of TNFα and IL-1β-mediated diseases and disorders.

### CROSS REFERENCE TO RELATED APPLICATIONS

This application claims the benefit of priority to U.S. Provisional Application No. 62/872,108, filed on Jul. 9, 2019, which is herein incorporated by reference in its entirety.

### DESCRIPTION OF THE TEXT FILE SUBMITTED ELECTRONICALLY

The contents of the text file submitted electronically herewith are incorporated by reference in their entirety: A computer readable format copy of the Sequence Listing (filename: TABI_007_01US_SeqList_ST25.txt; date recorded Jul. 9, 2020; file size 147 kilobytes).

### BACKGROUND OF THE DISCLOSURE

It has been more than two decades since the first anti-TNF alpha (TNFα) monoclonal antibody (mAb) was approved to mitigate inflammation in patients with methotrexate-refractive rheumatoid arthritis (Mantzaris 2016, Moots, Curiale et al. 2018). Currently, there are several anti-TNFα monoclonal antibodies approved to treat inflammatory disorders. Despite successes in rheumatoid arthritis, inflammatory bowel diseases, and various auto-inflammatory disorders, there were well-documented risks associated with the use of anti-TNFα biologics (Taylor 2010). Besides infusion reactions, other serious adverse events such as thromboembolic events, lupus-like syndrome, vasculitis-like events and other autoimmune problems have been reported (Jani, Dixon et al. 2018). There were also increased infections, risks of increased lymphomas and other hematological malignancies, virus-caused cancers, congestive heart failure, and demyelinating events seen. For example, reactivation of tuberculosis, varicella-zoster (chickenpox), and herpes zoster (shingles) are commonly reported in patients receiving long term anti-TNFα therapy. Cases of exacerbated legionella have also been found along with reports of severe acute respiratory virus infections including new influenza and adenovirus infections. While the cause-association of some of these toxicities are not totally understood or established, caution in using anti-TNFα biologics in regard to these systemic safety issues is well recognized.

In view of the era of modern personalized medicine, developing novel agents with different potency and safety profile would allow better dose adjustments and optimal use of these therapies in patients with different inflammatory conditions. This is especially important because current anti-TNFα biologics infrequently bring complete and durable disease-free remission to patients despite initial responses. In fact, there are as much as one-third of patients treated by anti-TNFα biologics do not respond well (Owczarczyk-Saczonek, Owczarek et al. 2019). While the exact rationale is not totally clear, there points to the need of development of novel anti-TNFα or combination anti-cytokine therapy to address these challenges, especially to better identify and manage non-responders, develop more selective and effective anti-TNFα agent that block selective aspects of TNFR signaling, and better delivery of these agents to spare normal physiological effects of TNFα in non-diseased tissues. This disclosure addresses this and other needs.

### SUMMARY OF THE DISCLOSURE

The disclosure provides for bispecific antibodies and antigen-binding fragments thereof with dual specificity that specifically bind and neutralize, inhibit, block, abrogate, reduce, or interfere with both tumor necrosis factor alpha (TNFα) and interleukin 1β (IL-1β). The activity of TNFα and IL-1β that can be neutralized, inhibited, blocked, abrogated, reduced or interfered with, by the bispecific antibodies or fragments thereof of the disclosure, includes, but not by the way of limitation, neutralization of TNFα and IL-1β activation of their receptors, and the like.

As a non-limiting example, the disclosure provides for bispecific antibodies with dual specificity to both TNFα and IL-1β listed in Table 4 with combination of anti-TNFα antibodies listed in Table 2 and anti-IL-1β antibodies listed in Table 3 with different IgG Fc.

The disclosure provides for polynucleotides comprising the polynucleotide sequences encoding the bispecific antibodies with dual specificity to both TNFα and IL-1β listed in Table 4.

The disclosure also provides for monoclonal antibodies and antigen-binding fragments thereof that specifically bind and neutralize, inhibit, block, abrogate, reduce, or interfere with, at least one activity of tumor necrosis factor α (TNFα). The activity of TNFα that can be neutralized, inhibited, blocked, abrogated, reduced or interfered with, by the antibodies or fragments thereof of the disclosure, includes, but not by the way of limitation, neutralization of TNFα activation of its receptor, and the like.

As a non-limiting example, the disclosure provides for monoclonal anti-TNFα antibodies listed in Table 2 with different IgG Fc. The disclosure also provides for polynucleotides comprising the polynucleotide sequences encoding monoclonal anti-TNFα antibodies listed in Table 2.

The disclosure provides for monoclonal antibodies and antigen-binding fragments thereof that specifically bind and neutralize, inhibit, block, abrogate, reduce, or interfere with, at least one activity of human interleukin 1β (IL-1β). The activity of IL-1β that can be neutralized, inhibited, blocked, abrogated, reduced or interfered with, by the antibodies or fragments thereof of the disclosure, includes, but not by the way of limitation, neutralization of IL-1β activation of its receptor IL-1RI, and the like.

As a non-limiting example, the disclosure provides for monoclonal anti-IL-1β antibodies listed in Table 3 with different IgG Fc. The disclosure also provides for polynucleotides comprising the polynucleotide sequences encoding monoclonal anti-IL-1β antibodies listed in Table 3.

The disclosure also provides a method of generation bispecific antibody with dual specificity to both TNFα and IL-1β from two parental antibodies with F405L Fc mutation on one parental antibody and K409R Fc mutation on the other parental antibody by controlled Fab arm exchange.

As a non-limiting example, the disclosure provides a method of generation bispecific antibody with dual specificity to both TNFα and IL-1β listed in Table 4 with combination of anti-TNFα antibodies listed in Table 2 and anti-IL-1β antibodies listed in Table 3 with different IgG Fc by controlled Fab arm exchange.

The disclosure also provides for methods of detecting the formation of the anti-TNFα and IL-1β bispecific antibodies.

The anti-TNFα and anti-IL-1β monoclonal antibodies and bispecific antibodies can be full length IgG₁, IgG₂, IgG₃, IgG₄ antibodies or may comprise only an antigen-binding portion including a F_{ab}, F(_{ab′})₂, or scFv fragment. The antibody backbones may be modified to affect functionality, e.g., to eliminate residual effector functions.

The disclosure also provides for anti-TNFα and anti-IL-1β monoclonal antibodies and bispecific antibodies with an extended half-life when compared to the wild-type antibody. The extension of half-life can be realized by engineering the C_{H2} and C_{H3} domains of the antibody with any one set of mutations selected from M252Y/S254T/T256E, M428L/N434S, T250Q/M428L, N434A and T307A/E380A/N434A when compared to a parental wild-type antibody, residue numbering according to the EU Index.

The disclosure also provides for anti-TNFα and anti-IL-1β monoclonal antibodies and bispecific antibodies with enhanced resistant to proteolytic degradation by a protease that cleaves the wild-type antibody between or at residues 222-237 (EU numbering). The resistance to proteolytic degradation can be realized by engineering E233P/L234A/L235A mutations in the hinge region with G236 deleted when compared to a parental wild-type antibody, residue numbering according to the EU Index.

The disclosure also provides for vectors comprising the polynucleotides of the disclosure.

The disclosure also provides for a host cell comprising the vectors of the disclosure.

The disclosure also provides for a method of producing the anti-TNFα and anti-IL-1β monoclonal antibodies of the disclosure, comprising culturing the host cell of the disclosure under conditions that the antibody is expressed, and purifying the antibody.

The disclosure also provides for a pharmaceutical composition comprising the anti-TNFα and anti-IL-1β monoclonal antibodies and bispecific antibodies of the disclosure and a pharmaceutically acceptable carrier.

The disclosure also provides for methods of detecting the binding of the anti-TNFα and anti-IL-1β monoclonal antibodies and bispecific antibodies.

The disclosure also provides for methods of blocking the binding of TNFα and IL-1β to their receptors by the anti-TNFα and anti-IL-1β monoclonal antibodies and bispecific antibodies.

The disclosure also provides for methods of neutralizing the functional activity of TNFα and IL-1β to their receptors by the anti-TNFα and anti-IL-1β monoclonal antibodies and bispecific antibodies.

The disclosure also provides for methods of modulating the half-life of the anti-TNFα and anti-IL-1β monoclonal antibodies and bispecific antibodies.

The disclosure also provides for methods of modulating the resistance to proteolytic degradation of the anti-TNFα and anti-IL-1β monoclonal antibodies and bispecific antibodies.

The disclosure also provides for a method of treating auto-immune/inflammatory diseases. The disclosure also provides for use of the bispecific antibodies provided herein in a method of treating the auto-immune/inflammatory diseases; and for use of the bispecific antibodies provided herein in the manufacture of a medicament for use in the auto-immune/inflammatory diseases. Exemplary auto-immune and/or inflammatory diseases include, but are not limited to, the following: rheumatoid arthritis, systemic lupus erythematosus, osteoarthritis, ankylosing spondylitis, Behcet's Disease, gout, psoriatic arthritis, multiple sclerosis, Crohn's colitis, and inflammatory bowel disease, in a subject, comprising administering a therapeutically effective amount of bispecific antibodies with dual specificities to both TNFα and IL-113.

The disclosure also provides for a method of treating diabetes, nerve, eye, skin diseases. The disclosure also provides for use of the bispecific antibodies provided herein in a method of treating diabetes, nerve, eye, and skin diseases; and for use of the bispecific antibodies provided herein in the manufacture of a medicament for use in such diabetes, nerve, eye, and skin diseases. Exemplary diseases include but are not limited to: Type II diabetes mellitus, Parkinson's disease, age-related macular degeneration, polyneuropathy, sensory peripheral neuropathy, proliferative diabetic retinopathy, diabetic neuropathy, decubitus ulcer, fulminant Type 1 diabetes, retinal vasculitis, non-infectious posterior uveitis, alcoholic neuropathy, in a subject, comprising administering a therapeutically effective amount of bispecific antibodies with dual specificities to both TNFα and IL-113.

The disclosure also provides for a method of treating cancer. The disclosure also provides for use of the bispecific antibodies provided herein in a method of treating cancer; and for use of the bispecific antibodies provided herein in the manufacture of a medicament for use in cancer. Exemplary cancers include, but are not limited to: multiple myeloma, non-small cell lung cancer, acute myeloid leukemia, female breast cancer, pancreatic cancer, colorectal cancer and peritoneum cancer, in a subject, comprising administering a therapeutically effective amount of bispecific antibodies with dual specificities to both TNFα and IL-113. Modulating both TNFα and IL-1β may change the tumor microenvironment and the combination use of bispecific antibodies with dual specificities to both TNFα and IL-1β and antibodies to immune-oncology targets, such as PD1, may offer more effective therapeutic efficacies to treat cancers.

The disclosure also provides for a method of treating other diseases and inflammatory conditions which include but not limited to: chronic hepatitis B, leprosy, atrophic thyroiditis, small intestine enteropathy, sciatic neuropathy, and wound healing, in a subject, comprising administering a therapeutically effective amount of bispecific antibodies with dual specificities to both TNFα and IL-113. The disclosure also provides for use of the bispecific antibodies provided herein in a method of treating such other diseases and inflammatory conditions; and for use of the bispecific antibodies provided herein in the manufacture of a medicament for use in such other diseases and inflammatory disorders.

### BRIEF DESCRIPTION OF THE DRAWINGS

FIG. 1: Heavy chain and light chain amino acid sequences of anti-TNFα and IL-1β bispecific IgG1 antibody TAVO3334×5332.

FIG. 2: Heavy chain and light chain amino acid sequences of anti-TNFα IgG1 antibody TAVO3334.

FIG. 3: Heavy chain and light chain amino acid sequences of anti-IL-1β IgG1 antibody TAVO5332.

FIG. 4: Left two panels: SDS-PAGE analysis of anti-TNFα IgG1 antibody TAVO3334, anti-IL-1β IgG1 antibody TAVO5332 and anti-TNFα and IL-1β bispecific IgG1 antibody TAVO3334×5332. Right two panels: SDS-PAGE analysis of TAVO167127×14578, TAVO169127×14578, TAVO167128×14578, and TAVO169128×14578, which are anti-TNFα and IL-1β bispecific IgG1 antibodies engineered with E233P, L234A, L235A, F405L, M428L, N434S Fc mutations and with G236 deleted, and the corresponding parental antibodies TAVO167127, TAVO169127, TAVO167128, TAVO169128 and TAVO14578.

FIG. 5: Cation exchange chromatography profiles of anti-TNFα IgG1 antibody TAVO3334, anti-IL-1β IgG1 antibody TAVO5332 and anti-TNFα and IL-1β bispecific IgG1 antibody TAVO3334×5332 (left column) and anti-TNFα IgG1 antibody TAVO11934, anti-IL-113 IgG1 antibody TAVO12178 and anti-TNFα and IL-1β bispecific IgG1 antibody TAVO11934×12178 (right column).

FIG. 6: ELISA assays demonstrating the formation of anti-TNFα and IL-1β bispecific IgG1 antibody TAVO3334×5332 with dual binding to both TNFα and IL-1β.

FIG. 7: Binding to human, rhesus and mouse TNFα by anti-TNFα IgG1 antibody TAVO3334, anti-IL-1β IgG1 antibody TAVO5332 and anti-TNFα and IL-1β bispecific IgG1 antibody TAVO3334×5332.

FIG. 8: Binding to human, rhesus and mouse IL-1β by anti-TNFα IgG1 antibody TAVO3334, anti-IL-1β IgG1 antibody TAVO5332 and anti-TNFα and IL-1β bispecific IgG1 antibody TAVO3334×5332.

FIG. 9: Neutralizing human, rhesus and mouse TNFα cytotoxicity activity to WEHI cells by anti-TNFα IgG1 antibody TAVO3334, anti-IL-1β IgG1 antibody TAVO5332 and anti-TNFα and IL-1β bispecific IgG1 antibody TAVO3334×5332.

FIG. 10: Neutralizing human, rhesus and mouse IL-1β driven IL-6 release from activated MRC-5 cells by anti-TNFα IgG1 antibody TAVO3334, anti-IL-1β IgG1 antibody TAVO5332 and anti-TNFα and IL-1β bispecific IgG1 antibody TAVO3334×5332.

FIG. 11: Schematic of the principle of the HEK-Blue reporter assay for TNFα and IL-1β (left panel) and the response of reporter gene expression upon stimulation by TNFα, IL-1β and TNFα/IL-1β (right panel).

FIG. 12: Neutralizing TNFα, IL-1β and TNFα/IL-1β driven reporter gene activation by anti-TNFα IgG1 antibody TAVO3334, anti-IL-1β IgG1 antibody TAVO5332 and anti-TNFα and IL-1β bispecific IgG1 antibody TAVO3334×5332 in HEK-Blue reporter assays.

FIG. 13: Neutralizing TNFα/IL-1β driven reporter gene activation by anti-TNFα and IL-113 bispecific antibodies TAVO3334×7378, TAVO11934×12032, TAVO11934×12178, TAVO14434×14578, TAVO167127×14578, TAVO169127×14578, TAVO167128×14578, and TAVO169128×14578 in HEK-Blue reporter assays.

FIG. 14: Binding to mouse FcRn at pH 6.0 by anti-TNFα and IL-1β bispecific antibodies TAVO11934×12032 and TAVO11934×12178 with half-life extension F_{c} mutations and TAVO3334×5332 and TAVO3334×7378 lacking such mutations.

FIG. 15: SDS-PAGE analysis of the integrity of heavy chains for anti-TNFα and IL-1β bispecific antibody TAVO14434×14578 and its parental antibodies TAVO14434 and TAVO14578 with proteolytic degradation resistant F_{c} mutations and TAVO3334×7378 and TAVO11934×12178 lacking such mutations after digestion by IgG protease IdeZ and Matrix Metalloproteinase 3 (MMP3).

FIG. 16: Effect of anti-TNFα and IL-1β bispecific IgG1 antibody TAVO3334×5332 in inhibiting arthritic phenotype in a CAIA model using Tg1278/TNFKO mice. Left panel: The effect of the tested compounds on the arthritic score of experimental Tg1278/TNFKO mice. By the end of the study, the mean arthritis disease severity scores in the treatment groups were as follows: PBS=9.8±1.0, TAVO3334×5332 1 mg/kg=8.1±1.1, TAVO3334×5332 5 mg/kg=6.6±0.9, and TAVO3334×5332 10 mg/kg=3.5±0.5; Right panel: The effect of the tested compounds on the mean body weight of Tg1278/TNFKO mice. Mean body weights in the treatment groups were as follows: PBS=21.7±0.2 g, TAVO3334×5332 1 mg/kg=22.8±0.8 g, TAVO3334×5332 5 mg/kg=23.5±0.06 g, and TAVO3334×5332 10 mg/kg=23.1±0.8 g. Error bars indicate the standard error of the mean.

FIGS. 17A and 17B: Knee joint swelling induced by intra-articular injection of NIH3T3 cells expressing either human TNFα or human IL-1β into the knee joint of DBA-1 mice. 1×10⁴, 5×10⁴, or 25×10⁴ of NIH3T3: hTNFα cells or NIH3T3: hIL-1β cells were injected into the right knee of male DBA-1 mice of 9-10 weeks old, while the left knee was injected with equivalent numbers of NIH3T3 parental cells. Caliper measurements of both knee joints were conducted each day after cell injection for three days. Change in joint swelling was expressed as the mean difference between the right treated knee and the left control knee as measured by caliper for NIH3T3: hTNFα cells (FIG. 17A) or NIH3T3: hIL-1β cells (FIG. 17B).

FIGS. 18A, 18B, and 18C: Suppression of knee joint swelling by anti-TNFα and IL-1β bispecific antibody TAVO11934×12178 and its associated parental antibodies in normal mice. Male DBA-1 mice were dosed intraperitoneally on Day 0 with 10 mg/kg anti-TNFα and IL-1β bispecific antibody TAVO11934×12178, a mixture of 5 mg/kg anti-TNFα antibody TAVO11934 and 5 mg/kg isotype control antibody, a mixture of 5 mg/kg anti-IL-1β antibody TAVO12178 and 5 mg/kg isotype control antibody, or 10 mg/kg isotype control antibody 2 hours prior to intra-articular injection of an inflammatory cell mixture into the right knee or control cells into the left knee. Inflammatory cells consisted of 5×10⁴ NIH3T3: hTNFα and 5×10⁴ NIH3T3: hIL-1β cells while control cells consisted of 10×10⁴ NIH3T3 cells. Caliper measurements of the treated knee and the control knee were taken on day −1, and days 1, 2, 3 post injection. Change in joint swelling was expressed as the mean difference between the right treated knee and the left control knee as measured by caliper (FIG. 18A) and the mean AUC values over 3 days (FIG. 18B). The change in body weights by day 3 post treatment also were shown for the animals (FIG. 18C). Results represent mean±standard error of the mean, n=3 mice/group. Significance is indicated as ** with p value <0.005.

### DETAILED DESCRIPTION OF THE DISCLOSURE

### Definitions

All publications, including but not limited to disclosures and disclosure applications, cited in this specification are herein incorporated by reference as though fully set forth.

It is to be understood that the terminology used herein is for the purpose of describing particular embodiments only and is not intended to be limiting. Unless defined otherwise, all technical and scientific terms used herein have the same meaning as commonly understood by one of ordinary skill in the art to which the disclosure pertains.

Although any methods and materials similar or equivalent to those described herein may be used in the practice for testing of the present disclosure, exemplary materials and methods are described herein. In describing and claiming the present disclosure, the following terminology will be used.

As used in this specification and the appended claims, the singular forms “a,” “an,” and “the” include plural referents unless the content clearly dictates otherwise. Thus, for example, reference to “a cell” includes a combination of two or more cells, and the like.

“Antibodies” is meant in a broad sense and includes immunoglobulin molecules including monoclonal antibodies including murine, human, humanized and chimeric monoclonal antibodies, antibody fragments, bispecific or multi-specific antibodies, dimeric, tetrameric or multimeric antibodies, single chain antibodies, domain antibodies and any other modified configuration of the immunoglobulin molecule that comprises an antigen binding site of the required specificity.

“Full length antibody molecules” are comprised of two heavy chains (HC) and two light chains (LC) inter-connected by disulfide bonds as well as multimers thereof (e.g. IgM). Each heavy chain is comprised of a heavy chain variable region (V_{H}) and a heavy chain constant region (comprised of domains C_{H1}, hinge, C_{H2} and C_{H3}). Each light chain is comprised of a light chain variable region (V_{L}) and a light chain constant region (C_{L}). The V_{H} and the V_{L} regions may be further subdivided into regions of hyper variability, termed complementarity determining regions (CDR), interspersed with framework regions (FR). Each V_{H} and V_{L} is composed of three CDRs and four FR segments, arranged from amino-to-carboxyl-terminus in the following order: FR1, CDR1, FR2, CDR2, FR3, CDR3 and FR4.

“Complementarity determining regions (CDR)” are “antigen binding sites” in an antibody. CDRs may be defined using various terms: (i) Complementarity Determining Regions (CDRs), three in the V_{H} (HCDR1, HCDR2, HCDR3) and three in the V_{L} (LCDR1, LCDR2, LCDR3) are based on sequence variability (Wu et al. (1970) J Exp Med 132: 211-50 (Kabat et al., Sequences of Proteins of Immunological Interest, 5th Ed. Public Health Service, National Institutes of Health, Bethesda, Md., 1991). (ii) “Hypervariable regions,” “HVR,” or “HV,” three in the V_{H} (H1, H2, H3) and three in the V_{L} (L1, L2, L3) refer to the regions of an antibody variable domains which are hypervariable in structure as defined by Chothia and Lesk (Chothia et al. (1987) J Mol Biol 196: 901-17. The International ImMunoGeneTics (IMGT) database (http://www_imgt_org) provides a standardized numbering and definition of antigen-binding sites. The correspondence between CDRs, HVs and IMGT delineations is described in (Lefranc et al. (2003) Dev Comp Immunol 27: 55-77. The term “CDR,” “HCDR1,” “HCDR2,” “HCDR3,” “LCDR1,” “LCDR2” and “LCDR3” as used herein includes CDRs defined by any of the methods described supra, Kabat, Chothia or IMGT, unless otherwise explicitly stated in the specification.

Immunoglobulins may be assigned to five major classes, IgA, IgD, IgE, IgG and IgM, depending on the heavy chain constant region amino acid sequence. IgA and IgG are further sub-classified as the isotypes IgA₁, IgA₂, IgG₁, IgG₂, IgG₃ and IgG₄. Antibody light chains of any vertebrate species may be assigned to one of two clearly distinct types, namely kappa (κ) and lambda (λ), based on the amino acid sequences of their constant regions.

“Antibody fragments” refers to a portion of an immunoglobulin molecule that retains the heavy chain and/or the light chain antigen binding site, such as heavy chain complementarity determining regions (HCDR) 1, 2 and 3, light chain complementarity determining regions (LCDR) 1, 2 and 3, a heavy chain variable region (V_{H}), or a light chain variable region (V_{L}). Antibody fragments include well known F_{ab}, F(_{ab′})₂, F_{d} and Fᵥ fragments as well as domain antibodies (dAb) consisting of one V_{H} domain. V_{H} and V_{L} domains may be linked together via a synthetic linker to form various types of single chain antibody designs where the V_{H}/V_{L} domains may pair intramolecularly, or intermolecularly in those cases when the V_{H} and V_{L} domains are expressed by separate single chain antibody constructs, to form a monovalent antigen binding site, such as single chain Fv (scFv) or diabody; described for example in Int. Disclosure Publ. Nos. WO1998/44001, WO1988/01649, WO1994/13804 and WO1992/01047.

“Monoclonal antibody” refers to an antibody population with single amino acid composition in each heavy and each light chain, except for possible well-known alterations such as removal of C-terminal lysine from the antibody heavy chain. Monoclonal antibodies typically bind one antigenic epitope, except that bispecific monoclonal antibodies bind two distinct antigenic epitopes. Monoclonal antibodies may have heterogeneous glycosylation within the antibody population. Monoclonal antibody may be monospecific or multi-specific, or monovalent, bivalent or multivalent. A bispecific antibody is included in the term monoclonal antibody.

“Isolated antibody” refers to an antibody or antibody fragment that is substantially free of other antibodies having different antigenic specificities. “Isolated antibody” encompasses antibodies that are isolated to a higher purity, such as antibodies that are 80%, 81%, 82%, 83%, 84%, 85%, 86%, 87%, 88%, 89%, 90%, 91%, 92%, 93%, 94%, 95%, 96%, 97%, 98%, 99% or 100% pure.

“Humanized antibody” refers to an antibody in which the antigen binding sites are derived from non-human species and the variable region frameworks are derived from human immunoglobulin sequences. Humanized antibody may include substitutions in the framework so that the framework may not be an exact copy of expressed human immunoglobulin or human immunoglobulin germline gene sequences.

“Human antibody” refers to an antibody having heavy and light chain variable regions in which both the framework and the antigen binding site are derived from sequences of human origin and is optimized to have minimal immune response when administered to a human subject. If the antibody contains a constant region or a portion of the constant region, the constant region also is derived from sequences of human origin.

The numbering of amino acid residues in the antibody constant region throughout the specification is according to the EU index as described in Kabat et al., Sequences of Proteins of Immunological Interest, 5th Ed. Public Health Service, National Institutes of Health, Bethesda, Md. (1991), unless otherwise explicitly stated.

Conventional one and three-letter amino acid codes are used herein as shown in Table 1.

TABLE 1

| Amino acid | Three-letter code | One-letter code |
|---|---|---|
| Alanine | Ala | A |
| Arginine | Arg | R |
| Asparagine | Asn | N |
| Aspartate | Asp | D |
| Cysteine | Cys | C |
| Glutamate | Gln | E |
| Glutamine | Glu | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The polypeptides, nucleic acids, fusion proteins, and other compositions provided herein may encompass polypeptides, nucleic acids, fusion proteins, and the like that have a recited percent identity to an amino acid sequence or DNA sequence provided herein. The term “identity” refers to a relationship between the sequences of two or more polypeptide molecules or two or more nucleic acid molecules, as determined by aligning and comparing the sequences. “Percent identity,” “percent homology,” “sequence identity,” or “sequence homology” and the like mean the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073. In calculating percent identity, the sequences being compared are typically aligned in a way that gives the largest match between the sequences.

The constant region sequences of the mammalian IgG heavy chain are designated in sequence as C_{H1}-hinge-C_{H2}-C_{H3}. The “hinge,” “hinge region” or “hinge domain” of an IgG is generally defined as including Glu216 and terminating at Pro230 of human IgG1 according to the EU Index but functionally, the flexible portion of the chain may be considered to include additional residues termed the upper and lower hinge regions, such as from Glu216 to Gly237 and the lower hinge has been referred to as residues 233 to 239 of the F_{c} region where F_{c}γR binding was generally attributed. Hinge regions of other IgG isotypes may be aligned with the IgG₁ sequence by placing the first and last cysteine residues forming inter-heavy chain S—S bonds. Although boundaries may vary slightly, as numbered according to the EU Index, the C_{H1} domain is adjacent to the V_{H} domain and amino terminal to the hinge region of an immunoglobulin heavy chain molecule and includes the first (most amino terminal) constant region of an immunoglobulin heavy chain, e.g., from about EU positions 118-215. The F_{c} domain extends from amino acid 231 to amino acid 447; the C_{H2} domain is from about Ala231 to Lys340 or Gly341 and the C_{H3} from about Gly341 or Gln342 to Lys447. The residues of the IgG heavy chain constant region of the C_{H1} region terminate at Lys. The F_{c} domain containing molecule comprises at least the C_{H2} and the C_{H3} domains of an antibody constant region, and therefore comprises at least a region from about Ala231 to Lys447 of IgG heavy chain constant region. The F_{c} domain containing molecule may optionally comprise at least portion of the hinge region.

“Epitope” refers to a portion of an antigen to which an antibody specifically binds. Epitopes typically consist of chemically active (such as polar, non-polar or hydrophobic) surface groupings of moieties such as amino acids or polysaccharide side chains and may have specific three-dimensional structural characteristics, as well as specific charge characteristics. An epitope may be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule. Antibody “epitope” depends on the methodology used to identify the epitope.

A “leader sequence” as used herein includes any signal peptide that can be processed by a mammalian cell, including the human B2M leader. Such sequences are well-known in the art.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. The terms also include polypeptides that have co-translational (e.g., signal peptide cleavage) and post-translational modifications of the polypeptide, such as, for example, disulfide-bond formation, glycosylation, acetylation, phosphorylation, proteolytic cleavage, and the like.

Furthermore, as used herein, a “polypeptide” refers to a protein that includes modifications, such as deletions, additions, and substitutions (generally conservative in nature as would be known to a person in the art) to the native sequence, as long as the protein maintains the desired activity. These modifications can be deliberate, as through site-directed mutagenesis, or can be accidental, such as through mutations of hosts that produce the proteins, or errors due to PCR amplification or other recombinant DNA methods.

The term “recombinant,” as used herein to describe a nucleic acid molecule, means a polynucleotide of genomic, cDNA, viral, semisynthetic, and/or synthetic origin, which, by virtue of its origin or manipulation, is not associated with all or a portion of the polynucleotide sequences with which it is associated in nature. The term “recombinant,” as used with respect to a protein or polypeptide, refers to a polypeptide produced by expression from a recombinant polynucleotide. The term “recombinant,” as used with respect to a host cell or a virus, refers to a host cell or virus into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

The terms “polynucleotide,” “oligonucleotide,” “nucleic acid” and “nucleic acid molecule” are used interchangeably herein to include a polymeric form of nucleotides, either ribonucleotides or deoxyribonucleotides. This term refers only to the primary structure of the molecule.

“Vector” refers to a polynucleotide capable of being duplicated within a biological system or that can be moved between such systems. Vector polynucleotides typically contain elements, such as origins of replication, polyadenylation signal or selection markers, that function to facilitate the duplication or maintenance of these polynucleotides in a biological system, such as a cell, virus, animal, plant, and reconstituted biological systems utilizing biological components capable of duplicating a vector. The vector polynucleotide may be DNA or RNA molecules, cDNA, or a hybrid of these, single stranded or double stranded.

“Expression vector” refers to a vector that can be utilized in a biological system or in a reconstituted biological system to direct the translation of a polypeptide encoded by a polynucleotide sequence present in the expression vector.

“Valent” refers to the presence of a specified number of binding sites specific for an antigen in a molecule. As such, the terms “monovalent,” “bivalent,” “tetravalent,” and “hexavalent” refer to the presence of one, two, four and six binding sites, respectively, specific for an antigen in a molecule.

As used herein, the term “heterologous” used in reference to nucleic acid sequences, proteins or polypeptides, means that these molecules are not naturally occurring in the cell from which the heterologous nucleic acid sequence, protein or polypeptide was derived. For example, the nucleic acid sequence coding for a human polypeptide that is inserted into a cell that is not a human cell is a heterologous nucleic acid sequence in that particular context. Whereas heterologous nucleic acids may be derived from different organism or animal species, such nucleic acid need not be derived from separate organism species to be heterologous. For example, in some instances, a synthetic nucleic acid sequence or a polypeptide encoded therefrom may be heterologous to a cell into which it is introduced in that the cell did not previously contain the synthetic nucleic acid. As such, a synthetic nucleic acid sequence or a polypeptide encoded therefrom may be considered heterologous to a human cell, e.g., even if one or more components of the synthetic nucleic acid sequence or a polypeptide encoded therefrom was originally derived from a human cell.

A “host cell,” as used herein, denotes an in vivo or in vitro eukaryotic cell or a cell from a multicellular organism (e.g., a cell line) cultured as a unicellular entity, which eukaryotic cells can be, or have been, used as recipients for a nucleic acid (e.g., an expression vector that comprises a nucleotide sequence encoding a multimeric polypeptide of the present disclosure), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a genetically modified eukaryotic host cell is genetically modified by virtue of introduction into a suitable eukaryotic host cell a heterologous nucleic acid, e.g., an exogenous nucleic acid that is foreign to the eukaryotic host cell, or a recombinant nucleic acid that is not normally found in the eukaryotic host cell.

“Specific binding” or “specifically binds” or “binds” refer to an antibody binding to a specific antigen with greater affinity than for other antigens. Typically, the antibody “specifically binds” when the equilibrium dissociation constant (K_{D}) for binding is about 1×10⁻⁸ M or less, for example about 1×10⁻⁹ M or less, about 1×10⁻¹⁰ M or less, about 1×10⁻¹¹M or less, or about 1×10⁻¹² M or less, typically with the K_{D} that is at least one hundred-fold less than its K_{D} for binding to a non-specific antigen (e.g., BSA, casein). The K_{D} may be measured using standard procedures.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (e.g., rats, mice), lagomorphs (e.g., rabbits), non-human primates, humans, canines, felines, ungulates (e.g., equines, bovines, ovines, porcines, caprines), etc.

A “therapeutically effective amount” or “efficacious amount” refers to the amount of an agent, or combined amounts of two agents, that, when administered to a mammal or other subject for treating a disease, is sufficient to affect such treatment for the disease. The “therapeutically effective amount” will vary depending on the agent(s), the disease and its severity and the age, weight, etc., of the subject to be treated.

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

### Anti-TNFα Antibody

Tumor necrosis factor alpha (TNFα), originally discovered due to its antitumor cell properties, has since been shown to mediate the inflammatory response and modulate immune function (Aggarwal 2003). TNFα is produced by macrophages, immune cells and granulocytes and expressed as a membrane protein on the cell surface that is rapidly released via proteolytic cleavage by ADAM-17. The active form of soluble TNFα is a homotrimer which signals via two receptors, TNFRI and TNFRII. While the normal functions of TNFα are beneficial, uncontrolled excessive production of TNFα can lead to chronic disease (Feldmann, Brennan et al. 2004).

Infliximab (Remicade®, cA2) is a chimeric antibody comprised of human light and heavy chain constant domains and murine light and heavy variable domains developed by Centocor/Janssen. Infliximab has been shown to bind TNFα with high specificity and affinity, thereby neutralizing the biologic functions of TNFα. Infliximab has completed clinical trials and received regulatory approval for Crohn's disease (1998), rheumatoid arthritis (1999), ankylosing spondylitis (2004), psoriatic arthritis (2005), ulcerative colitis (2005), plaque psoriasis (2006). In particular, the mechanism of action for infliximab in rheumatoid arthritis has been well-documented (Monaco, Nanchahal et al. 2015).

Adalimumab (Humira®, D2E7), developed by Abbott/Abbvie, is an engineered human monoclonal antibody comprised of human heavy and light chains with variable domains optimized by phage display technology. The mechanism of action for adalimumab is quite similar to infliximab (Kaymakcalan, Sakorafas et al. 2009). Beginning in 2002, adalimumab has been approved for the same indications as infliximab, with the addition of polyarticular juvenile idiopathic arthritis, hidradenitis suppurativa and uveitis.

Certolizumab pegol (Cimzia®, CDP-870) is an antibody fragment, developed by UCB, that targets TNFα. It is a humanized Fab fragment comprised of murine heavy and light variable sequences interspliced with human variable framework sequences attached to human heavy CH1 and light chain constant domains, respectively. A polyethylene glycol moiety is attached to extend the serum half-life of the molecule. Certolizumab pegol binds and neutralizes the effect of TNFα much like infliximab and adalimumab, however it lacks an Fc domain and hence Fc-dependent extended half-life and potential cell lysis. Beginning in 2008, certolizumab pegol has received regulatory approval for Crohn's disease, rheumatoid arthritis, psoriatic arthritis, ankylosing spondylitis and plaque psoriasis.

A fourth anti-TNFα, golimumab (Simponi) was developed by Janssen Biotech. It is a fully human antibody generated in human antibody transgenic mice (Shealy, Cai et al. 2010). Golimumab has a mechanism of action similar to infliximab, adalimumab and certolizumab pegol. Golimumab received initial regulatory approval for rheumatoid arthritis, psoriatic arthritis and ankylosing spondylitis in 2009, with a further approval for ulcerative colitis in 2013.

As part of the bispecific antibodies and antigen-binding fragments thereof with dual specificity that specifically bind and neutralize, inhibit, block, abrogate, reduce, or interfere with both tumor necrosis factor alpha (TNFα) and interleukin 1β (IL-1β), herein is described human monoclonal antibodies and antigen binding fragments that specifically bind tumor necrosis factor α (TNF-α) and neutralize the functional activity of TNF-α to its receptor. The activity of TNFα that can be neutralized, inhibited, blocked, abrogated, reduced or interfered with, by the antibodies or fragments thereof of the disclosure, includes, but not by the way of limitation, neutralization of TNFα activation of its receptor, and the like. In one embodiment, an antibody or fragment thereof of the present disclosure can neutralize, inhibit, block, abrogate, reduce or interfere with, an activity of TNFα by binding to an epitope of TNFα that is directly involved in the targeted activity of TNFα. In another embodiment, an antibody or fragment thereof of the disclosure can neutralize, inhibit, block, abrogate, reduce or interfere with, an activity of TNFα by binding to an epitope of TNFα that is not directly involved in the targeted activity of TNFα, but the antibody or fragment binding thereto sterically or conformationally inhibits, blocks, abrogates, reduces or interferes with, the targeted activity of TNFα. In yet another embodiment, an antibody or fragment thereof of the disclosure binds to an epitope of TNFα that is not directly involved in the targeted activity of TNFα (i.e., a non-blocking antibody), but the antibody or fragment binding thereto results in the enhancement of the clearance of TNFα.

As a non-limiting example, the disclosure provides for nine anti-TNFα antibody heavy chain variable domain sequences, designated as ADA-H, ADA-H1, ADA-H1X, ADA-H2, ADA-H2X, ADA-H3, ADA-H3X, ADAH4, ADAH4X, with amino acid sequences set forth as SEQ ID NO. 1, NO. 2, NO. 3, NO 4, NO 5, NO 6, NO 7, NO 8, NO 9, respectively. In embodiments, the disclosure provides an anti-TNFα antibody comprising a heavy chain variable domain comprising an amino acid sequence with at least about 80%, about 85%, about 90%, about 95%, or about 99% sequence identity to SEQ ID NO: 1, 2, 3 4, 5, 6, 7, 8, or 9.

As a non-limiting example, the disclosure provides for three anti-TNFα antibody light chain variable domain sequences, designated as ADA-L, ADA-L1, ADA-L2, with amino acid sequences set forth as SEQ ID NO. 10, NO. 11, NO. 12, respectively. In embodiments, the disclosure provides an anti-TNFα antibody comprising a light chain variable domain comprising an amino acid sequence with at least about 80%, about 85%, about 90%, about 95%, or about 99% sequence identity to SEQ ID NO: 10, 11, or 12.

As a non-limiting example, the disclosure provides for an anti-TNFα antibody heavy chain sequence based on heavy chain variable domain ADA-H with IgG1 F_{c} with F405L mutation, designated as EAC33, with amino acid sequences set forth as SEQ ID NO. 13. The disclosure also provides for nine anti-TNFα antibody heavy chain sequences based on heavy chain variable domains ADA-H, ADA-H1, ADA-H1X, ADA-H2, ADA-H2X, ADA-H3, ADA-H3X, ADA-H4, ADA-H4X with IgG1 Fc with L234A, L235A, F405L, M428L, N434S mutations, designated as EAC119, EAC129, EAC130, EAC131, EAC132, EAC133, EAC134, EAC135, EAC136, respectively, with amino acid sequences set forth as SEQ ID NO. 14, NO. 15, NO. 16, NO 17, NO 18, NO 19, NO 20, NO 21, NO 22, respectively. The disclosure also provides for five anti-TNFα antibody heavy chain sequences based on heavy chain variable domains ADA-H, ADA-H1X, ADA-H2X, ADA-H3X, ADA-H4X with IgG1 F_{c} with E233P, L234A, L235A, F405L, M428L, N434S mutations and G236 deleted, designated as EAC144, EAC166, EAC167, EAC168, EAC169, respectively, with amino acid sequences set forth as SEQ ID NO. 23, NO. 24, NO. 25, NO 26, NO 27, respectively. In embodiments, the disclosure provides an anti-TNFα antibody comprising a heavy chain amino acid sequence having at least about 80%, at least about 85%, at least about 90%, at least about 95%, or at least about 99% sequence identity to SEQ ID NO: 13, 14, 15, 16, 17, 18, 19, 20, 21, 22, 23, 24, 25, 26, or 27.

As a non-limiting example, the disclosure provides for an anti-TNFα antibody light chain sequences based on light chain variable domains ADA-L, ADA-L1, ADA-L2, designated as EAC34, EAC127, EAC128, respectively, with amino acid sequences set forth as SEQ ID NO. 28, NO 29, NO 30, respectively. In embodiments, the disclosure provides an anti-TNFα antibody comprising a light chain amino acid sequence having at least about 80%, at least about 85%, at least about 90%, at least about 95%, or at least about 99% sequence identity to SEQ ID NO: 28, 29, or 30.

As a non-limiting example, by pairing anti-TNFα antibody heavy chain sequences and anti-TNFα antibody light chain sequences described above, the disclosure provides anti-TNFα antibodies listed in Table 2 with combinations of different heavy chain variable domains and different light chain variable domains with different IgG Fc.

TABLE 2
anti-TNFα antibody

| Anti-TNFα | Heavy | Light | | | |
|---|---|---|---|---|---|
| Antibody | chain | chain | V_{H} | V_{L} | Fc |
| TAVO3334 | EAC33 | EAC34 | ADA-H | ADA-L | IgG1 with F405L mutation |
| TAVO11934 | EAC119 | EAC34 | ADA-H | ADA-L | IgG1 with L234A, L235A, F405L, |
| | | | | | M428L, N434S mutations |
| TAVO129127 | EAC129 | EAC127 | ADA-H1 | ADA-L1 | IgG1 with L234A, L235A, F405L, |
| | | | | | M428L, N434S mutations |
| TAVO130127 | EAC130 | EAC127 | ADA-H1X | ADA-L1 | IgG1 with L234A, L235A, F405L, |
| | | | | | M428L, N434S mutations |
| TAVO131127 | EAC131 | EAC127 | ADA-H2 | ADA-L1 | IgG1 with L234A, L235A, F405L, |
| | | | | | M428L, N434S mutations |
| TAVO132127 | EAC132 | EAC127 | ADA-H2X | ADA-L1 | IgG1 with L234A, L235A, F405L, |
| | | | | | M428L, N434S mutations |
| TAVO133127 | EAC133 | EAC127 | ADA-H3 | ADA-L1 | IgG1 with L234A, L235A, F405L, |
| | | | | | M428L, N434S mutations |
| TAVO134127 | EAC134 | EAC127 | ADA-H3X | ADA-L1 | IgG1 with L234A, L235A, F405L, |
| | | | | | M428L, N434S mutations |
| TAVO135127 | EAC135 | EAC127 | ADA-H4 | ADA-L1 | IgG1 with L234A, L235A, F405L, |
| | | | | | M428L, N434S mutations |
| TAVO136127 | EAC136 | EAC127 | ADA-H4X | ADA-L1 | IgG1 with L234A, L235A, F405L, |
| | | | | | M428L, N434S mutations |
| TAVO129128 | EAC129 | EAC128 | ADA-H1 | ADA-L2 | IgG1 with L234A, L235A, F405L, |
| | | | | | M428L, N434S mutations |
| TAVO130128 | EAC130 | EAC128 | ADA-H1X | ADA-L2 | IgG1 with L234A, L235A, F405L, |
| | | | | | M428L, N434S mutations |
| TAVO131128 | EAC131 | EAC128 | ADA-H2 | ADA-L2 | IgG1 with L234A, L235A, F405L, |
| | | | | | M428L, N434S mutations |
| TAVO132128 | EAC132 | EAC128 | ADA-H2X | ADA-L2 | IgG1 with L234A, L235A, F405L, |
| | | | | | M428L, N434S mutations |
| TAVO133128 | EAC133 | EAC128 | ADA-H3 | ADA-L2 | IgG1 with L234A, L235A, F405L, |
| | | | | | M428L, N434S mutations |
| TAVO134128 | EAC134 | EAC128 | ADA-H3X | ADA-L2 | IgG1 with L234A, L235A, F405L, |
| | | | | | M428L, N434S mutations |
| TAVO135128 | EAC135 | EAC128 | ADA-H4 | ADA-L2 | IgG1 with L234A, L235A, F405L, |
| | | | | | M428L, N434S mutations |
| TAVO136128 | EAC136 | EAC128 | ADA-H4X | ADA-L2 | IgG1 with L234A, L235A, F405L, |
| | | | | | M428L, N434S mutations |
| TAVO14434 | EAC144 | EAC34 | ADA-H | ADA-L | IgG1 with E233P, L234A, L235A, |
| | | | | | F405L, M428L, N434S mutations |
| | | | | | and G236 deleted |
| TAVO166127 | EAC166 | EAC127 | ADA-H1X | ADA-L1 | IgG1 with E233P, L234A, L235A, |
| | | | | | F405L, M428L, N434S mutations |
| | | | | | and G236 deleted |
| TAVO167127 | EAC167 | EAC127 | ADA-H2X | ADA-L1 | IgG1 with E233P, L234A, L235A, |
| | | | | | F405L, M428L, N434S mutations |
| | | | | | and G236 deleted |
| TAVO168127 | EAC168 | EAC127 | ADA-H3X | ADA-L1 | IgG1 with E233P, L234A, L235A, |
| | | | | | F405L, M428L, N434S mutations |
| | | | | | and G236 deleted |
| TAVO169127 | EAC169 | EAC127 | ADA-H4X | ADA-L1 | IgG1 with E233P, L234A, L235A, |
| | | | | | F405L, M428L, N434S mutations |
| | | | | | and G236 deleted |
| TAVO166128 | EAC166 | EAC128 | ADA-H1X | ADA-L2 | IgG1 with E233P, L234A, L235A, |
| | | | | | F405L, M428L, N434S mutations |
| | | | | | and G236 deleted |
| TAVO167128 | EAC167 | EAC128 | ADA-H2X | ADA-L2 | IgG1 with E233P, L234A, L235A, |
| | | | | | F405L, M428L, N434S mutations |
| | | | | | and G236 deleted |
| TAVO168128 | EAC168 | EAC128 | ADA-H3X | ADA-L2 | IgG1 with E233P, L234A, L235A, |
| | | | | | F405L, M428L, N434S mutations |
| | | | | | and G236 deleted |
| TAVO169128 | EAC169 | EAC128 | ADA-H4X | ADA-L2 | IgG1 with E233P, L234A, L235A, |
| | | | | | F405L, M428L, N434S mutations |
| | | | | | and G236 deleted |

### Anti-IL-1β Antibody

IL-1β is a pro-inflammatory cytokine that acts as mediator of the peripheral immune response during infection and inflammation. IL-1β is initially synthesized in the form of a precursor peptide (pro-IL-13) that is cleaved in the inflammasome complex by caspase-1, and secreted into the extracellular space. IL-1β can be released by various cell types.

There are two IL-1 receptors, IL-1RI and IL-1RII. IL-1β exerts its action on target cells through the receptor IL-1RI. Dysregulated IL-1β activity is characteristic of autoimmune diseases and may occur due to either abnormally increased levels of the cytokine, or qualitative or quantitative deficiency of IL-1RI endogenous antagonist. IL-1β is specifically implicated in several auto-inflammatory diseases.

Canakinumab (Ilaris, ACZ885) is a human monoclonal antibody targeted at interleukin-10 developed by Novartis. Its mode of action is based on the neutralization of IL-1β signalling. Canakinumab was approved for the treatment of cryopyrin-associated periodic syndromes (CAPS) in 2009, and was subsequently approved in 2016 on three additional rare and serious auto-inflammatory diseases (Gram 2016). Gevokizumab (XOMA052) is another monoclonal antibody targeting IL-1β developed by XOMA. Gevokizumab is claimed to be a regulatory therapeutic antibody that modulates IL-1β bioactivity by reducing the affinity for its IL-1RIIL-1RAcP signalling complex (Issafras, Corbin et al. 2013).

In recent years, IL-1β has been found to be associated with several steps in the development of atherosclerotic plaques, as well as other cardiovascular disease modifiers (McCarty and Frishman 2014). The hypothesis is that these inflammatory chemicals may prevent the heart from healing from damage from previous heart attacks. In 2017, a phase III clinical trial with Canakinumab revealed a 15% reduction in deaths from heart attacks, stroke and cardiovascular disease combined. Besides, the trial also revealed a significant reduction in lung cancer incidence and mortality.

As part of the bispecific antibodies and antigen-binding fragments thereof with dual specificity that specifically bind and neutralize, inhibit, block, abrogate, reduce, or interfere with both tumor necrosis factor alpha (TNFα) and interleukin 1β (IL-1β), herein is described a novel human monoclonal antibody and antigen binding fragment that specifically binds human interleukin 1β (IL-1β) and neutralizes the functional activity of IL-1β to its receptor IL-1RI. In one embodiment, an antibody or fragment thereof of the present disclosure can neutralize, inhibit, block, abrogate, reduce or interfere with, an activity of IL-1β by binding to an epitope of IL-1β that is directly involved in the targeted activity of IL-1β. In another embodiment, an antibody or fragment thereof of the disclosure can neutralize, inhibit, block, abrogate, reduce or interfere with, an activity of IL-1β by binding to an epitope of IL-1β that is not directly involved in the targeted activity of IL-1β, but the antibody or fragment binding thereto sterically or conformationally inhibits, blocks, abrogates, reduces or interferes with, the targeted activity of IL-1β. In yet another embodiment, an antibody or fragment thereof of the disclosure binds to an epitope of IL-1β that is not directly involved in the targeted activity of IL-1β (i.e., a non-blocking antibody), but the antibody or fragment binding thereto results in the enhancement of the clearance of IL-1β.

As a non-limiting example, the disclosure provides for three anti-IL-1β antibody heavy chain variable domain sequences, designated as Ab5H3, Ab8H1, Ab9H1, with amino acid sequences set forth as SEQ ID NO. 31, NO. 32, NO. 33, respectively. In embodiments, the disclosure provides for an anti-IL-1β antibody comprising a heavy chain variable domain comprising an amino acid sequence with at least about 80%, about 85%, about 90%, about 95%, or about 99% sequence identity to SEQ ID NO: 31, 32, or 33.

As a non-limiting example, the disclosure provides for three anti-IL-1β antibody light chain variable domain sequences, designated as Ab5L, Ab8L3, Ab9L1, with amino acid sequences set forth as SEQ ID NO. 34, NO. 35, NO. 36, respectively. In embodiments, the disclosure provides for an anti-IL-1β antibody comprising a light chain variable domain comprising an amino acid sequence with at least about 80%, about 85%, about 90%, about 95%, or about 99% sequence identity to SEQ ID NO: 34, 35, or 36.

As a non-limiting example, the disclosure provides for three anti-IL-1β antibody heavy chain sequences based on heavy chain variable domains Ab5H3, Ab8H1, Ab9H1, with IgG1 F_{c} with K409R mutation, designated as EAC53, EAC73, EAC80, with amino acid sequences set forth as SEQ ID NO. 37, NO. 38, NO. 39, respectively. The disclosure also provides for two anti-IL-1β antibody heavy chain sequences based on heavy chain variable domains Ab5H3 and Ab8H1 with IgG1 Fc with L234A, L235A, K409R, M428L, N434S mutations, designated as EAC120 and EAC121, with amino acid sequences set forth as SEQ ID NO. 40 and NO. 41, respectively. The disclosure also provides for two anti-IL-1β antibody heavy chain sequences based on heavy chain variable domains Ab8H1 and Ab9H1 with IgG1 Fc with E233P, L234A, L235A, K409R, M428L, N434S mutations and G236 deleted, designated as EAC145 and EAC161, with amino acid sequences set forth as SEQ ID NO. 42 and NO. 43, respectively. In embodiments, the disclosure provides an anti-IL-1β antibody comprising a heavy chain amino acid sequence having at least about 80%, at least about 85%, at least about 90%, at least about 95%, or at least about 99% sequence identity to SEQ ID NO: 37, 38, 39, 40, 41, 42, or 43.

As a non-limiting example, the disclosure provides for three anti-IL-1β antibody light chain sequences based on light chain variable domains Ab5L, Ab8L3, Ab9L1, designated as EAC32, EAC78, EAC83, with amino acid sequences set forth as SEQ ID NO. 44, NO. 45, NO. 46, respectively. In embodiments, the disclosure provides an anti-IL-1β antibody comprising a light chain amino acid sequence having at least about 80%, at least about 85%, at least about 90%, at least about 95%, or at least about 99% sequence identity to SEQ ID NO: 44, 45, or 46.

As a non-limiting example, by pairing anti-IL-1β antibody heavy chain sequences and anti-IL-1β antibody light chain sequences described above, the disclosure provides exemplary anti-IL-1β antibodies listed in Table 3 with combinations of different heavy chain variable domains and different light chain variable domains with different IgG Fc.

TABLE 3
anti-IL1β antibody

| Anti-IL1β | Heavy | Light | | | |
|---|---|---|---|---|---|
| Antibody | chain | chain | V_{H} | V_{L} | Fc |
| TAVO5332 | EAC53 | EAC32 | Ab5-H3 | Ab5-L | IgG1 with K409R mutation |
| TAVO7378 | EAC73 | EAC78 | Ab8-H1 | Ab8-L3 | IgG1 with K409R mutation |
| TAVO8083 | EAC80 | EAC83 | Ab9-H1 | Ab9-L1 | IgG1 with K409R mutation |
| TAVO12032 | EAC120 | EAC32 | Ab5-H3 | Ab5-L | IgG1 with L234A, L235A, K409R, |
| | | | | | M428L, N434S mutations |
| TAVO12178 | EAC121 | EAC78 | Ab8-H1 | Ab8-L3 | IgG1 with L234A, L235A, K409R, |
| | | | | | M428L, N434S mutations |
| TAVO14578 | EAC145 | EAC78 | Ab8-H1 | Ab8-L3 | IgG1 with E233P, L234A, L235A, |
| | | | | | K409R, M428L, N434S mutations |
| | | | | | and G236 deleted |
| TAVO16183 | EAC161 | EAC83 | Ab9-H1 | Ab9-L1 | IgG1 with E233P, L234A, L235A, |
| | | | | | F405L, M428L, N434S mutations |
| | | | | | and G236 deleted |

The disclosure also provides for mixtures of the anti-IL1β and anti-TNFα antibodies provided herein. For example, the disclosure provides compositions comprising any one or more of the anti-IL1β antibodies provided herein with any one or more of the anti-TNFα antibodies provided herein. For example, in embodiments, the present disclosure provides a composition comprising an anti-IL1β antibody or fragment thereof comprising a heavy chain variable domain comprising an amino acid sequence with at least about 80%, about 85%, about 90%, about 95%, or about 99% sequence identity, or 100% sequence identity, to SEQ ID NO: 31, 32, or 33 and a light chain variable domain comprising an amino acid sequence with at least about 80%, about 85%, about 90%, about 95%, or about 99% sequence identity, or 100% sequence identity, to SEQ ID NO: 34, 35, or 36; and an antibody or fragment thereof that is specific for TNFα. In embodiments, the disclosure also provides methods of use of such mixtures of antibodies.

### Anti-TNFα and IL-1β Bispecific Antibody

Bispecific antibodies are new development in the pharmaceutical industry and they can recognize two different targets, often additive or synergistic in nature (Labrijn, Janmaat et al. 2019). Such dual specificity allows inhibition of two different signaling pathways at the same time as well as dual targeting of different pathogenic mediators. Such approach would likely improve treatment options against autoimmune diseases as well as other inflammatory conditions.

Bispecific antibodies or fragments can be of several configurations. For example, bispecific antibodies may resemble single antibodies (or antibody fragments) but have two different antigen binding sites (variable regions) and may be bivalent or monovalent. Various bispecific antibody formats are known to the ordinarily skilled person. Bispecific antibody formats include, for example, full IgG-like bispecific antibodies (such as those generated using controlled Fab-arm exchange technique described herein), knob-in-hole antibodies, DuoBody® antibodies, scFv₂-Fc bispecific antibodies which have an Fc region and two scFv portions (e.g., ADAPTIR™), bispecific T-cell engager (BiTE)-based antibodies such as BiTE/ScFv₂, dual-affinity re-targeting antibody (DART)-based bispecific antibodies including DART binding regions with or without an Fc portion, DNL-Fab₃ bispecific antibodies, scFv-HAS-scFv bispecific antibodies, and DVD-Ig bispecific antibodies.

Both TNFα and IL-1β are pro-inflammatory cytokines that act as mediators of the peripheral immune response during infection and inflammation. However, excess production of both TNFα and IL-1β correlates with the initiation and progression of many types of medical problems including: autoimmune/inflammatory diseases; diabetes, nerve, eye, skin disease conditions; various types of cancers; endocrinology dysfunction; and disruption of normal wound healing. Therefore, neutralizing the activities of both TNFα and IL-1β may provide a therapeutic for these inflammatory diseases or any other disorders caused by excess TNFα and IL-1β. The current disclosure brings together a newly re-engineered, dual-specific, anti-TNFα and IL-1β antibody which could offer dual TNFα and IL-1β cytokines neutralization in specific cell types. Moreover, additional antibody engineering applied to the novel bispecific antibody also offers altered in vivo half-life, better safety profile as well as effector function via differing affinities for FcR. This provides not only synergy in efficacy but also better dose-titration for patients with different inflammatory conditions who would likely have different needs.

Accordingly, the present disclosure provides bispecific antibodies and antigen-binding fragments thereof with dual specificity that specifically bind and neutralize, inhibit, block, abrogate, reduce, or interfere with both tumor necrosis factor alpha (TNFα) and interleukin 1 β (IL-1β). The activity of TNFα and IL-1β that can be neutralized, inhibited, blocked, abrogated, reduced or interfered with, by the bispecific antibodies or fragments thereof of the disclosure, includes, but not by the way of limitation, neutralization of TNFα and IL-1β activation of their receptors, and the like.

As a non-limiting example, the disclosure provides for bispecific antibodies and antigen-binding fragments constituted with nine anti-TNFα antibody heavy chain variable domain sequences, designated as ADA-H, ADA-H1, ADA-H1X, ADA-H2, ADA-H2X, ADA-H3, ADA-H3X, ADAH4, ADAH4X, with amino acid sequences set forth as SEQ ID NO. 1, NO. 2, NO. 3, NO 4, NO 5, NO 6, NO 7, NO 8, NO 9, respectively. In embodiments, the bispecific antibodies and antigen-binding fragments comprise an anti-TNFα antibody heavy chain variable domain comprising an amino acid sequence having at least about 80%, at least 85%, at least 90%, at least 95%, or at least 99% sequence identity to SEQ ID NO: 1, 2, 3, 4, 5, 6, 7, 8, or 9.

As a non-limiting example, the disclosure provides for bispecific antibodies and antigen-binding fragments constituted with three anti-TNFα antibody light chain variable domain sequences, designated as ADA-L, ADA-L1, ADA-L2, with amino acid sequences set forth as SEQ ID NO. 10, NO. 11, NO. 12, respectively. In embodiments, the bispecific antibodies and antigen-binding fragments comprise an anti-TNFα antibody light chain variable domain comprising an amino acid sequence having at least about 80%, at least 85%, at least 90%, at least 95%, or at least 99% sequence identity to SEQ ID NO: 10, 11, or 12.

As a non-limiting example, the disclosure provides for bispecific antibodies and antigen-binding fragments constituted with an anti-TNFα antibody heavy chain sequence based on heavy chain variable domain ADA-H with IgG1 Fc with F405L mutation, designated as EAC33, with amino acid sequences set forth as SEQ ID NO. 13. The disclosure also provides for bispecific antibodies and antigen-binding fragments constituted with nine anti-TNFα antibody heavy chain sequences based on heavy chain variable domains ADA-H, ADA-H1, ADA-H1X, ADA-H2, ADA-H2X, ADA-H3, ADA-H3X, ADA-H4, ADA-H4X with IgG1 Fc with L234A, L235A, F405L, M428L, N434S mutations, designated as EAC119, EAC129, EAC130, EAC131, EAC132, EAC133, EAC134, EAC135, EAC136, respectively, with amino acid sequences set forth as SEQ ID NO. 14, NO. 15, NO. 16, NO 17, NO 18, NO 19, NO 20, NO 21, NO 22, respectively. The disclosure also provides for bispecific antibodies and antigen-binding fragments constituted with five anti-TNFα antibody heavy chain sequences based on heavy chain variable domains ADA-H, ADA-H1X, ADA-H2X, ADA-H3X, ADA-H4X with IgG1 Fc with E233P, L234A, L235A, F405L, M428L, N434S mutations and G236 deleted, designated as EAC144, EAC166, EAC167, EAC168, EAC169, respectively, with amino acid sequences set forth as SEQ ID NO. 23, NO. 24, NO. 25, NO 26, NO 27, respectively. In embodiments, the disclosure provides a bispecific antibody comprising a heavy chain amino acid sequence having at least about 80%, at least about 85%, at least about 90%, at least about 95%, or at least about 99% sequence identity to SEQ ID NO: 13, 14, 15, 16, 17, 18, 19, 20, 21, 22, 23, 24, 25, 26, or 27.

As a non-limiting example, the disclosure provides for bispecific antibodies and antigen-binding fragments constituted with an anti-TNFα antibody light chain sequences based on light chain variable domains ADA-L, ADA-L1, ADA-L2, designated as EAC34, EAC127, EAC128, respectively, with amino acid sequences set forth as SEQ ID NO. 28, NO 29, NO 30, respectively. In embodiments, the disclosure provides a bispecific antibody comprising a light chain amino acid sequence having at least about 80%, at least about 85%, at least about 90%, at least about 95%, or at least about 99% sequence identity to SEQ ID NO: 28, 29, or 30.

As a non-limiting example, by pairing anti-TNFα antibody heavy chain sequences and anti-TNFα antibody light chain sequences described above, the disclosure provides bispecific antibodies and antigen-binding fragments constituted with anti-TNFα antibodies listed in Table 2 with combinations of different heavy chain variable domains and different light chain variable domains with different IgG F_{c}.

As a non-limiting example, the disclosure provides for bispecific antibodies and antigen-binding fragments constituted with three anti-IL-1β antibody heavy chain variable domain sequences, designated as Ab5H3, Ab8H1, Ab9H1, with amino acid sequences set forth as SEQ ID NO. 31, NO. 32, NO. 33, respectively. In embodiments, the bispecific antibodies and antigen-binding fragments comprise an anti-IL-1β antibody heavy chain variable domain comprising an amino acid sequence having at least about 80%, at least 85%, at least 90%, at least 95%, or at least 99% sequence identity to SEQ ID NO: 31, 32, or 33.

As a non-limiting example, the disclosure provides for bispecific antibodies and antigen-binding fragments constituted with three anti-IL-1β antibody light chain variable domain sequences, designated as Ab5L, Ab8L3, Ab9L1, with amino acid sequences set forth as SEQ ID NO. 34, NO. 35, NO. 36, respectively. In embodiments, the bispecific antibodies and antigen-binding fragments comprise an anti-IL-1β antibody light chain variable domain comprising an amino acid sequence having at least about 80%, at least 85%, at least 90%, at least 95%, or at least 99% sequence identity to SEQ ID NO: 34, 35, or 36.

As a non-limiting example, the disclosure provides for bispecific antibodies and antigen-binding fragments constituted with three anti-IL-1β antibody heavy chain sequences based on heavy chain variable domains Ab5H3, Ab8H1, Ab9H1, with IgG1 Fc with K409R mutation, designated as EAC53, EAC73, EAC80, with amino acid sequences set forth as SEQ ID NO. 37, NO. 38, NO. 39, respectively. The disclosure also provides for bispecific antibodies and antigen-binding fragments constituted with two anti-IL-1β antibody heavy chain sequences based on heavy chain variable domains Ab5H3 and Ab8H1 with IgG1 Fc with L234A, L235A, K409R, M428L, N434S mutations, designated as EAC120 and EAC121, with amino acid sequences set forth as SEQ ID NO. 40 and NO. 41, respectively. The disclosure also provides for bispecific antibodies and antigen-binding fragments constituted with two anti-IL-1β antibody heavy chain sequences based on heavy chain variable domains Ab8H1 and Ab9H1 with IgG1 Fc with E233P, L234A, L235A, K409R, M428L, N434S mutations and G236 deleted, designated as EAC145 and EAC161, with amino acid sequences set forth as SEQ ID NO. 42 and NO. 43, respectively. In embodiments, the disclosure provides a bispecific antibody comprising a heavy chain amino acid sequence having at least about 80%, at least about 85%, at least about 90%, at least about 95%, or at least about 99% sequence identity to SEQ ID NO: 37, 38, 39, 40, 41, 42, or 43.

As a non-limiting example, the disclosure provides for bispecific antibodies and antigen-binding fragments constituted with three anti-IL-1β antibody light chain sequences based on light chain variable domains Ab5L, Ab8L3, Ab9L1, designated as EAC32, EAC78, EAC83, with amino acid sequences set forth as SEQ ID NO. 44, NO. 45, NO. 46, respectively. In embodiments, the disclosure provides a bispecific antibody comprising a light chain amino acid sequence having at least about 80%, at least about 85%, at least about 90%, at least about 95%, or at least about 99% sequence identity to SEQ ID NO: 44, 45, or 46.

As a non-limiting example, by pairing anti-IL-1β antibody heavy chain sequences and anti-IL-1β antibody light chain sequences described above, the disclosure provides bispecific antibodies and antigen-binding fragments constituted with anti-IL1β antibodies listed in Table 3 with combinations of different heavy chain variable domains and different light chain variable domains with different IgG Fc.

As a non-limiting example, the disclosure provides for bispecific antibodies with dual specificity to both TNFα and IL-1β listed in Table 4 with combination of anti-TNFα antibodies listed in Table 2 and anti-IL-1β antibodies listed in Table 3 with different IgG Fc.

TABLE 4
anti-TNFα and IL-1β bispecific antibody

| | TNFα | TNFα | IL-Iβ | IL-Iβ | |
|---|---|---|---|---|---|
| TNFα × IL-1β | Heavy chain | Light chain | Heavy chain | Light chain | |
| bispecific antibody | (VH) | (VL) | (VH) | (VL) | Fc |
| TAVO3334 × 5332 | EAC33 | EAC34 | EAC53 | EAC32 | IgG1 |
| | (ADA-H) | (ADA-L) | (Ab5-H3) | (Ab5-L) | |
| TAVO3334 × 7378 | EAC33 | EAC34 | EAC73 | EAC78 | IgG1 |
| | (ADA-H) | (ADA-L) | (Ab8-H1) | (Ab8-L3) | |
| TAVO3334 × 8083 | EAC33 | EAC34 | EAC80 | EAC83 | IgG1 |
| | (ADA-H) | (ADA-L) | (Ab9-H1) | (Ab9-L1) | |
| TAV011934 × 12032 | EAC119 | EAC34 | EAC120 | EAC32 | IgG1 with L234A, |
| | (ADA-H) | (ADA-L) | (Ab5-H3) | (Ab5-L) | L235A, M428L, |
| | | | | | N434S mutations |
| TAVO11934 × 12178 | EAC119 | EAC34 | EAC121 | EAC78 | IgG1 with L234A, |
| | (ADA-H) | (ADA-L) | (Ab8-H1) | (Ab8-L3) | L235A, M428L, |
| | | | | | N434S mutations |
| TAVO14434 × 14578 | EAC144 | EAC34 | EAC145 | EAC78 | IgG1 with E233P, |
| | (ADA-H) | (ADA-L) | (Ab8-H1) | (Ab8-L3) | L234A, L235A, |
| | | | | | M428L, N434S |
| | | | | | mutations and G236 |
| | | | | | deleted |
| TAVO14434 × 16183 | EAC144 | EAC34 | EAC161 | EAC83 | IgG1 with E233P, |
| | (ADA-H) | (ADA-L) | (Ab9-H1) | (Ab9-L1) | L234A, L235A, |
| | | | | | M428L, N434S |
| | | | | | mutations and G236 |
| | | | | | deleted |
| TAVO129127 × 12178 | EAC129 | EAC127 | EAC121 | EAC78 | IgG1 with L234A, |
| | (ADA-H1) | (ADA-L1) | (Ab8-H1) | (Ab8-L3) | L235A, M428L, |
| | | | | | N434S mutations |
| TAVO130127 × 12178 | EAC130 | EAC127 | EAC121 | EAC78 | IgG1 with L234A, |
| | (ADA-H1X) | (ADA-L1) | (Ab8-H1) | (Ab8-L3) | L235A, M428L, |
| | | | | | N434S mutations |
| TAVO131127 × 12178 | EAC131 | EAC127 | EAC121 | EAC78 | IgG1 with L234A, |
| | (ADA-H2) | (ADA-L1) | (Ab8-H1) | (Ab8-L3) | L235A, M428L, |
| | | | | | N434S mutations |
| TAVO132127 × 12178 | EAC132 | EAC127 | EAC121 | EAC78 | IgG1 with L234A, |
| | (ADA-H2X) | (ADA-L1) | (Ab8-H1) | (Ab8-L3) | L235A, M428L, |
| | | | | | N434S mutations |
| TAVO133127 × 12178 | EAC133 | EAC127 | EAC121 | EAC78 | IgG1 with L234A, |
| | (ADA-H3) | (ADA-L1) | (Ab8-H1) | (Ab8-L3) | L235A, M428L, |
| | | | | | N434S mutations |
| TAVO134127 × 12178 | EAC134 | EAC127 | EAC121 | EAC78 | IgG1 with L234A, |
| | (ADA-H3X) | (ADA-L1) | (Ab8-H1) | (Ab8-L3) | L235A, M428L, |
| | | | | | N434S mutations |
| TAVO135127 × 12178 | EAC135 | EAC127 | EAC121 | EAC78 | IgG1 with L234A, |
| | (ADA-H4) | (ADA-L1) | (Ab8-H1) | (Ab8-L3) | L235A, M428L, |
| | | | | | N434S mutations |
| TAVO136127 × 12178 | EAC136 | EAC127 | EAC121 | EAC78 | IgG1 with L234A, |
| | (ADA-H4X) | (ADA-L1) | (Ab8-H1) | (Ab8-L3) | L235A, M428L, |
| | | | | | N434S mutations |
| TAVO129128 × 12178 | EAC129 | EAC128 | EAC121 | EAC78 | IgG1 with L234A, |
| | (ADA-H1) | (ADA-L2) | (Ab8-H1) | (Ab8-L3) | L235A, M428L, |
| | | | | | N434S mutations |
| TAVO130128 × 12178 | EAC130 | EAC128 | EAC121 | EAC78 | IgG1 with L234A, |
| | (ADA-H1X) | (ADA-L2) | (Ab8-H1) | (Ab8-L3) | L235A, M428L, |
| | | | | | N434S mutations |
| TAVO131128 × 12178 | EAC131 | EAC128 | EAC121 | EAC78 | IgG1 with L234A, |
| | (ADA-H2) | (ADA-L2) | (Ab8-H1) | (Ab8-L3) | L235A, M428L, |
| | | | | | N434S mutations |
| TAVO132128 × 12178 | EAC132 | EAC128 | EAC121 | EAC78 | IgG1 with L234A, |
| | (ADA-H2X) | (ADA-L2) | (Ab8-H1) | (Ab8-L3) | L235A, M428L, |
| | | | | | N434S mutations |
| TAVO133128 × 12178 | EAC133 | EAC128 | EAC121 | EAC78 | IgG1 with L234A, |
| | (ADA-H3) | (ADA-L2) | (Ab8-H1) | (Ab8-L3) | L235A, M428L, |
| | | | | | N434S mutations |
| TAVO134128 × 12178 | EAC134 | EAC128 | EAC121 | EAC78 | IgG1 with L234A, |
| | (ADA-H3X) | (ADA-L2) | (Ab8-H1) | (Ab8-L3) | L235A, M428L, |
| | | | | | N434S mutations |
| TAVO135128 × 12178 | EAC135 | EAC128 | EAC121 | EAC78 | IgG1 with L234A, |
| | (ADA-H4) | (ADA-L2) | (Ab8-H1) | (Ab8-L3) | L235A, M428L, |
| | | | | | N434S mutations |
| TAVO136128 × 12178 | EAC136 | EAC128 | EAC121 | EAC78 | IgG1 with L234A, |
| | (ADA-H4X) | (ADA-L2) | (Ab8-H1) | (Ab8-L3) | L235A, M428L, |
| | | | | | N434S mutations |
| TAVO166127 × 14578 | EAC166 | EAC127 | EAC145 | EAC78 | IgG1 with E233P, |
| | (ADA-H1X) | (ADA-L1) | (Ab8-H1) | (Ab8-L3) | L234A, L235A, |
| | | | | | M428L, N434S |
| | | | | | mutations and G236 |
| | | | | | deleted |
| TAVO167127 × 14578 | EAC167 | EAC127 | EAC145 | EAC78 | IgG1 with E233P, |
| | (ADA-H2X) | (ADA-L1) | (Ab8-H1) | (Ab8-L3) | L234A, L235A, |
| | | | | | M428L, N434S |
| | | | | | mutations and G236 |
| | | | | | deleted |
| TAVO168127 × 14578 | EAC168 | EAC127 | EAC145 | EAC78 | IgG1 with E233P, |
| | (ADA-H3X) | (ADA-L1) | (Ab8-H1) | (Ab8-L3) | L234A, L235A, |
| | | | | | M428L, N434S |
| | | | | | mutations and G236 |
| | | | | | deleted |
| TAVO169127 × 14578 | EAC169 | EAC127 | EAC145 | EAC78 | IgG1 with E233P, |
| | (ADA-H4X) | (ADA-L1) | (Ab8-H1) | (Ab8-L3) | L234A, L235A, |
| | | | | | M428L, N434S |
| | | | | | mutations and G236 |
| | | | | | deleted |
| TAVO166128 × 14578 | EAC166 | EAC128 | EAC145 | EAC78 | IgG1 with E233P, |
| | (ADA-H1X) | (ADA-L2) | (Ab8-H1) | (Ab8-L3) | L234A, L235A, |
| | | | | | M428L, N434S |
| | | | | | mutations and G236 |
| | | | | | deleted |
| TAVO167128 × 14578 | EAC167 | EAC128 | EAC145 | EAC78 | IgG1 with E233P, |
| | (ADA-H2X) | (ADA-L2) | (Ab8-H1) | (Ab8-L3) | L234A, L235A, |
| | | | | | M428L, N434S |
| | | | | | mutations and G236 |
| | | | | | deleted |
| TAVO168128 × 14578 | EAC168 | EAC128 | EAC145 | EAC78 | IgG1 with E233P, |
| | (ADA-H3X) | (ADA-L2) | (Ab8-H1) | (Ab8-L3) | L234A, L235A, |
| | | | | | M428L, N434S |
| | | | | | mutations and G236 |
| | | | | | deleted |
| TAVO169128 × 14578 | EAC169 | EAC128 | EAC145 | EAC78 | IgG1 with E233P, |
| | (ADA-H4X) | (ADA-L2) | (Ab8-H1) | (Ab8-L3) | L234A, L235A, |
| | | | | | M428L, N434S |
| | | | | | mutations and G236 |
| | | | | | deleted |

### Composition of Anti-TNFα and IL-1β Bispecific Antibody

The anti-TNFα and IL-1β bispecific antibody of the present disclosure encompasses antigen-binding fragments that retain the ability to specifically bind to both TNFα and IL-1β. The antigen binding fragments as used herein may include any 3 or more contiguous amino acids (e.g., 4 or more, 5 or more 6 or more, 8 or more, or even 10 or more contiguous amino acids) of the antibody and encompasses Fab, Fab′, F(ab′)2, and F(v) fragments, or the individual light or heavy chain variable regions or portion thereof. These fragments lack the F_{c} fragment of an intact antibody, clear more rapidly from the circulation, and can have less non-specific tissue binding than an intact antibody. These fragments can be produced from intact antibodies using well known methods, for example by proteolytic cleavage with enzymes such as papain (to produce Fab fragments) or pepsin (to produce F(ab′)2 fragments).

The TNFα and IL-1β binding fragments may also encompass domain antibody (dAb) fragments which consist of a V_{H} domain of heavy chain antibodies (HCAb). Exceptions to the H2L2 structure of conventional antibodies occur in some isotypes of the immunoglobulins found in camelids. Functional VHHs may be obtained by proteolytic cleavage of HCAb of an immunized camelid, by direct cloning of VHH genes from B-cells of an immunized camelid resulting in recombinant VHHs, or from naïve or synthetic libraries. VHHs with desired antigen specificity may also be obtained through phage display methodology.

The TNFα and IL-1β binding fragments may also encompass diabodies, which are bivalent antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites. The TNFα and IL-1β binding fragments may also encompass single-chain antibody fragments (scFv) that bind to both TNFα and IL-1β. An scFv comprises an antibody heavy chain variable region (V_{H}) operably linked to an antibody light chain variable region (V_{L}) wherein the heavy chain variable region and the light chain variable region, together or individually, form a binding site that binds TNFα and IL-1β. Such TNFα and IL-1β binding fragments can be prepared by methods known in the art such as, for example, the synthesis or PCR mediated amplification of the variable portions of the heavy and light chains of an antibody molecule and a flexible protein linker composed of the amino acids Gly and Ser. The resulting DNA fragment is cloned for expression in E. coli or mammalian cells. The expressed TNFα and IL-1β binding fragments are purified from the host cells.

The TNFα and IL-1β binding antibodies and fragments of the present disclosure encompass full length antibody comprising two heavy chains and two light chains. The TNFα and IL-1β binding antibodies can be human or humanized antibodies. Humanized antibodies include chimeric antibodies and CDR-grafted antibodies. Chimeric antibodies are antibodies that include a non-human antibody variable region linked to a human constant region. CDR-grafted antibodies are antibodies that include the CDRs from a non-human “donor” antibody linked to the framework region from a human “recipient” antibody.

Exemplary human or humanized antibodies include IgG, IgM, IgE, IgA, and IgD antibodies. The present antibodies can be of any class (IgG, IgM, IgE, IgGA, IgD, etc.) or isotype and can comprise a kappa or lambda light chain. For example, a human antibody can comprise an IgG F_{c} domain, such as at least one of isotypes, IgG₁, IgG₂, IgG₃ or IgG₄.

In some instances, an IgG F_{c} domain comprises an amino acid sequence having at least 70%, at least 75%, at least 80%, at least 85%, at least 90%, at least 95%, at least 98%, at least 99%, or 100%, amino acid sequence identity to an IgG₁ F_{c} sequence as SEQ ID NO: 47.

In some instances, an IgG F_{c} domain comprises an amino acid sequence having at least 70%, at least 75%, at least 80%, at least 85%, at least 90%, at least 95%, at least 98%, at least 99%, or 100%, amino acid sequence identity to an IgG₂ F_{c} sequence as SEQ ID NO: 48.

In some instances, an IgG F_{c} domain comprises an amino acid sequence having at least 70%, at least 75%, at least 80%, at least 85%, at least 90%, at least 95%, at least 98%, at least 99%, or 100%, amino acid sequence identity to an IgG₃ F_{c} sequence as SEQ ID NO: 49.

In some instances, an IgG F_{c} domain comprises an amino acid sequence having at least 70%, at least 75%, at least 80%, at least 85%, at least 90%, at least 95%, at least 98%, at least 99%, or 100%, amino acid sequence identity to an IgG₄ F_{c} sequence as SEQ ID NO: 50.

A S228P mutation may be made into IgG4 antibodies to enhance IgG₄ stability.

The present anti-TNFα and IL-1β bispecific antibodies may comprise with a modified F_{c} region, wherein the modified F_{c} region comprises at least one amino acid modification relative to a wild-type F_{c} region. In some embodiments, the present anti-TNFα and IL-1β bispecific antibodies are provided with a modified F_{c} region where a naturally-occurring F_{c} region is modified to extend the half-life of the antibody when compared to the parental wild-type antibody in a biological environment, for example, the serum half-life or a half-life measured by an in vitro assay.

Exemplary mutations that may be made singularly or in combination are T250Q, M252Y, I253A, S254T, T256E, P257I, T307A, D376V, E380A, M428L, H433K, N434S, N434A, N434H, N434F, H435A and H435R mutations.

In certain embodiments, the extension of half-life can be realized by engineering the M252Y/S254T/T256E mutations in IgG1 F_{c} as SEQ ID NO: 51, residue numbering according to the EU Index (Dall'Acqua, Kiener et al. 2006).

In certain embodiments, the extension of half-life can also be realized by engineering the M428L/N434S mutations in IgG₁ F_{c} as SEQ ID NO: 52 (Zalevsky, Chamberlain et al. 2010).

In certain embodiments, the extension of half-life can also be realized by engineering the T250Q/M428L mutations in IgG₁ F_{c} as SEQ ID NO: 53 (Hinton, Xiong et al. 2006).

In certain embodiments, the extension of half-life can also be realized by engineering the N434A mutations in IgG₁ F_{c} as SEQ ID NO: 54 (Shields, Namenuk et al. 2001).

In certain embodiments, the extension of half-life can also be realized by engineering the T307A/E380A/N434A mutations in IgG₁ F_{c} as SEQ ID NO: 55 (Petkova, Akilesh et al. 2006).

The effect F_{c} engineering on the extension of antibody half-life can be evaluated in PK studies in mice relative to antibodies with native IgG F_{c}.

In some embodiments, the present anti-TNFα and IL-1β bispecific antibodies are provided with a modified F_{c} region where a naturally-occurring F_{c} region is modified to enhance the antibody resistant to proteolytic degradation by a protease that cleaves the wild-type antibody between or at residues 222-237 (EU numbering).

In certain embodiments, the resistance to proteolytic degradation can be realized by engineering E233P/L234A/L235A mutations in the hinge region with G236 deleted when compared to a parental wild-type antibody as SEQ ID NO: 56, residue numbering according to the EU Index (Kinder, Greenplate et al. 2013).

In instances where effector functionality is not desired, the antibodies of the disclosure may further be engineered to introduce at least one mutation in the antibody F_{c} that reduces binding of the antibody to an activating F_{c}γ receptor (F_{c}γR) and/or reduces F_{c} effector functions such as C1q binding, complement dependent cytotoxicity (CDC), antibody-dependent cell-mediated cytotoxicity (ADCC) or phagocytosis (ADCP).

F_{c} positions that may be mutated to reduce binding of the antibody to the activating F_{c}γR and subsequently to reduce effector functions are those described for example in (Xu, Alegre et al. 2000) (Vafa, Gilliland et al. 2014) (Bolt, Routledge et al. 1993) (Chu, Vostiar et al. 2008) (Shields, Namenuk et al. 2001). Fc mutations with minimal ADCC, ADCP, CDC, Fc mediated cellular activation have been described also as sigma mutations for IgG1, IgG2 and IgG4 (Tam, McCarthy et al. 2017).

Exemplary mutations that may be made singularly or in combination are K214T, E233P, L234V, L234A, deletion of G236, V234A, F234A, L235A, G237A, P238A, P238S, D265A, S267E, H268A, H268Q, Q268A, N297A, A327Q, P329A, D270A, Q295A, V309L, A327S, L328F, A330S and P331S mutations on IgG₁, IgG₂, IgG₃ or IgG₄.

Exemplary combination mutations that may be made to reduced ADCC are L234A/L235A on IgG₁, V234A/G237A/P238S/H268A/V309L/A330S/P331S on IgG₂, F234A/L235A on IgG₄, S228P/F234A/L235A on IgG₄, N297A on IgG₁, IgG₂, IgG₃ or IgG₄, V234A/G237A on IgG₂, K214T/E233P/L234V/L235A/G236-deleted/A327G/P331A/D365E/L358M on IgG₁, H268Q/V309L/A330S/P331S on IgG₂, S267E/L328F on IgG₁, L234F/L235E/D265A on IgG₁, L234A/L235A/G237A/P238S/H268A/A330S/P331S on IgG₁, S228P/F234A/L235A/G237A/P238S on IgG₄, and S228P/F234A/L235A/G236-deleted/G237A/P238S on IgG₄. Hybrid IgG_{2/4}F_{c} domains may also be used, such as F_{c} with residues 117-260 from IgG₂ and residues 261-447 from IgG₄.

Antibodies of the disclosure further comprising conservative modifications are within the scope of the disclosure.

“Conservative modifications” refer to amino acid modifications that do not significantly affect or alter the binding characteristics of the antibody containing the amino acid sequences. Conservative modifications include amino acid substitutions, additions and deletions. Conservative substitutions are those in which the amino acid is replaced with an amino acid residue having a similar side chain. The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), amide (e.g., asparagine, glutamine), beta-branched side chains (e.g., threonine, valine, isoleucine) and sulfur-containing side chains (cysteine, methionine). Furthermore, any native residue in the polypeptide may also be substituted with alanine, as has been previously described for alanine scanning mutagenesis. Amino acid substitutions to the antibodies of the disclosure may be made by known methods for example by PCR mutagenesis (U.S. Pat. No. 4,683,195). Alternatively, libraries of variants may be generated for example using random (NNK) or non-random codons, for example DVK codons, which encode 11 amino acids (Ala, Cys, Asp, Glu, Gly, Lys, Asn, Arg, Ser, Tyr, Trp). The resulting antibody variants may be tested for their characteristics using assays described herein.

The antibodies of the disclosure may be post-translationally modified by processes such as glycosylation, isomerization, deglycosylation or non-naturally occurring covalent modification such as the addition of polyethylene glycol moieties (pegylation) and lipidation. Such modifications may occur in vivo or in vitro. For example, the antibodies of the disclosure may be conjugated to polyethylene glycol (PEGylated) to improve their pharmacokinetic profiles. Conjugation may be carried out by techniques known to those skilled in the art. Conjugation of therapeutic antibodies with PEG has been shown to enhance pharmacodynamics while not interfering with function.

Antibodies of the disclosure may be modified to improve stability, selectivity, cross-reactivity, affinity, immunogenicity or other desirable biological or biophysical property are within the scope of the disclosure. Stability of an antibody is influenced by a number of factors, including (1) core packing of individual domains that affects their intrinsic stability, (2) protein/protein interface interactions that have impact upon the HC and LC pairing, (3) burial of polar and charged residues, (4) H-bonding network for polar and charged residues; and (5) surface charge and polar residue distribution among other intra- and inter-molecular forces (Worn and Pluckthun 2001). Potential structure destabilizing residues may be identified based upon the crystal structure of the antibody or by molecular modelling in certain cases, and the effect of the residues on antibody stability may be tested by generating and evaluating variants harboring mutations in the identified residues. One of the ways to increase antibody stability is to raise the thermal transition midpoint (Tm) as measured by differential scanning calorimetry (DSC). In general, the protein Tm is correlated with its stability and inversely correlated with its susceptibility to unfolding and denaturation in solution and the degradation processes that depend on the tendency of the protein to unfold. A number of studies have found correlation between the ranking of the physical stability of formulations measured as thermal stability by DSC and physical stability measured by other methods. Formulation studies suggest that a Fab Tₘ has implication for long-term physical stability of a corresponding mAb.

Antibodies of the disclosure may have amino acid substitutions in the F_{c} region that improve manufacturing and drug stability. An example for IgG₁ is H224S (or H224Q) in the hinge 221-DKTHTC-226 (Eu numbering) which blocks radically induced cleavage; and for IgG₄, the S228P mutation blocks half-antibody exchange.

Antibodies of the disclosure may comprise additional amino acid sequences that can function as an inhibitory domain to mask the antibodies in the recognition and binding to their antigens and hence the antibodies exist as inactive or pro-antibodies. The pro-antibodies can be converted into active antibodies with the removal of the inhibitory domain sequences by for example site-specific proteases. The inactive pro-antibodies may have reduced toxicity systematically but can be activated at the disease sites abundant in proteases for therapeutic effects.

### Generation of Anti-TNFα and IL-1β Bispecific Antibody

The bispecific antibody is generated by a process known as controlled F_{ab} arm exchange from two parental antibodies with F405L and K409R (EU numbering) mutation in IgG Fc respectively (Labrijn, Meesters et al. 2014). The controlled F_{ab} arm exchange reaction is the result of a disulfide-bond isomerization reaction and dissociation-association of C_{H3} domains. First, two parental antibodies are generated, one bearing the F405L F_{c} mutation, and one bearing the K409R F_{c} mutation. The heavy chain disulfide bonds in the hinge regions of the parental antibodies are reduced and the heavy chains of the parental antibodies are separated. The F405L and K409R mutations favor heterodimerization over homodimerization of the heavy chains. Therefore, the resulting free cysteines of one of the parental antibodies form an inter heavy-chain disulfide bond with cysteine residues of a second parental antibody. The resulting product is a heterodimerized antibody with one half coming from one parental antibody and the other half coming from another parental antibody.

In the present disclosure, the bispecific antibody with dual specificity to both TNFα and IL-1β is generated from one parental antibody to TNFα with F405L Fc mutation and another parental antibody to IL-1β with K409R Fc mutation by controlled Fab arm exchange.

The F405L and K409R mutations on the parental antibodies of the present disclosure can be engineered on a human F_{c}, a non-human primate F_{c}, a murine F_{c} domain, and the like. The F405L and K409R mutations on the parental antibodies of the present disclosure can be engineered on a human IgG₁ F_{c}, a human IgG₂ F_{c}, a human IgG₃ F_{c}, a human IgG₄ F_{c}, etc.

In some instances, an F_{c} domain with the F405L mutation comprises an amino acid sequence having at least 70%, at least 75%, at least 80%, at least 85%, at least 90%, at least 95%, at least 98%, at least 99%, or 100%, amino acid sequence identity to an IgG₁ F_{c} with the F405L mutation as SEQ ID NO: 57.

In some instances, an F_{c} domain with the K409R mutation comprises an amino acid sequence having at least 70%, at least 75%, at least 80%, at least 85%, at least 90%, at least 95%, at least 98%, at least 99%, or 100%, amino acid sequence identity to an IgG₁ F_{c} with the K409R mutation as SEQ ID NO: 58.

The anti-TNFα×IL1β bispecific antibody of the present disclosure may be generated by other F_{c} mutations and engineering processes that facilitate F_{c} heterodimerization, including, but not limited to, Knob-in-Hole and the electrostatically-matched interactions.

In the Knob-in-Hole strategy (see, e.g., Intl. Publ. No. WO 2006/028936, incorporated by reference), selected amino acids forming the interface of the C_{H3} domains in human IgG can be mutated at positions affecting C_{H3} domain interactions to promote heterodimer formation. An amino acid with a small side chain (hole) is introduced into one F_{c} domain and an amino acid with a large side chain (knob) is introduced into the other F_{c} domain of the parental antibodies. After co-expression of the two heavy chains, a heterodimer is formed because of the preferential interaction of the heavy chain with a “hole” with the heavy chain with a “knob.” Exemplary C_{H3} substitution pairs forming a knob and a hole include: T366Y/F405A, T366W/F405W, F405W/Y407A, T394W/Y407T, T394S/Y407A, T366W/T394S, F405W/T394S and T366W/T366S_L368A_Y407V.

In the electrostatically-matched interactions strategy, mutations can be engineered to generate positively charged residues at one C_{H3} surface and negatively charged residues at a second C_{H3} surface as described in US 2010/0015133 A1; US 2009/0182127 A1; US 2010/028637 A1, or US 2011/0123532 A1. Heterodimerization of heavy chain can be formed by electrostatically-matched interactions between two mutated F_{c}.

The formation of bispecific antibody can be assessed by an ELISA assay. In the present disclosure, IL-1β is coated on the ELISA plate and then the bispecific antibody and TNFα are added. After washing the non-specific binding, the presence of TNFα is detected by an anti-TNFα antibody followed by a HRP-conjugated secondary antibody. The formation of bispecific antibody is reflected by the ELISA signal since only the bispecific antibody is capable of binding TNFα and IL1β simultaneously with both arms.

The formation of bispecific antibody can also be assessed by analytical HPLC if there is a detectable difference in the biophysical properties of the two parental antibodies. A difference in pI may leads to two separate peaks for the two parental antibodies on Cation Exchange chromatography and the bispecific antibody may migrate as a peak in between. A difference in hydrophobicity may leads to two separate peaks for the two parental antibodies on hydrophobic interaction chromatography and the bispecific antibody may migrate as a peak in between. The analytical HPLC not only demonstrates the formation of bispecific antibody, but also allows the quantitation of percentage of bispecific antibody formed.

### Expression and Purification of the Parental Anti-TNFα and Anti-IL-1β Antibodies

The anti-TNFα and anti-IL-1β parental antibodies and fragments of the disclosure can be encoded by a single nucleic acid (e.g., a single nucleic acid comprising nucleotide sequences that encode the light and heavy chain polypeptides of the antibody), or by two or more separate nucleic acids, each of which encode a different part of the antibody or antibody fragment. The nucleic acids can be inserted into vectors, e.g., nucleic acid expression vectors and/or targeting vectors. Such vectors can be used in various ways, e.g., for the expression of anti-TNFα and anti-IL-1β binding antibody or antibody fragment in a cell or transgenic animal. Vectors are typically selected to be functional in the host cell in which the vector will be used. A nucleic acid molecule encoding anti-TNFα and anti-IL-1β binding antibody or fragment may be amplified/expressed in prokaryotic, yeast, insect (baculovirus systems) and/or eukaryotic host cells. Selection of the host cell will depend in part on whether the anti-TNFα and anti-IL-1β binding antibody or fragment is to be post-translationally modified (e.g., glycosylated and/or phosphorylated). If so, yeast, insect, or mammalian host cells are preferable. Expression vectors typically contain one or more of the following components: a promoter, one or more enhancer sequences, an origin of replication, a transcriptional termination sequence, a complete intron sequence containing a donor and acceptor splice site, a leader sequence for secretion, a ribosome binding site, a polyadenylation sequence, a polylinker region for inserting the nucleic acid encoding the polypeptide to be expressed, and a selectable marker element.

As non-limiting example, the disclosure provides for polynucleotides comprising the polynucleotide sequences of SEQ ID NOs: 59, 60, 61, or 62, encoding the anti-TNFα antibody heavy chain EAC33, anti-TNFα antibody light chain EAC34, anti-IL-1β antibody heavy chain EAC53 and anti-IL-1β antibody light chain EAC32, respectively.

In most cases, a leader or signal sequence is engineered at the N-terminus of the anti-TNFα and anti-IL-1β antibodies or fragments to guide its secretion. The secretion of anti-TNFα and anti-IL-1β antibodies or fragments from a host cell will result in the removal of the signal peptide from the antibody or fragment. Thus, the mature antibody or fragment will lack any leader or signal sequence. In some cases, such as where glycosylation is desired in a eukaryotic host cell expression system, one may manipulate the various presequences to improve glycosylation or yield. For example, one may alter the peptidase cleavage site of a signal peptide, or add prosequences, which also may affect glycosylation.

The disclosure further provides a cell (e.g., an isolated or purified cell) comprising a nucleic acid or vector of the disclosure. The cell can be any type of cell capable of being transformed with the nucleic acid or vector of the disclosure so as to produce a polypeptide encoded thereby. To express the anti-TNFα and anti-IL-1β binding antibodies or fragments, DNAs encoding partial or full-length light and heavy chains, obtained as described above, are inserted into expression vectors such that the genes are operatively linked to transcriptional and translational control sequences.

Methods of introducing nucleic acids and vectors into isolated cells and the culture and selection of transformed host cells in vitro are known in the art and include the use of calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, and electroporation.

After introducing the nucleic acid or vector of the disclosure into the cell, the cell is cultured under conditions suitable for expression of the encoded sequence. The antibody, antigen binding fragment, or portion of the antibody then can be isolated from the cell.

In certain embodiments, two or more vectors that together encode anti-TNFα and anti-IL-1β binding antibodies, or antigen binding fragments thereof, can be introduced into the cell.

Purification of anti-TNFα and anti-IL-1β binding antibodies or fragments which have been secreted into the cell media can be accomplished using a variety of techniques including affinity, immunoaffinity or ion exchange chromatography, molecular sieve chromatography, preparative gel electrophoresis or isoelectric focusing, chromatofocusing, and high-pressure liquid chromatography. For example, antibodies comprising a F_{c} region may be purified by affinity chromatography with Protein A, which selectively binds the F_{c} region.

Modified forms of an antibody or antigen binding fragment may be prepared with affinity tags, such as hexahistidine or other small peptide such as FLAG or myc at either its carboxyl or amino terminus and purified by a one-step affinity column. For example, polyhistidine binds with great affinity and specificity to nickel, thus an affinity column of nickel (such as the Qiagen® nickel columns) can be used for purification of polyhistidine-tagged selective binding agents. In some instances, more than one purification step may be employed.

### Binding and Functional Activity of Anti-TNFα and IL-1β Bispecific Antibody

The present disclosure encompasses anti-TNFα and IL-1β bispecific antibodies that bind selectively to TNFα and IL-1β in that they bind to TNFα and IL-1β with greater affinity than to other antigens. The anti-TNFα and IL-1β bispecific antibodies and fragments may bind selectively to human TNFα and IL-1β, but also bind detectably to non-human TNFα and IL-1β. For example, the antibodies or fragments may bind to one or more of rodent TNFα and IL-1β, primate TNFα and IL-1β, dog TNFα and IL-1β, and rabbit TNFα and IL-1β, or guinea pig TNFα and IL-1β. Alternatively or additionally, the TNFα and IL-1β binding antibodies may have the same or substantially the same potency against recombinant human TNFα and IL-1β and endogenous human TNFα and IL-1β.

In vitro and cell-based assays are well described in the art for use in determining binding of TNFα and IL-1β to their receptors. For example, the binding of TNFα and IL-1β to their receptors may be determined by immobilizing an TNFα and IL-1β binding antibody, sequestering TNFα and IL-1β with the immobilized antibody and determining whether the TNFα and IL-1β is bound to the antibody, and contacting a soluble form of receptor with the bound TNFα and IL-1β/antibody complex and determining whether the soluble receptor is bound to the complex. The protocol may also include contacting the soluble receptors with the immobilized antibody before the contact with TNFα and IL-1β, to confirm that the soluble receptor does not bind to the immobilized antibody. This protocol can be performed using a Biacore® instrument for kinetic analysis of binding interactions. Such a protocol can also be employed to determine whether an antibody or other molecule permits or blocks the binding of TNFα and IL-1β to their receptors.

For other binding assays, the permitting or blocking of TNFα and IL-1β binding to their receptors may be determined by comparing the binding of TNFα and IL-1β to receptors in the presence or absence of TNFα and IL-1β antibodies. Blocking is identified in the assay readout as a designated reduction of TNFα and IL-1β binding to receptors in the presence of anti-TNFα and IL-1β antibodies, as compared to a control sample that contains the corresponding buffer or diluent but not an anti-TNFα and IL-1β antibody. The assay readout may be qualitatively viewed as indicating the presence or absence of blocking, or may be quantitatively viewed as indicating a percent or fold reduction in binding due to the presence of the antibody or fragment. when an TNFα and IL-1β binding bispecific antibody substantially blocks TNFα and IL-1β binding to receptor, the TNFα and IL-1β binding to receptor is reduced by at least 10-fold, alternatively at least about 20-fold, alternatively at least about 50-fold, alternatively at least about 100-fold, alternatively at least about 1000-fold, alternatively at least about 10000-fold, or more, compared to the same concentrations of TNFα and IL-1β binding to receptors in the absence of the antibody or fragment.

Preferred anti-TNFα and IL-1β bispecific antibodies for use in accordance with the disclosure generally bind to human TNFα and IL-1β with high affinity (e.g., as determined with BIACORE), such as for example with an equilibrium binding dissociation constant (K_{D}) for TNFα and IL-1β of about 10 nM or less, about 5 nM or less, about 1 nM or less, about 500 pM or less, or more preferably about 250 pM or less, about 100 pM or less, about 50 pM or less, about 25 pM or less, about 10 pM or less, about 5 pM or less, about 3 pM or less about 1 pM or less, about 0.75 pM or less, about 0.5 pM or less, or about 0.3 pM or less.

Antibodies or fragments of the present disclosure may, for example, bind to TNFα and IL-1β with an EC50 of about 10 nM or less, about 5 nM or less, about 2 nM or less, about 1 nM or less, about 0.75 nM or less, about 0.5 nM or less, about 0.4 nM or less, about 0.3 nM or less, or even about 0.2 nM or less, as determined by enzyme linked immunosorbent assay (ELISA).

Preferably, the antibody or antibody fragment of the present disclosure does not cross-react with any target other than TNFα and IL-1β. For example, the present antibodies and fragments may bind to IL-1β, but do not detectably bind to IL-la, or have at least about 100 times (e.g., at least about 150 times, at least about 200 times, or even at least about 250 times) greater selectivity in its binding of IL-1β relative to its binding of IL-la.

The present disclosure also encompasses neutralizing antibodies or neutralizing fragments thereof which bind to TNFα and IL-1β so as to neutralize biological activity of the TNFα and IL-1β. Neutralization of biological activity of TNFα and IL-1β can be assessed by assays for one or more indicators of TNFα and IL-1β biological activity, such as TNFα and IL-1β stimulated reporter gene expression in a reporter assay, TNFα and IL-1β stimulated release of IL-6 from human fibroblasts or other cells, TNFα and IL-1β induced proliferation of T helper cells. Neutralization of biological activity of TNFα and IL-1β can also be assessed in vivo by mouse arthritis models. Preferably the TNFα and IL-1β binding antibodies and fragments of the present disclosure neutralize the biological activity of TNFα and IL-1β connected with the signalling function of their receptors bound by the TNFα and IL-1β.

The present antibodies or fragments may be neutralizing antibodies or fragments which bind specifically to TNFα and IL-1β epitope that affects biological activity of TNFα and IL-1β. The present antibodies or fragments can bind to a neutralization-sensitive epitope of TNFα and IL-1β. When a neutralization-sensitive epitope of TNFα and IL-1β is bound by one of the present antibodies or fragments, the result is a loss of biological activity of the TNFα and IL-1β containing the epitope.

### Pharmaceutical Compositions

TNFα and IL-1β binding antibodies and antibody fragments for use according to the present disclosure can be formulated in compositions, especially pharmaceutical compositions, for use in the methods herein. Such compositions comprise a therapeutically or prophylactically effective amount of an TNFα and IL-1β binding antibody or antibody fragment of the disclosure in mixture with a suitable carrier, e.g., a pharmaceutically acceptable agent. Typically, TNFα and IL-1β binding antibodies and antibody fragments of the disclosure are sufficiently purified for administration to an animal before formulation in a pharmaceutical composition.

Pharmaceutically acceptable agents include carriers, excipients, diluents, antioxidants, preservatives, coloring, flavoring and diluting agents, emulsifying agents, suspending agents, solvents, fillers, bulking agents, buffers, delivery vehicles, tonicity agents, cosolvents, wetting agents, complexing agents, buffering agents, antimicrobials, and surfactants.

The composition can be in liquid form or in a lyophilized or freeze-dried form and may include one or more lyoprotectants, excipients, surfactants, high molecular weight structural additives and/or bulking agents.

Compositions can be suitable for parenteral administration. Exemplary compositions are suitable for injection or infusion into an animal by any route available to the skilled worker, such as intraarticular, subcutaneous, intravenous, intramuscular, intraperitoneal, intracerebral (intraparenchymal), intracerebroventricular, intramuscular, intraocular, intraarterial, intralesional, intrarectal, transdermal, oral, and inhaled routes.

Pharmaceutical compositions described herein can be formulated for controlled or sustained delivery in a manner that provides local concentration of the product (e.g., bolus, depot effect, topical) sustained release and/or increased stability or half-life in a particular local environment.

### Methods of Use

The present disclosure provides uses of the bispecific anti-TNFα and IL-1β antibodies provided herein to treat patients who would undergo conventional anti-TNFα therapy or anti-IL-1β therapy. Exemplary indications include rheumatoid arthritis, inflammatory bowel disease, and other systemic inflammatory conditions. The bispecific antibody enhances responsiveness and/or minimizes toxicities of each of the anti-cytokine therapy alone. In embodiments, the bispecific antibody may be given at a lower efficacious dose compared to the corresponding monoclonal antibodies, thus minimizing potential toxicity. Besides, the lower dosing combined with infrequent dosing due to the longer half-life of the bispecific anti-TNFα and IL-1β antibody with optimal F_{c} engineering may lead to lower immunogenicity risk so it may take longer time for the development of anti-drug antibodies.

Considerations for use of a dual TNFα and IL-1β inhibitor antibody are obtained from data mining of disease states where both TNFα and IL-1β have a strong presence. Select examples are described below which have high target-disease association with both cytokines (https://www.targetvalidation.org/).

In gout, uric acid has been shown to promote IL-1β secretion in human monocytes. TNFα stimulation was also known to induce pro IL-1β mRNA expression. Yokose et. al., demonstrated that by priming human neutrophils with TNFα, this would promote uric acid mediated IL-1β secretion in gouty joints. These findings thus pointed also to the utility of such dual TNFα and IL-1β inhibition in patients with gouty arthritis (Yokose, Sato et al. 2018).

Post-traumatic arthritis is a common secondary complication to severe joint trauma. As the disease progresses, it may lead to osteoarthritis eventually. In a rabbit animal model of post-traumatic arthritis, Tang et. al., showed that simultaneous silencing of both IL-1β and TNFα (via RNA interference) led to much less cartilage damage and joint degeneration. The co-treated group also showed greater alleviation of symptoms associated with the traumatic joint damage (Tang, Hao et al. 2015). Therefore, post-traumatic arthritis would also be another key indication of this novel bispecific antibody.

Another important potential use of this dual-specificity anti-TNFα and IL-1β bispecific antibody is in wound healing. Angiogenesis is an important step in wound healing and it is affected by the functions of endothelial cells. Cdc42 is known to play a key role in endothelial cell function and vascular development. The depletion of Cdc42 had been found to lead to poor wounding healing by mean of IL-1β and TNF-α increase in the wound bed. By blocking both IL-113 and TNFα simultaneously, it is likely that this would normalize function of Cdc42 and thus potentially hastening the pace of wound healing (Xu, Lv et al. 2019).

In addition, neuropathic pain such as sciatica has been shown to be responsive to anti-TNFα therapy (Hess, Axmann et al. 2011). Older TNF synthesis inhibitors curcumin and thalidomide had also been shown to be effective in reducing neuropathic pain (Li, Zhang et al. 2013). In fact, rheumatoid arthritis patients are known to feel better soon after anti-TNFα therapy long before their joint damage is improved (Taylor 2010). Cytokine IL-1β is also known to be a critical factor in inflammation and neuropathic pain. Therefore, this novel disclosure of a dual-specificity anti-TNFα and IL-1β bispecific antibody would have great potential in managing such condition.

There are many other literature data pointing to the utilities of simultaneous IL-1β and TNFα inhibition. For example, Parkinson's disease was shown to have an elevated component of both IL-1β and TNFα (Leal, Casabona et al. 2013, Erekat and Al-Jarrah 2018). Meanwhile, chronic hepatitis B infection were associated with intense inflammation from the increase of IL-113 and TNFα (Lou, Hou et al. 2013, Wu, Kanda et al. 2016). Therefore, this novel disclosure of a dual-specificity anti-TNFα and IL-1β bispecific antibody may offer a novel therapeutic approach for Parkinson's disease and chronic hepatitis B infection.

Many chemotherapy or cancer targeted therapy have been associated with a condition known as Cancer-Treatment Related Symptoms (CTRS) that are mediated mainly via elevated IL-1β and TNFα. Use of a dual inhibitor to suppress these cytokines such as the current disclosure may have the potential in hastening recovery from the suffering of these patients (Smith, Leo et al. 2014). Lastly, elevation of both TNFα and IL-1β has also been found in breast cancer (Martinez-Reza, Diaz et al. 2019). In fact, inflammatory cytokines, including both TNFα and IL-113, are known to be present in the tumor micro-environment to promote cancer growth and disease progression (Kuratnik, Senapati et al. 2012, Kobayashi, Vali et al. 2016). Modulating both TNFα and IL-1β may likely change the tumor microenvironment. Therefore, this disclosure of a bispecific antibody against both TNFα and IL-1β may also have a role as an adjunct therapy with other standard of care anti-cancer agents in cancer treatment. In addition, the combination use of bispecific antibodies with dual specificities to both TNFα and IL-1β and antibodies to immune-oncology targets, such as PD1, may offer more effective therapeutic efficacies to treat different types of cancer.

To treat neurologic disorders, Fc engineering can be adopted to facilitate the anti-TNFα and IL-1β bispecific antibody with increased affinity to neonatal Fc Receptors (FcRn) which would then allow Ig-Ab transcytosis across the blood-brain barrier (Sockolosky, Tiffany et al. 2012, Xiao and Gan 2013). Likewise, protein fusions that allow facilitative diffusion to these constructs can increase the transport across the blood brain barrier. This would foster the potential for therapeutic antibody-mediated TNFα and IL-1β neutralization within the CNS for inflammatory conditions within the brain such as stroke, Alzheimer's disease, or other chronic neurologic disorders.

In addition to therapeutic uses, the present antibodies and fragments can be used in diagnostic methods to detect TNFα and IL-1β (for example, in a biological sample, such as serum or plasma), using a conventional immunoassay, such as an enzyme linked immunosorbent assays (ELISA), an radioimmunoassay (MA) or tissue immunohistochemistry.

A method for detecting TNFα and IL-1β in a biological sample can comprise the steps of contacting a biological sample with one or more of the present antibodies or fragments and detecting either the antibody or fragment bound to TNFα and IL-1β or unbound antibody or fragment, to thereby detect TNFα and IL-1β in the biological sample. The antibody or fragment can be directly or indirectly labelled with a detectable substance to facilitate detection of the bound or unbound antibody. Suitable detectable substances include various enzymes, prosthetic groups, fluorescent materials, luminescent materials and radioactive materials.

### EXAMPLES

The following examples are provided to describe the present disclosure in greater detail. They are intended to illustrate, not to limit, the present disclosure.

### Example 1: Generation Anti-TNFα and IL-1β Bispecific Antibody

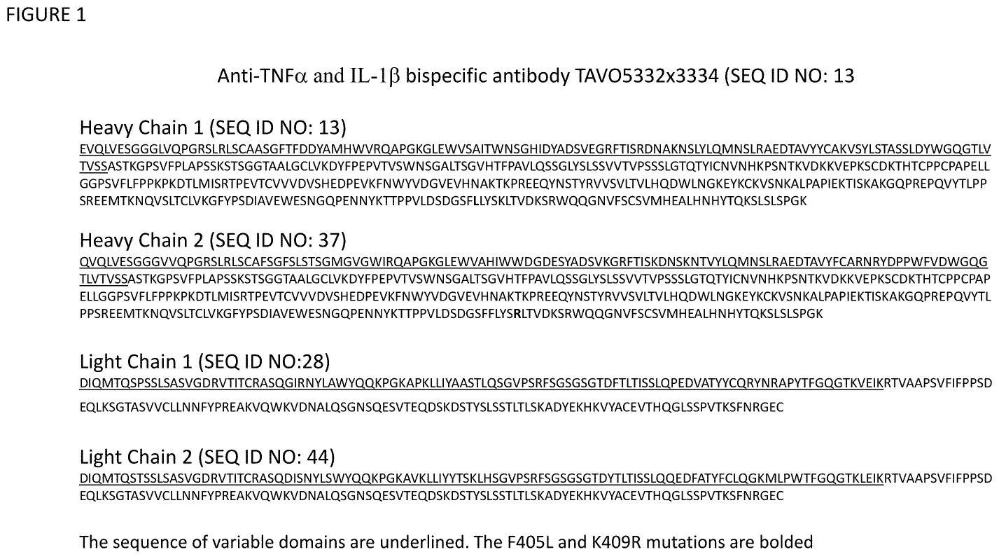

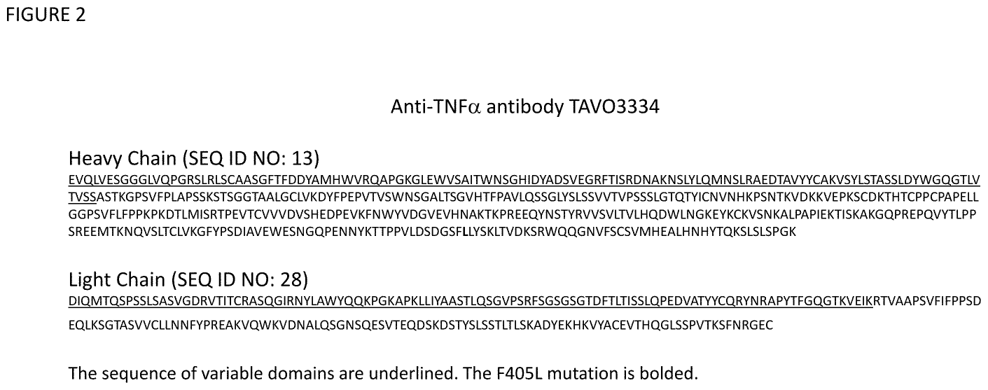

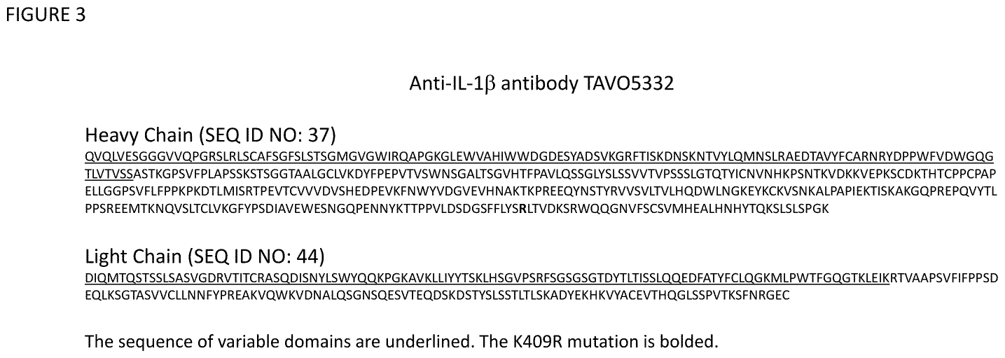

The bispecific antibody against both TNFα and IL-1β, designated as TAVO3334×5332 in this disclosure (FIG. 1), was generated from two parental antibodies, an anti-human TNFα antibody designated as TAVO3334 and an anti-human IL-1β antibody designated as TAVO5332 by a process known as controlled Fab-arm exchange. The anti-human TNFα antibody TAVO3334 has a F405L mutation in its IgG1 Fc (FIG. 2) and the anti-human IL-1β antibody TAVO5332 has a K409R mutation in its IgG1 Fc (FIG. 3) to facilitate the bispecific antibody formation.

To produce the parental antibodies, plasmids encoding heavy chain and light chain of TAVO3334 and TAVO5332 were co-transfected into Expi293F cells following the transfection kit instructions (Thermo Scientific). Cells were spun down five days post transfection, and the supernatant were passed through a 0.2 μm filter. The purification of expressed antibodies in supernatant was carried out by affinity chromatography over protein A agarose column (GE Healthcare Life Sciences). The purified antibodies were buffer-exchanged into DPBS, pH7.2 by dialysis, and protein concentrations were determined by UV absorbance at 280 nm.

For controlled F_{ab}-arm exchange, equal molar amounts of both parental antibodies were mixed together and reduced for 5 hours in the presence of 75 mM 2-mercaptoethylamine (2-MEA). The reaction mixture was dialyzed against DPBS to allow the bispecific antibody formation.

By a similar process, other forms of anti-TNFα and IL-1β bispecific antibodies employed in these examples were also generated by controlled Fab-arm exchange.

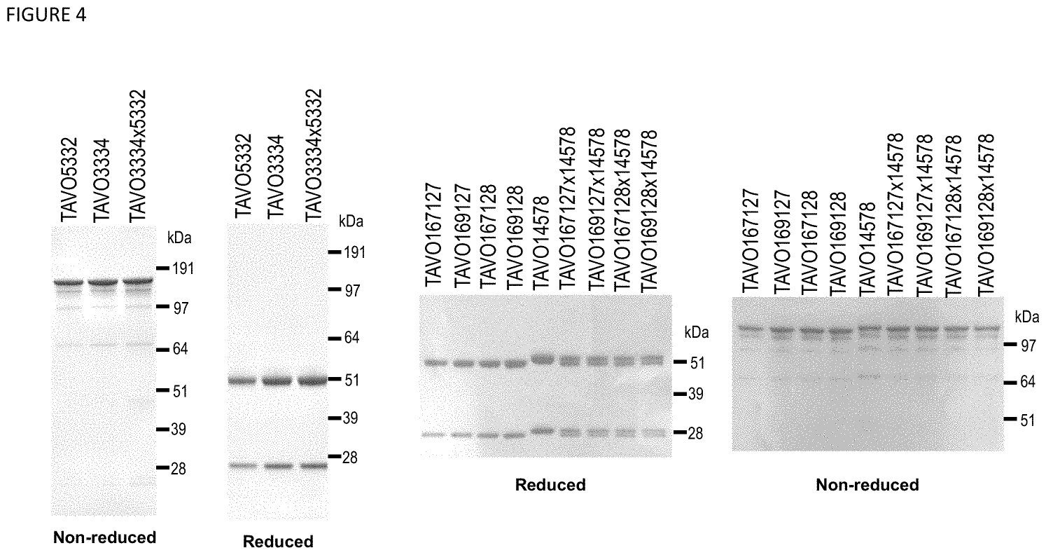

The parental antibodies TAVO5332 and TAVO3334, and the bispecific antibody TAVO3334×5332, were subjected to SDS-PAGE analysis (FIG. 4). Under the reduced condition, all three antibodies had heavy chains and light chains with the expected molecular weight. Under the non-reduced condition, all three antibodies migrated as a major protein band with a molecular weight around 150 kDa. Similar SDS-PAGE analysis was also performed on TAVO167127×14578, TAVO169127×14578, TAVO167128×14578, and TAVO169128×14578, which are anti-TNFα and IL-1β bispecific IgG1 antibodies engineered with E233P, L234A, L235A, F405L, M428L, N434S Fc mutations and with G236 deleted, and the corresponding parental antibodies TAVO167127, TAVO169127, TAVO167128, TAVO169128 and TAVO14578. Expected protein bands were obtained under both reduced and non-reduced conditions, indicating that these extensive F_{c} mutations do not affect structural integrity of these antibodies (FIG. 4).

### Example 2: Confirmation of Formation of Anti-TNFα and IL-1β Bispecific Antibody

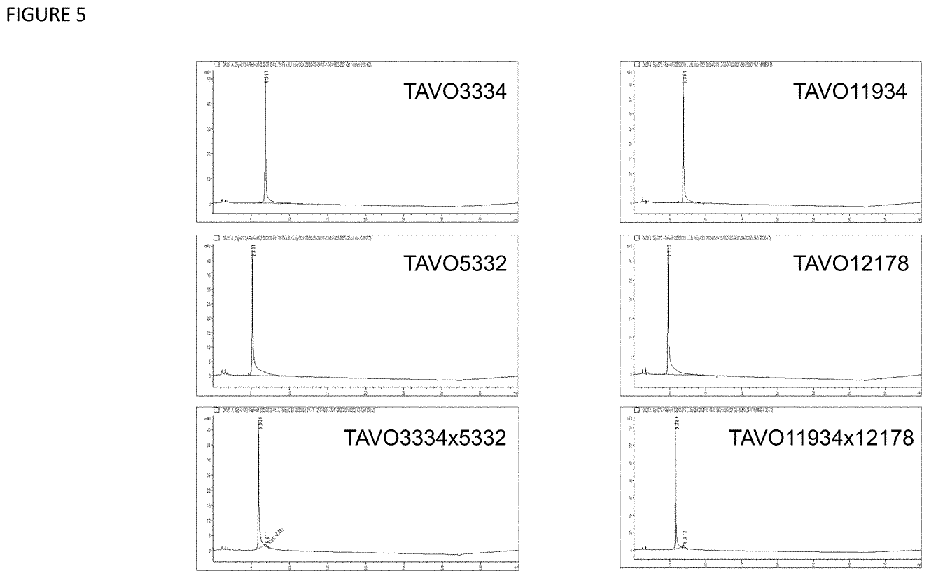

The formation of anti-TNFα and IL-1β bispecific antibody TAVO3334×5332 was assessed by Cation Exchange (CEX) chromatography. 20 μg of antibodies was loaded onto Bio SCX ion exchange column (Agilent). The peak of TAVO3334 appeared at 6.841 minute while TAVO5332 appeared at 5.137 minute (FIG. 5). The peak of main protein band for TAVO3334×5332 migrated at 5.936 minute. Further calculation of the area under the curve (AUC) indicated that 97% of the parental antibodies formed the bispecific antibody by the Fab-arm exchange (FIG. 5).

Similarly, when anti-TNFα and IL-1β bispecific antibody TAVO11934×12178 and related parental antibodies were assessed, the peak of TAVO11934 appeared at 6.861 minute while TAVO12178 appeared at 4.725 minute (FIG. 5). The peak of main protein band for TAVO11934×12178 migrated at 5.783 minute. Further calculation of the AUCindicated that 96% of the parental antibodies formed the TAVO11934×12178 bispecific antibody by the Fab-arm exchange (FIG. 5)

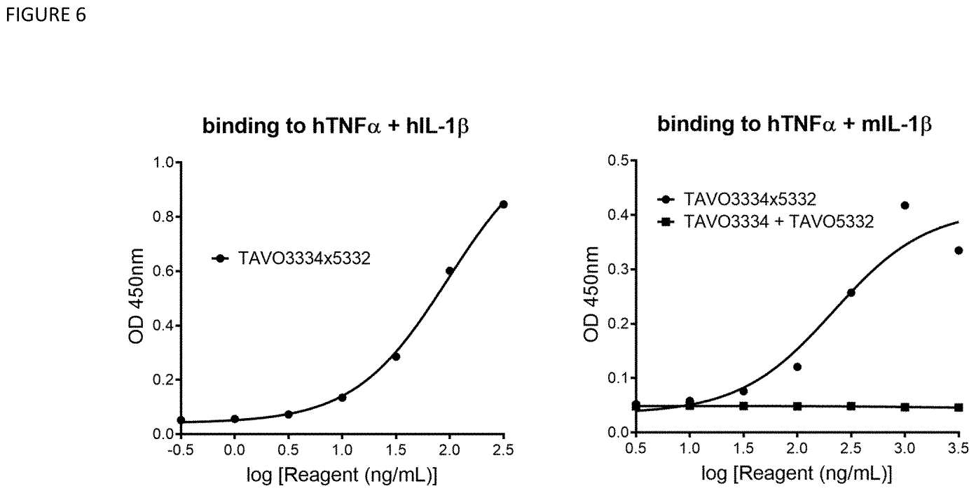

The formation of bispecific antibody was also assessed by an ELISA-based binding assay. In this assay, human IL-1β was coated on the plate and then the bispecific antibody TAVO3334×5332 and TNFα were added. After washing the non-specific binding, the presence of TNFα was detected by an anti-TNFα detection antibody followed by a HRP-conjugated secondary antibody (Biolegend). It was observed that the bispecific antibody TAVO3334×5332 dose-dependently mediated the binding of both TNFα and IL1β (FIG. 6). Similar ELISA assays were also performed by coating mouse IL-1β on the plate. Consistently, dose-dependent recruitment of human TNFα was observed by bispecific antibody TAVO3334×5332, but not by a mixture of the two parental antibodies TAVO3334 and TAVO5332 (FIG. 6). This data suggested the formation of bispecific antibody which is capable of binding TNFα and IL-1β simultaneously with both arms.

### Example 3: Binding Affinity to TNFα and IL-1β of Bispecific Antibody and their Respective Parental Antibodies

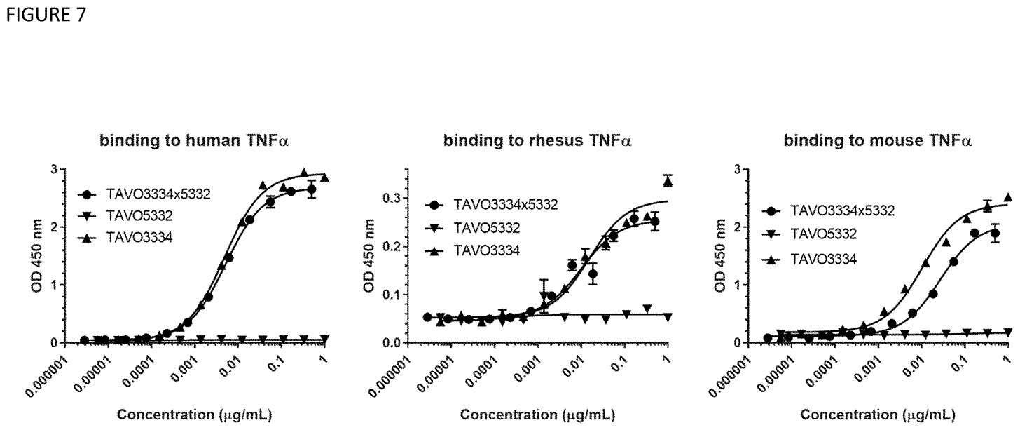

ELISA-based binding assay was employed to evaluate the binding to TNFα and IL1β from different species by the bispecific antibody TAVO3334×5332 and its parent antibodies TAVO5332 and TAVO3334. In this assay, 1 μg/mL recombinant human TNFα or IL-1β (R&D systems) was coated on ELISA plate. Increasing concentrations of TAVO3334×5332, TAVO5332 and TAVO3334 antibodies were applied on the plate and their binding to the recombinant human TNFα or IL-1β were detected by HRP-conjugated anti-human secondary antibody. It was observed that the anti-TNFα and IL-1β bispecific antibody TAVO3334×5332 dose-dependently bound TNFα from human, rhesus monkey and mouse with similar potency as that anti-TNFα antibody TAVO3334, while anti-IL-1β antibody TAVO5332 did not show binding activity (FIG. 7).

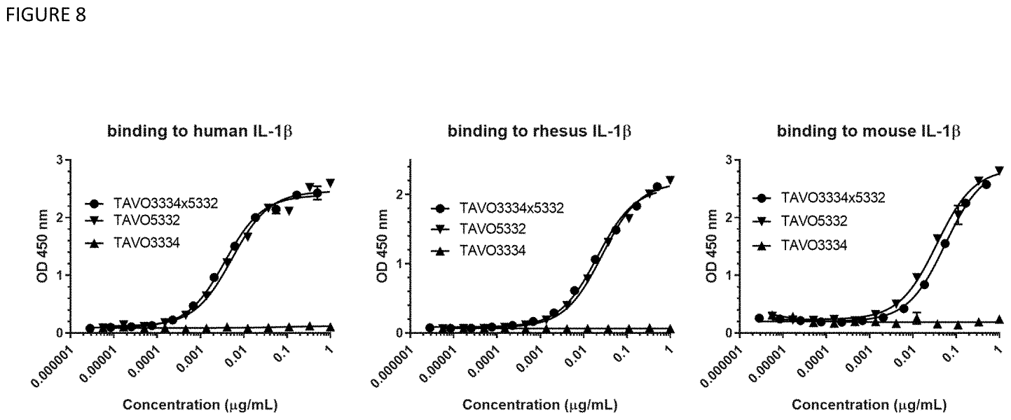

On the other hand, the anti-TNFα and IL-1β bispecific antibody TAVO3334×5332 also dose-dependently bound IL-1β from human, rhesus monkey and mouse with similar potency as that anti-IL-1β antibody TAVO5332, while anti-TNFα antibody TAVO3334 did not show binding activity (FIG. 8).

### Example 4: Neutralization TNFα Activity by Bispecific Antibody and their Respective Parental Antibodies

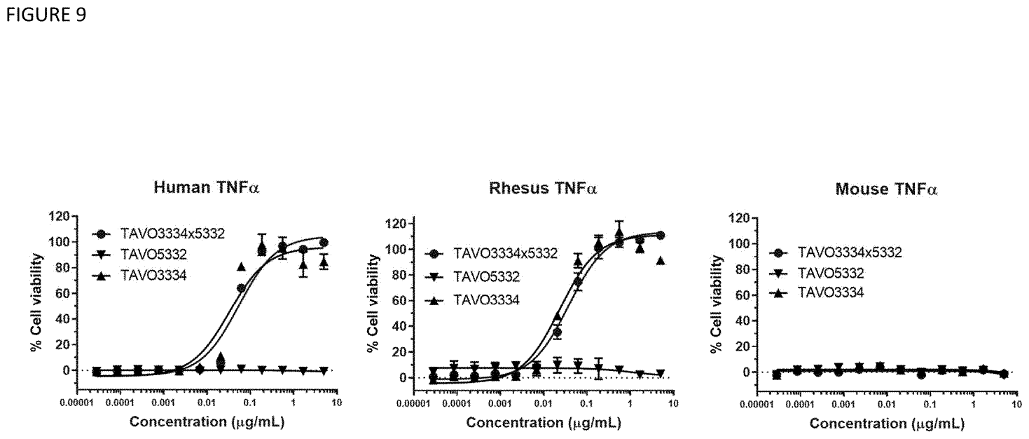

TNFα has cytotoxicity effect on a murine fibrosarcoma WEHI cell line. A WEHI cell-based cytotoxicity assay was developed to assess the effects of TAVO3334×5332 and its parental antibodies on the neutralization of TNFα-mediated cytotoxicity. In this assay, increasing amounts of testing antibodies were applied to WEHI cells along with 10 ng/mL TNFα. The cytotoxicity of WEHI cells was quantitated by MTT assay. It was observed that the anti-TNFα and IL-1β bispecific antibody TAVO3334×5332 dose-dependently neutralized cytotoxicity activity of TNFα from human and rhesus monkey with less than two-fold less potency relative to anti-TNFα antibody TAVO3334, while anti-IL1β antibody TAVO5332 did not show functional activity (FIG. 9). However, although with good binding affinity, neither the anti-TNFα and IL-113 bispecific antibody TAVO3334×5332 nor the anti-TNFα antibody TAVO3334 showed functional neutralization activity towards mouse TNFα.

### Example 5: Neutralization IL-1β Activity by Bispecific Antibody and their Respective Parental Antibodies

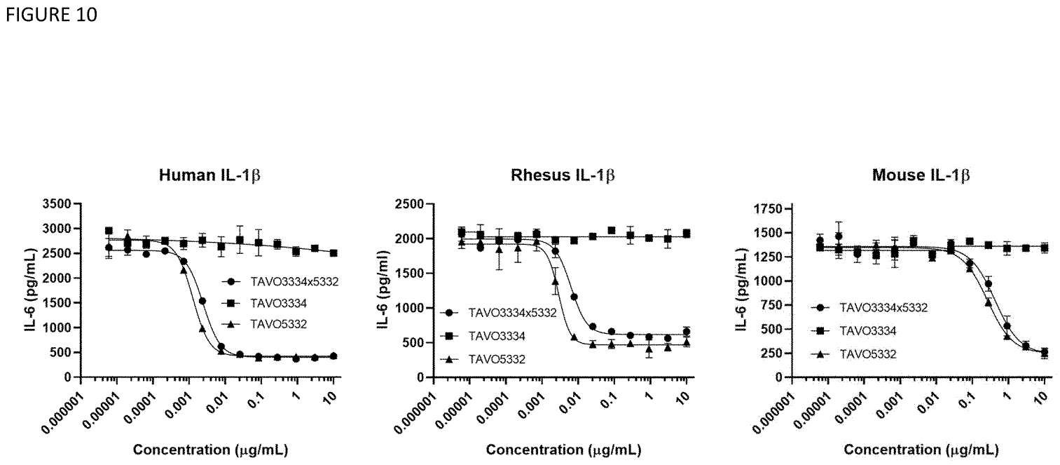

IL-1β can drive the activation of human lung fibroblast cell line MRC-5 and stimulate IL-6 release. A MRC-5 cell-based assay was employed to evaluate the effects of TAVO3334×5332 and its parental antibodies in blocking IL-6 release driven by IL-1β from human, rhesus monkey, and mouse respectively. Increasing amounts of antibodies along with IL-1β (1 ng/ml for human and rhesus monkey, and 10 ng/ml for mouse) were applied to 5,000 MRC-5 cells in each well of 96-well plate. After overnight incubation, the IL-6 production was quantitated by IL-6 assay kit (R&D systems). It was observed that the anti-TNFα and IL-1β bispecific antibody TAVO3334×5332 and its anti-IL-1β parental antibody TAVO5332 could dose-dependently inhibit IL-6 release induced by IL-1β from human, rhesus monkey, and mouse, while anti-TNFα antibody TAVO3334 did not show functional activity (FIG. 10). The anti-TNFα and IL-1β bispecific antibody TAVO3334×5332 showed slightly less potency (<2-fold) compared to its anti-IL-1β parental antibody TAVO5332 in neutralizing IL-1β activity.

### Example 6: Neutralization Both TNFα and IL-111 Activities by Bispecific Antibody and their Respective Parental Antibodies

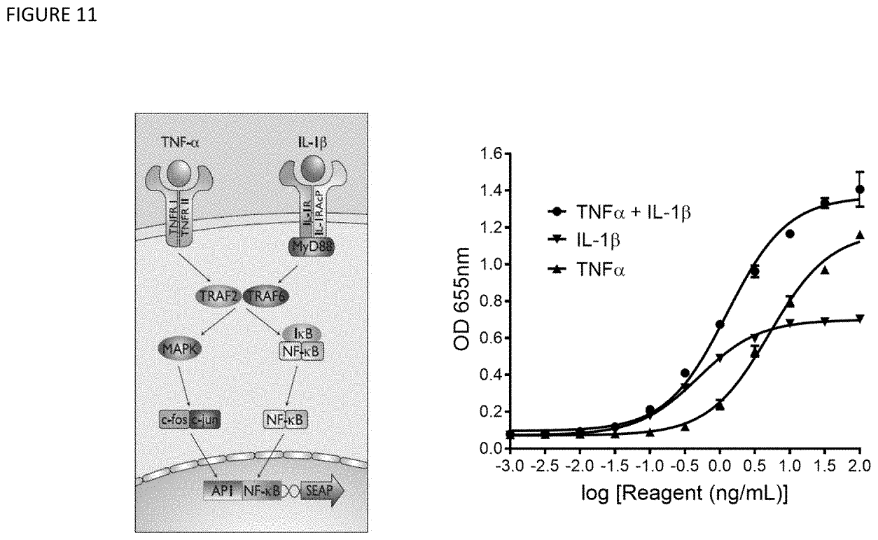

Functional activities of both TNFα and IL-1β can be assessed by a HEK-Blue reporter assay. In this assay, HEK-Blue null1-v cells (Invivogen) can respond to both TNFα and IL-1β stimulation by triggering a signalling cascade leading to the activation of NF-κB, and the subsequent production of a secreted embryonic alkaline phosphatase (SEAP) by activating the SEAP reporter gene expression (FIG. 11).

The response of HEK-Blue null1-v reporter cell line to TNFα and IL-1β was evaluated using this assay. It was observed that either TNFα or IL-1β could dose-dependently induce reporter gene expression with EC50 at 5 ng/mL and 0.5 ng/mL respectively (FIG. 11). The addition of both TNFα and IL-1β to the cells could elicit additive effects with higher activation of reporter gene expression and with EC50 at 1.25 ng/mL.

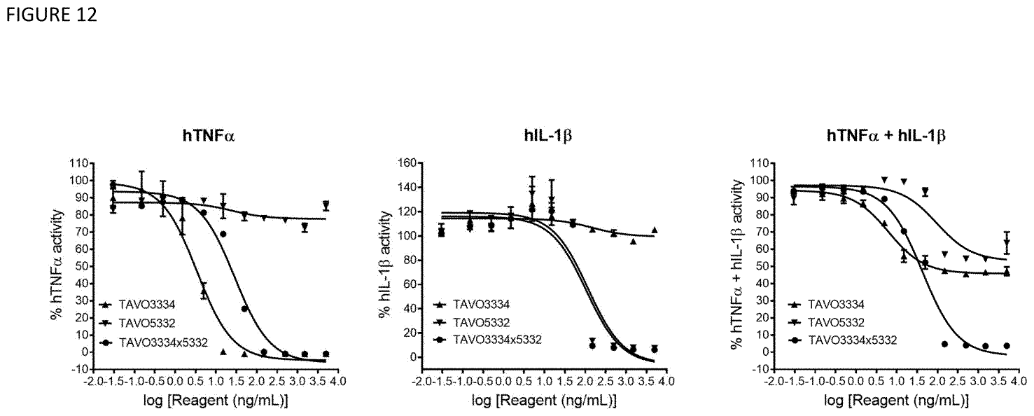

The HEK-Blue reporter assay was then employed to evaluate anti-TNFα and IL-1β bispecific antibody TAVO3334×5332 and its parent antibodies in blocking reporter gene expression driven by TNFα, IL-1β or TNFα and IL-1β together. Increasing amounts of antibodies along with TNFα and/or IL-1β were applied to HEK-Blue reporter cells. After overnight incubation, the SEAP reporter gene expression was quantitated. It was observed that the anti-TNFα and IL-1β bispecific antibody TAVO3334×5332 dose-dependently inhibited TNFα-mediated reporter gene activation similarly as that anti-TNFα antibody TAVO3334, while anti-IL1β antibody TAVO5332 did not show functional activity (FIG. 12). The anti-TNFα and IL-1β bispecific antibody TAVO3334×5332 also dose-dependently inhibited IL-1β-mediated reporter gene activation with similar potency as that anti-IL-1β antibody TAVO5332, while anti-TNFα antibody TAVO3334 did not show functional activity (FIG. 12).

The same assay was also employed to evaluate the bispecific antibody and its parental antibodies in blocking reporter gene activation driven by TNFα and IL-1β together. It was observed that both the anti-TNFα antibody TAVO3334 and the anti-IL-1β antibody TAVO5332 could dose-dependently inhibit reporter gene activation driven by TNFα and IL-1β together; however, they could only partially block reporter gene activation driven by both cytokines (FIG. 12). In contrast, the anti-TNFα and IL-1β bispecific antibody TAVO3334×5332 dose-dependently blocked reporter gene activation driven by TNFα and IL-1β together with full efficacy. This data demonstrated the functional activity of the bispecific antibody on both cytokines.

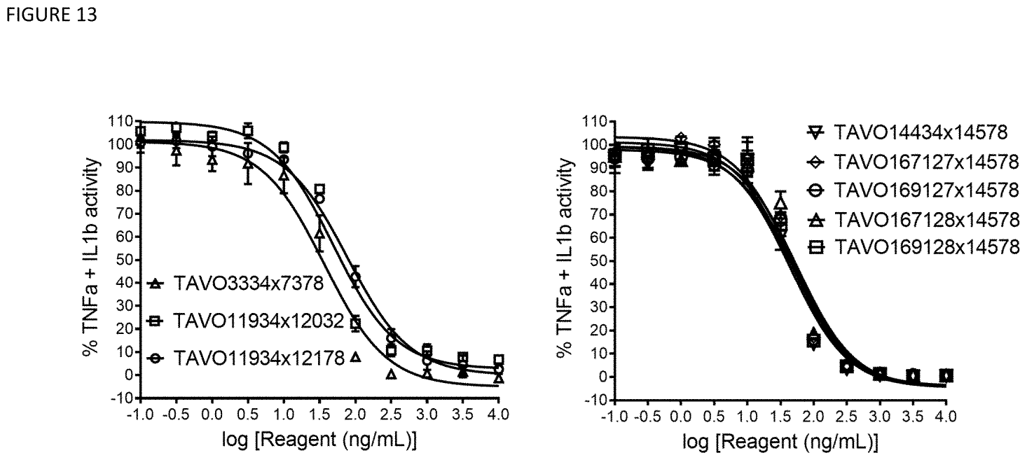

Besides TAVO3334×5332, the HEK-Blue reporter assay was also employed to evaluate other anti-TNFα and IL-1β bispecific antibodies in blocking reporter gene activation driven by TNFα and IL-1β together. It was observed that TAVO3334×7378, TAVO11934×12032, TAVO11934×12178, TAVO14434×14578, TAVO167127×14578, TAVO169127×14578, TAVO167128×14578, and TAVO169128×14578 all could dose-dependently inhibit reporter gene activation driven by TNFα and IL-1β together with full efficacy (FIG. 13).

### Example 7: F_{c} Engineering of Anti-TNFα and IL-1β Bispecific Antibodies for Extended Half-Life and Reduced Effector Functions

To improve the PK profile of anti-TNFα and IL-1β bispecific antibodies, F_{c} mutations can be introduced to IgG1 antibody to extend antibody half-life. Specifically, M428L/N434S mutations have been demonstrated to extend antibody half-life by increasing FcRn binding affinity (Booth, Ramakrishnan et al. 2018). Furthermore, L234A/L235A F_{c} mutations can abolish the ADCC and CDC effector functions of IgG1 antibody (Hezareh, Hessell et al. 2001). Therefore, two anti-TNFα and IL-1β bispecific antibodies, designated as TAVO11934×12032 and TAVO11934×12178, were generated with L234A, L235A, M428L, N434S (AALS) mutations in their IgG1 Fc.

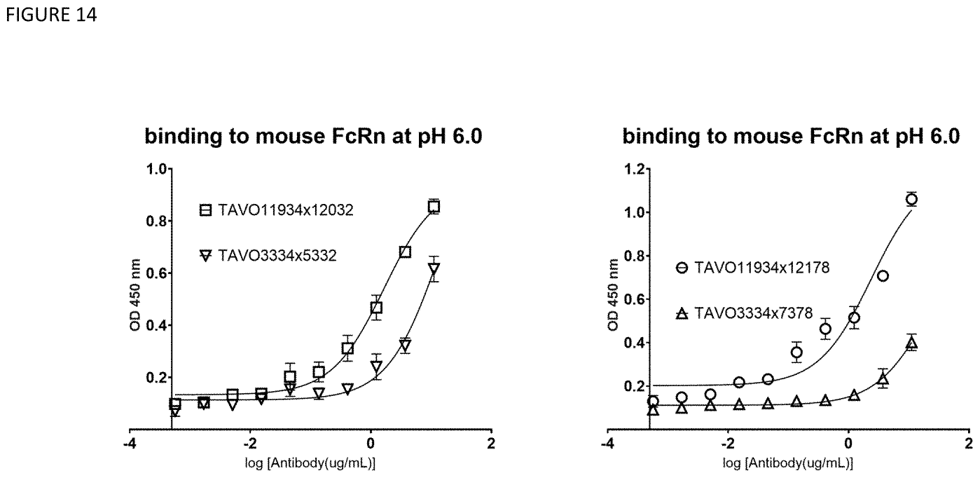

To study whether the Fc engineered antibody has improved FcRn binding affinity, the binding by TAVO11934×12032 and its counterpart antibody TAVO3334×5332 with wild-type IgG1 to mouse FcRn were assessed in ELISA-based binding assay. 1 μg/mL recombinant mouse FcRn (R&D systems) were coated on ELISA plate. Increasing concentrations of TAVO11934×12032 and TAVO3334×5332 antibodies were applied on the plate and their binding to the recombinant FcRn under pH 6.0 were detected by HRP-conjugated anti-human secondary antibody. It was observed that TAVO11934×12032, which has the M428L/N434S F_{c} mutations, could bind FcRn with better potency and efficacy than TAVO3334×5332 which is lacking such half-life extension mutations (FIG. 14). FcRn binding assay was also performed with another pair of anti-TNFα and IL-1β bispecific antibodies with or without half-life extension mutations. Similarly, it was observed that TAVO11934×12178, which has the F_{c} M428L/N434S mutations, could bind FcRn with better potency and efficacy than TAVO3334×7378 which is lacking such half-life extension mutations (FIG. 14).

To determine whether the M428L/N434S mutations could extend circulating half-life of an anti-TNFα and IL-1β bispecific antibody, TAVO11934×12032 will be tested in a cynomolgus monkey PK model. TAVO11934×12032 will be administered as an intravenously infusion at 4 mg/kg into a male naïve cynomolgus monkey at a volume of 1.0 ml/kg for 3 minutes based on the body weight on day 0. Whole blood will be collected into EDTA-K2 collection tubes at pre-dose, and at 1h, 2h, and on various times up to day 35 post-dose. Plasma will be separated by centrifugation at 3500×g for 10 minutes at 4° C., and then transferred to microfuge tubes for storage at −80° C. Plasma samples will be measured by a standard ELISA method to detect human IgG. PK data will be analyzed using Winnonlin 6.4 software. Based on published data and our previous study with another antibody with such M428L/N434S F_{c} mutations, which had a half-life around 26 days, it is predicted that TAVO11934×12032 will have a much longer circulating half-life in monkey than a normal human IgG.

### Example 8: F_{c} Engineering of Anti-TNFα and IL-1β Bispecific Antibodies for Resistance to Protease Degradation

To improve the in vivo stability of anti-TNFα and IL-1β bispecific antibodies, F_{c} mutations can be introduced to IgG1 antibody to enhance the antibody resistant to proteolytic degradation. Many proteases may cleave the wild-type IgG antibody between or at residues 222-237 (EU numbering). The resistance to proteolytic degradation can be realized by engineering E233P, L234A, L235A mutations in the hinge region of IgG1 antibody with G236 deleted, residue numbering according to the EU Index (Kinder, Greenplate et al. 2013). To endow anti-TNFα and IL1β bispecific antibodies with optimal properties, a series of Fc mutations, including E233P, L234A, L235A, F405L, M428L, N434S mutations with G236 deleted, were introduced to a number of anti-TNFα and IL-1β bispecific antibodies listed in Table 4. This set of mutations include F_{c} mutations to enhance the antibody resistant to proteolytic degradation, along with M428L/N434S mutations to extend antibody half-life and L234A/L235A mutations to abolish ADCC and CDC effector functions.

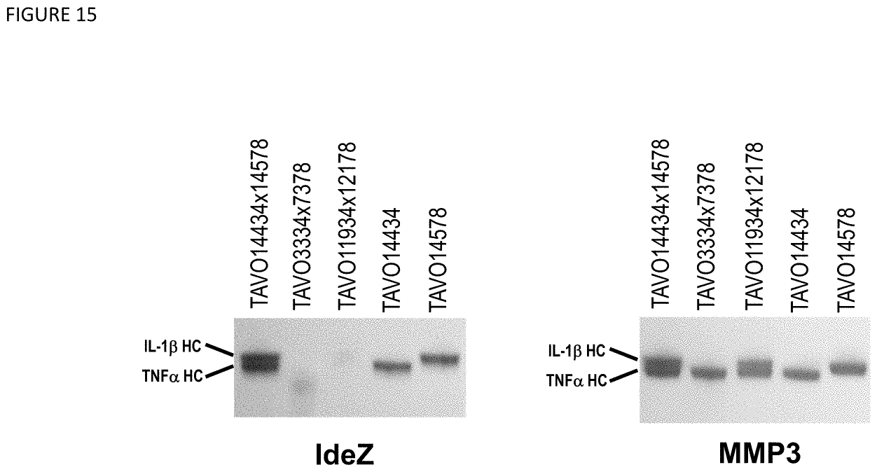

To study whether the anti-TNFα and IL-1β bispecific antibodies engineered with these F_{c} mutations has improved resistance to proteolytic degradation, a set of antibodies with different IgG1 F_{c} mutations were subjected to digestion by recombinant IgG protease IdeZ (New England Biolabs) at 37° C. for half an hour followed by SDS-PAGE analysis under reduced condition to assess the integrity of heavy chains. It was observed that TAVO14434×14578, an anti-TNFα and IL-1β bispecific IgG1 antibody engineered with E233P, L234A, L235A, F405L, M428L, N434S Fc mutations and with G236 deleted, has intact anti-TNFα heavy chain band and anti-IL-1β heavy chain band which have close migration on the gel (FIG. 15). Similarly, its parental antibodies with the same set of Fc mutations, TAVO14434 and TAVO14578, were also resistant to proteolytic degradation by IdeZ. However, neither anti-TNFα and IL-1β bispecific antibody TAVO3334×7378, which has no mutations in its IgG1 Fc, nor TAVO11934×12178, which has L234A, L235A, M428L, N434S (AALS) mutations in its IgG1 Fc, could resist digestion by IdeZ and both anti-TNFα heavy chain band and anti-IL-1β heavy chain band were missing (FIG. 15). This indicated the E233P, L234A, L235A F_{c} mutations with G236 deleted could facilitate anti-TNFα and IL-1β bispecific antibodies resistant to IdeZ degradation.

Besides IgG protease IdeZ, the same set of antibodies with different IgG1 F_{c} mutations were also subjected to digestion by recombinant Matrix Metalloproteinase 3, MMP3 (Enzo Life Sciences) at 37° C. for 24 hours followed by SDS-PAGE under reduced condition to assess the integrity of heavy chains. It was observed that the anti-TNFα heavy chain remained intact upon MMP3 digestion, no matter whether its IgG1 F_{c} has proteolytic resistant mutations or not (FIG. 15). However, the anti-IL-1β heavy chain band was missing in TAVO3334×7378 which has no mutations in its IgG1 Fc, but remained intact in TAVO14434×14578, which is an anti-TNFα and IL-1β bispecific IgG1 antibody engineered with E233P, L234A, L235A, F405L, M428L, N434S Fc mutations and with G236 deleted, and TAVO11934×12178, which has L234A, L235A, M428L, N434S (AALS) mutations in its IgG1 Fc (FIG. 15). This indicated that Fc mutations below the hinge region are needed to facilitate anti-TNFα and IL-1β bispecific antibodies resistant to MMP3 degradation.

Whether these extensive F_{c} mutations could affect the functional activities of anti-TNFα and IL-1β bispecific antibodies were evaluated in HEK-Blue reporter assay. As shown in FIG. 13, TAVO14434×14578, TAVO167127×14578, TAVO169127×14578, TAVO167128×14578, and TAVO169128×14578, which are all engineered with E233P, L234A, L235A, F405L, M428L, N434S Fc mutations and with G236 deleted, could dose-dependently inhibit reporter gene activation driven by TNFα and IL-1β together with full efficacy and similar potency as corresponding anti-TNFα and IL-1β bispecific antibodies without such mutations.

### Example 9: In Vivo Efficacy of Anti-TNFα and IL-1β Bispecific Antibody in a Model of Collagen Antibody Induced Arthritis

The efficacy of anti-TNFα and IL-1β bispecific antibody TAVO3334×5332 in inflammation was evaluated in a collagen antibody induced arthritis (CAIA) model (Moore, Allden et. Al, 2014). CAIA model was established through the administration of an anti-collagen monoclonal antibody cocktail and the subsequent administration of lipopolysaccharide (LPS). CAIA is characterized by inflammation, pannus formation and bone erosions similar to those observed in RA. The CAIA pathology has been reported to be TNFα and IL-1β dependent, while blockade with anti-TNFα or anti-IL1β antibody has been shown to ameliorate the pathology (Bendele, Chlipala et al, 2000).

Since anti-TNFα and IL-1β bispecific antibody TAVO3334×5332 cannot neutralize mouse TNFα activity even though there was good binding affinity to mouse TNFα, the study was conducted using Tg1278/TNFKO mice provided by Biomedcode, Greece. Tg1278/TNFKO mice lack murine TNFα and are heterozygous for multiple copies of the human TNFα transgene that is expressed under normal physiological control. Tg1278/TNFKO mice exhibit normal development with no overt pathology. CAIA was induced in 8 to 10-week-old Tg1278/TNFKO male mice that received intraperitoneal injections (i.p.) of arthritogenic antibody cocktail (ArthritoMab, MD Biosciences) on day 0, followed by an i.p. injection of LPS on Day 3. After CAIA induction, PBS or 3 dose concentrations of TAVO3334×5332 (1 mg/kg, 5 mg/kg and 10 mg/kg) were dosed twice per week for two weeks. The clinical scores of arthritis, histopathology of the limbs and body weight were measured and collected as the read out.

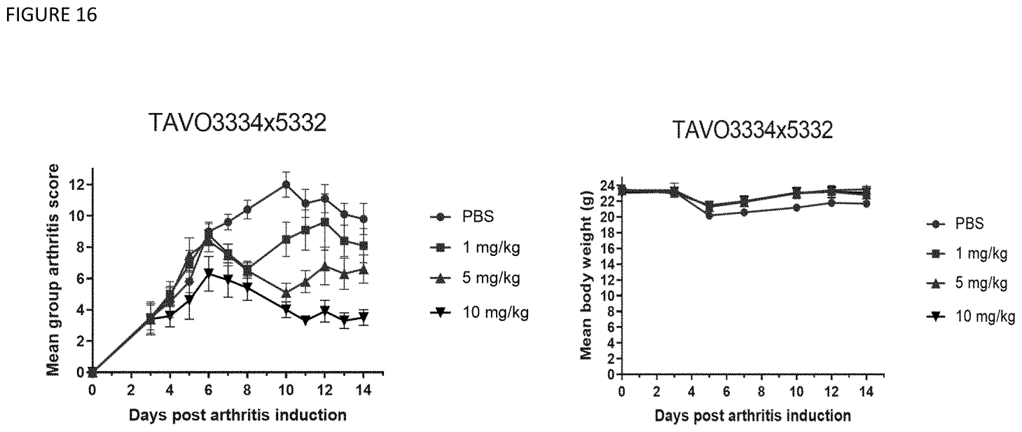

Results of the study showed that by day 14 post induction, the PBS treated group displayed dramatically increased in vivo arthritic scores demonstrating induction of the arthritic pathology. Treatment with TAVO3334×5332 at 1 mg/kg, 5 mg/kg and 10 mg/kg inhibited the arthritic phenotype in a dose-dependent manner compared to the negative control PBS treated group (FIG. 16, left panel). By Day 14 post-dose, the 10 mg/kg, 5 mg/kg and 1 mg/kg doses inhibited arthritic scores by 65%, 32% and 17%, respectively, compared to the PBS arthritic score. Besides, Mice dosed with TAVO3334×5332 showed minimal weight loss in contrast to mice treated with PBS which showed significant 7% weight loss over 14 days (FIG. 16, right panel). Overall, results of the study provided evidence of the therapeutic effect of TAVO3334×5332 in preventing arthritic symptoms in a CAIA model induced in the Tg1278/TNFKO mice.

### Example 10: In Vivo Efficacy of Anti-TNFα and IL-1β Bispecific Antibody in a Model of Knee Joint Inflammation

A mouse model of knee joint inflammation was also developed to evaluate the in vivo efficacy of anti-TNFα and IL-1β bispecific antibody TAVO11934×12178 in normal mice. The joint inflammation in this model was induced upon continuous secretion of human TNFα and IL-1β from transfected mouse NIH3T3 cells injected into one of the knee joint, since both human TNFα and IL-1β can activate cognate receptors in mice to induce inflammation. This model allows the study of anti-TNFα and IL-1β antibodies which can neutralize the effects of human cytokines but lacking the cross-reactivity to murine cytokines.

For the development of this model, murine fibroblast cell line NIH3T3, derived from a DBA-1 mouse background, was transfected with constructs expressing either human TNFα or IL-1β and two NIH3T3 cell lines stably expressing either of these two cytokines were thus established. The amount of human TNFα and IL-1β secreted from the established stable cell lines were quantitated by ELISA kits (Biolegend). It was observed that one million NIH3T3: hTNFα cells could secrete 10-30 ng hTNFα during 24 hour period, while the established NIH3T3: hIL1β cells could secrete 5-10 ng hIL-1β. Besides, both TNFα and IL-1β secreted from the stable NIH3T3 cell lines could activate reporter gene expression in HEK-Blue reporter assays for these cytokines (Invivogen), confirming functional activities for both secreted cytokines.

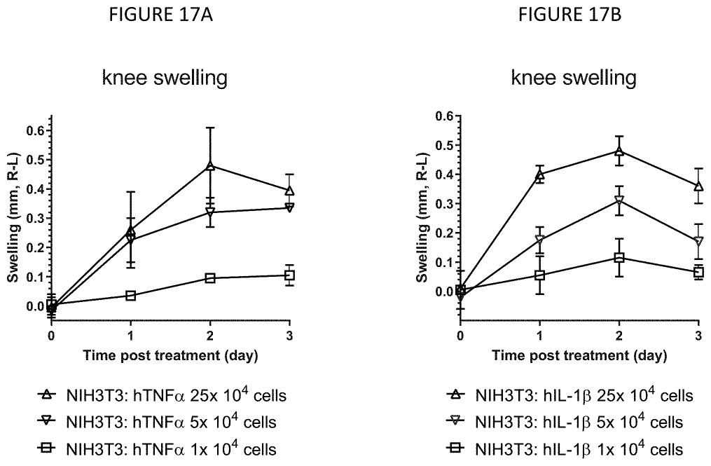

To assess the utility of the established cell lines in inducing knee joint inflammation, 1×10⁴, 5×10⁴, or 25×10⁴ of NIH3T3: hTNFα cells or NIH3T3: hIL-1β cells were injected into the right knee of male DBA-1 mice of 9-10 weeks old, while the left knee was injected with equivalent numbers of NIH3T3 parental cells. Caliper measurements of both knee joints were conducted each day after cell injection for three days and cytokine induced knee joint inflammation was quantitated as the caliper measurement difference between the treated right knee and untreated left knee. It was observed that both hTNFα and hIL-1β secreted from the injected cells could induce increased knee inflammation over the course of three days after cell injection in a cell number dependent manner (FIG. 17).

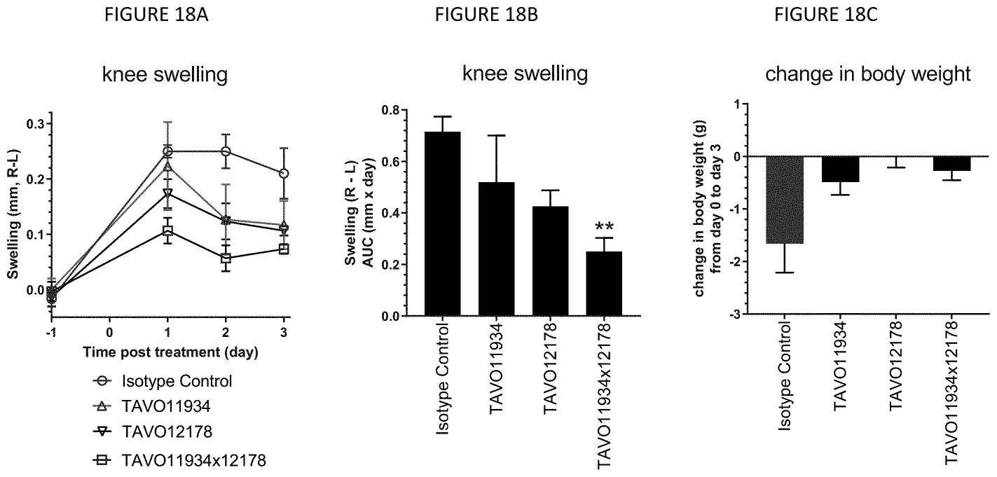

To study the in vivo efficacy of anti-TNFα and IL-1β bispecific antibody TAVO11934×12178 and its associated parental antibodies, these test articles along with isotype control antibody were dosed intraperitoneally into the DBA-1 mice two hours prior the mice were given an intra-articular (IA) injection of a mixture of 5×10⁴ NIH3T3: hTNFα cells and 5×10⁴ NIH3T3: hIL-1β cells into the right knee joint and 10×10⁴ NIH3T3 parental cells into the left knee as a control. Caliper measurements on both knees were taken on Day −1, and Days 1, 2 and 3 post injection and knee joint inflammation was quantitated as the caliper measurement difference between the treated right knee and untreated left knee. It was observed that TAVO11934×12178, dosed at 10 mg/kg, significantly suppressed knee joint inflammation induced by human TNFα and IL-1β compared to isotype control group (FIG. 18A). By Area Under the Curve (AUC) calculation, swelling in the TAVO11934×12178 bispecific antibody treated knees was reduced significantly (p value <0.005) by Day 3 (with AUC=0.25±0.05 mm×day) compared to the isotype control antibody treated knees (with AUC=0.72±0.06 mm×day) (FIG. 18B). However, both the parental anti-TNFα antibody TAVO11934 and the parental anti-IL-1β antibody TAVO12178, when dosed at 5 mg/kg which is equivalent in molarity to the bispecific antibody, could not induce the same degree suppression of knee joint inflammation (with AUC=0.52±0.18 mm×day for TAVO11934 and 0.43±0.06 mm×day for TAVO12178) as the bispecific antibody TAVO11934×12178, although the suppressions were still significant relative to the isotype control treated mice (FIG. 18A, 18B). Besides knee joint inflammation, it was observed that mice dosed with anti-TNFα and anti-IL-1β antibodies had minimal weight loss while mice treated with isotype control antibody showed more significant weight loss (FIG. 18C). These results demonstrated that the anti-TNFα and IL-1β bispecific antibody TAVO11934×12178 could neutralize the biological activity of both human TNFα and human IL-1β in inducing knee joint inflammation, while either anti-TNFα antibody or anti-IL-1β antibody alone could only show partial efficacy just by blocking only one of the two cytokines.

### SEQUENCES

Provided herein is a representative list of certain sequences included in embodiments provided herein.

TABLE 5
Sequences

| SEQ | | |
|---|---|---|
| ID | | |
| NO | Description | Sequence |
| 1 | Heavy chain variable | EVQLVESGGGLVQPGRSLRLSCAASGFTFDDYAMHWVRQAP |
| | domain ADA-H of | GKGLEWVSAITWNSGHIDYADSVEGRFTISRDNAKNSLYLQ |
| | anti-TNFα antibody | MNSLRAEDTAVYYCAKVSYLSTASSLDYWGQGTLVTVSS |
| 2 | Heavy chain variable | EVQLVESGGVVVQPGGSLRLSCAASGFTFDDYAMHWVRQAP |
| | domain ADA-H1 of | GKGLEWVSAITWNSGHIDYADSVKGRFTISRDNSKNSLYLQ |
| | anti-TNFα antibody | MNSLRTEDTALYYCAKVSYLSTASSLDYWGQGTLVTVSS |
| 3 | Heavy chain variable | EVQLVESGGVVVQPGGSLRLSCAASGFDFADYAMHWVRQAP |
| | domain ADA-H1X of | GKGLEWVSAITWNGGHTDYADSVKGRFTISRDNSKNSLYLQ |
| | anti-TNFα antibody | MNSLRTEDTALYYCAKVSYLSTASSLDYWGQGTLVTVSS |
| 4 | Heavy chain variable | EVQLVESGGGLVQPGGSLRLSCAASGFTFDDYAMHWVRQAP |
| | domain ADA-H2 of | GKGLEWVSAITWNSGHIDYADSVKGRFTISRDNSKNTLYLQ |
| | anti-TNFα antibody | MNSLRAEDTAVYYCAKVSYLSTASSLDYWGQGTLVTVSS |
| 5 | Heavy chain variable | EVQLVESGGGLVQPGGSLRLSCAASGFDFADYAMHWVRQAP |
| | domain ADA-H2X of | GKGLEWVSAITWNGGHTDYADSVKGRFTISRDNSKNTLYLQ |
| | anti-TNFα antibody | MNSLRAEDTAVYYCAKVSYLSTASSLDYWGQGTLVTVSS |
| 6 | Heavy chain variable | EVQLVESGGGLVQPGGSLRLSCAASGFTFDDYAMHWVRQAP |
| | domain ADA-H3 of | GKGLVWVSAITWNSGHIDYADSVKGRFTISRDNAKNTLYLQ |
| | anti-TNFα antibody | MNSLRAEDTAVYYCAKVSYLSTASSLDYWGQGTLVTVSS |
| 7 | Heavy chain variable | EVQLVESGGGLVQPGGSLRLSCAASGFDFADYAMHWVRQAP |
| | domain ADA-H3X of | GKGLVWVSAITWNGGHTDYADSVKGRFTISRDNAKNTLYLQ |
| | anti-TNFα antibody | MNSLRAEDTAVYYCAKVSYLSTASSLDYWGQGTLVTVSS |
| 8 | Heavy chain variable | QVQLVESGGGVVQPGGSLRLSCAASGFTFDDYAMHWVRQAP |
| | domain ADA-H4 of | GKGLEWVSAITWNSGHIDYADSVKGRFTISRDNSKNTLYLQ |
| | anti-TNFα antibody | MNSLRAEDTAVYYCAKVSYLSTASSLDYWGQGTLVTVSS |
| 9 | Heavy chain variable | QVQLVESGGGVVQPGGSLRLSCAASGFDFADYAMHWVRQAP |
| | domain ADA-H4X of | GKGLEWVSAITWNGGHTDYADSVKGRFTISRDNSKNTLYLQ |
| | anti-TNFα antibody | MNSLRAEDTAVYYCAKVSYLSTASSLDYWGQGTLVTVSS |
| 10 | Light chain variable | DIQMTQSPSSLSASVGDRVTITCRASQGIRNYLAWYQQKPG |
| | domain ADA-L of | KAPKLLIYAASTLQSGVPSRFSGSGSGTDFTLTISSLQPED |
| | anti-TNFα antibody | VATYYCQRYNRAPYTFGQGTKVEIK |
| 11 | Light chain variable | EIVMTQSPATLSVSPGERATLSCRASQGIRNYLAWYQQKPG |
| | domain ADA-L1 of | QAPRLLIYAASTLQSGIPARFSGSGSGTEFTLTISSLQSED |
| | anti-TNFα antibody | FAVYYCQRYNRAPYTFGQGTKVEIK |
| 12 | Light chain variable | DIVMTQSPDSLAVSLGERATINCRASQGIRNYLAWYQQKPG |
| | domain ADA-L2 of | QAPKLLIYAASTLQSGVPDRFSGSGSGTDFTLTISSLQAED |
| | anti-TNFα antibody | VAVYYCQRYNRAPYTFGQGTKVEIK |
| 13 | anti-TNFα antibody | EVQLVESGGGLVQPGRSLRLSCAASGFTFDDYAMHWVRQAP |
| | heavy chain EAC33 | GKGLEWVSAITWNSGHIDYADSVEGRFTISRDNAKNSLYLQ |
| | based on ADA-H | MNSLRAEDTAVYYCAKVSYLSTASSLDYWGQGTLVTVSSAS |
| | variable domain | TKGPSVFPLAPSSKSTSGGTAALGCLVKDYFPEPVTVSWNS |
| | (underlined) with | GALTSGVHTFPAVLQSSGLYSLSSVVTVPSSSLGTQTYICN |
| | IgG1 Fc with F405L | VNHKPSNTKVDKKVEPKSCDKTHTCPPCPAPELLGGPSVFL |
| | mutation (bolded) | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSREEMTKNQVSL |
| | | TCLVKGFYPSDIAVEWESNGQPENNYKTTPPVLDSDGSFLL |
| | | YSKLTVDKSRWQQGNVFSCSVMHEALHNHYTQKSLSLSPGK |
| 14 | anti-TNFα antibody | EVQLVESGGGLVQPGRSLRLSCAASGFTFDDYAMHWVRQAP |
| | heavy chain EAC119 | GKGLEWVSAITWNSGHIDYADSVEGRFTISRDNAKNSLYLQ |
| | based on ADA-H | MNSLRAEDTAVYYCAKVSYLSTASSLDYWGQGTLVTVSSAS |
| | variable domain | TKGPSVFPLAPSSKSTSGGTAALGCLVKDYFPEPVTVSWNS |
| | (underlined) with | GALTSGVHTFPAVLQSSGLYSLSSVVTVPSSSLGTQTYICN |
| | IgG1 Fc with L234A, | VNHKPSNTKVDKKVEPKSCDKTHTCPPCPAPEAAGGPSVFL |
| | L235A, F405L, | FPPKPKDTLMISRTPEVTCVVVDVSHEDPEVKFNWYVDGVE |
| | M428L, N434S | VHNAKTKPREEQYNSTYRVVSVLTVLHQDWLNGKEYKCKVS |
| | mutations (bolded) | NKALPAPIEKTISKAKGQPREPQVYTLPPSRDELTKNQVSL |
| | | TCLVKGFYPSDIAVEWESNGQPENNYKTTPPVLDSDGSFLL |
| | | YSkLTVDKSRWQQGNVFSCSVLHEALHSHYTQKSLSLSPGK |
| 15 | anti-TNFα antibody | EVQLVESGGVVVQPGGSLRLSCAASGFTFDDYAMHWVRQAP |
| | heavy chain EAC129 | GKGLEWVSAITWNSGHIDYADSVKGRFTISRDNSKNSLYLQ |
| | based on ADA-H1 | MNSLRTEDTALYYCAKVSYLSTASSLDYWGQGTLVTVSSAS |
| | variable domain | TKGPSVFPLAPSSKSTSGGTAALGCLVKDYFPEPVTVSWNS |
| | (underlined) with | GALTSGVHTFPAVLQSSGLYSLSSVVTVPSSSLGTQTYICN |
| | IgG1 Fc with L234A, | VNHKPSNTKVDKKVEPKSCDKTHTCPPCPAPEAAGGPSVFL |
| | L235A, F405L, | FPPKPKDTLMISRTPEVTCVVVDVSHEDPEVKFNWYVDGVE |
| | M428L, N434S | VHNAKTKPREEQYNSTYRVVSVLTVLHQDWLNGKEYKCKVS |
| | mutations (bolded) | NKALPAPIEKTISKAKGQPREPQVYTLPPSRDELTKNQVSL |
| | | TCLVKGFYPSDIAVEWESNGQPENNYKTTPPVLDSDGSFLL |
| | | YSkLTVDKSRWQQGNVFSCSVLHEALHSHYTQKSLSLSPGK |
| 16 | anti-TNFα antibody | EVQLVESGGVVVQPGGSLRLSCAASGFDFADYAMHWVRQAP |
| | heavy chain EAC130 | GKGLEWVSAITWNGGHTDYADSVKGRFTISRDNSKNSLYLQ |
| | based on ADA-H1X | MNSLRTEDTALYYCAKVSYLSTASSLDYWGQGTLVTVSSAS |
| | variable domain | TKGPSVFPLAPSSKSTSGGTAALGCLVKDYFPEPVTVSWNS |
| | (underlined) with | GALTSGVHTFPAVLQSSGLYSLSSVVTVPSSSLGTQTYICN |
| | IgG1 Fc with L234A, | VNHKPSNTKVDKKVEPKSCDKTHTCPPCPAPEAAGGPSVFL |
| | L235A, F405L, | FPPKPKDTLMISRTPEVTCVVVDVSHEDPEVKFNWYVDGVE |
| | M428L, N434S | VHNAKTKPREEQYNSTYRVVSVLTVLHQDWLNGKEYKCKVS |
| | mutations (bolded) | NKALPAPIEKTISKAKGQPREPQVYTLPPSRDELTKNQVSL |
| | | TCLVKGFYPSDIAVEWESNGQPENNYKTTPPVLDSDGSFLL |
| | | YSkLTVDKSRWQQGNVFSCSVLHEALHSHYTQKSLSLSPGK |
| 17 | anti-TNFα antibody | EVQLVESGGGLVQPGGSLRLSCAASGFTFDDYAMHWVRQAP |
| | heavy chain EAC131 | GKGLEWVSAITWNSGHIDYADSVKGRFTISRDNSKNTLYLQ |
| | based on ADA-H2 | MNSLRAEDTAVYYCAKVSYLSTASSLDYWGQGTLVTVSSAS |
| | variable domain | TKGPSVFPLAPSSKSTSGGTAALGCLVKDYFPEPVTVSWNS |
| | (underlined) with | GALTSGVHTFPAVLQSSGLYSLSSVVTVPSSSLGTQTYICN |
| | IgG1 Fc with L234A, | VNHKPSNTKVDKKVEPKSCDKTHTCPPCPAPEAAGGPSVFL |
| | L235A, F405L, | FPPKPKDTLMISRTPEVTCVVVDVSHEDPEVKFNWYVDGVE |
| | M428L, N434S | VHNAKTKPREEQYNSTYRVVSVLTVLHQDWLNGKEYKCKVS |
| | mutations (bolded) | NKALPAPIEKTISKAKGQPREPQVYTLPPSRDELTKNQVSL |
| | | TCLVKGFYPSDIAVEWESNGQPENNYKTTPPVLDSDGSFLL |
| | | YSkLTVDKSRWQQGNVFSCSVLHEALHSHYTQKSLSLSPGK |
| 18 | anti-TNFα antibody | EVQLVESGGGLVQPGGSLRLSCAASGFDFADYAMHWVRQAP |
| | heavy chain EAC132 | GKGLEWVSAITWNGGHTDYADSVKGRFTISRDNSKNTLYLQ |
| | based on ADA-H2X | MNSLRAEDTAVYYCAKVSYLSTASSLDYWGQGTLVTVSSAS |
| | variable domain | TKGPSVFPLAPSSKSTSGGTAALGCLVKDYFPEPVTVSWNS |
| | (underlined) with | GALTSGVHTFPAVLQSSGLYSLSSVVTVPSSSLGTQTYICN |
| | IgG1 Fc with L234A, | VNHKPSNTKVDKKVEPKSCDKTHTCPPCPAPEAAGGPSVFL |
| | L235A, F405L, | FPPKPKDTLMISRTPEVTCVVVDVSHEDPEVKFNWYVDGVE |
| | M428L, N434S | VHNAKTKPREEQYNSTYRVVSVLTVLHQDWLNGKEYKCKVS |
| | mutations (bolded) | NKALPAPIEKTISKAKGQPREPQVYTLPPSRDELTKNQVSL |
| | | TCLVKGFYPSDIAVEWESNGQPENNYKTTPPVLDSDGSFLL |
| | | YSkLTVDKSRWQQGNVFSCSVLHEALHSHYTQKSLSLSPGK |
| 19 | anti-TNFα antibody | EVQLVESGGGLVQPGGSLRLSCAASGFTFDDYAMHWVRQAP |
| | heavy chain EAC133 | GKGLVWVSAITWNSGHIDYADSVKGRFTISRDNAKNTLYLQ |
| | based on ADA-H3 | MNSLRAEDTAVYYCAKVSYLSTASSLDYWGQGTLVTVSSAS |
| | variable domain | TKGPSVFPLAPSSKSTSGGTAALGCLVKDYFPEPVTVSWNS |
| | (underlined) with | GALTSGVHTFPAVLQSSGLYSLSSVVTVPSSSLGTQTYICN |
| | IgG1 Fc with L234A, | VNHKPSNTKVDKKVEPKSCDKTHTCPPCPAPEAAGGPSVFL |
| | L235A, F405L, | FPPKPKDTLMISRTPEVTCVVVDVSHEDPEVKFNWYVDGVE |
| | M428L, N434S | VHNAKTKPREEQYNSTYRVVSVLTVLHQDWLNGKEYKCKVS |
| | mutations (bolded) | NKALPAPIEKTISKAKGQPREPQVYTLPPSRDELTKNQVSL |
| | | TCLVKGFYPSDIAVEWESNGQPENNYKTTPPVLDSDGSFLL |
| | | YSkLTVDKSRWQQGNVFSCSVLHEALHSHYTQKSLSLSPGK |
| 20 | anti-TNFα antibody | EVQLVESGGGLVQPGGSLRLSCAASGFDFADYAMHWVRQAP |
| | heavy chain EAC134 | GKGLVWVSAITWNGGHTDYADSVKGRFTISRDNAKNTLYLQ |
| | based on ADA-H3X | MNSLRAEDTAVYYCAKVSYLSTASSLDYWGQGTLVTVSSAS |
| | variable domain | TKGPSVFPLAPSSKSTSGGTAALGCLVKDYFPEPVTVSWNS |
| | (underlined) with | GALTSGVHTFPAVLQSSGLYSLSSVVTVPSSSLGTQTYICN |
| | IgG1 Fc with L234A, | VNHKPSNTKVDKKVEPKSCDKTHTCPPCPAPEAAGGPSVFL |
| | L235A, F405L, | FPPKPKDTLMISRTPEVTCVVVDVSHEDPEVKFNWYVDGVE |
| | M428L, N434S | VHNAKTKPREEQYNSTYRVVSVLTVLHQDWLNGKEYKCKVS |
| | mutations (bolded) | NKALPAPIEKTISKAKGQPREPQVYTLPPSRDELTKNQVSL |
| | | TCLVKGFYPSDIAVEWESNGQPENNYKTTPPVLDSDGSFLL |
| | | YSkLTVDKSRWQQGNVFSCSVLHEALHSHYTQKSLSLSPGK |
| 21 | anti-TNFα antibody | QVQLVESGGGVVQPGGSLRLSCAASGFTFDDYAMHWVRQAP |
| | heavy chain EAC135 | GKGLEWVSAITWNSGHIDYADSVKGRFTISRDNSKNTLYLQ |
| | based on ADA-H4 | MNSLRAEDTAVYYCAKVSYLSTASSLDYWGQGTLVTVSSAS |
| | variable domain | TKGPSVFPLAPSSKSTSGGTAALGCLVKDYFPEPVTVSWNS |
| | (underlined) with | GALTSGVHTFPAVLQSSGLYSLSSVVTVPSSSLGTQTYICN |
| | IgG1 Fc with L234A, | VNHKPSNTKVDKKVEPKSCDKTHTCPPCPAPEAAGGPSVFL |
| | L235A, F405L, | FPPKPKDTLMISRTPEVTCVVVDVSHEDPEVKFNWYVDGVE |
| | M428L, N434S | VHNAKTKPREEQYNSTYRVVSVLTVLHQDWLNGKEYKCKVS |
| | mutations (bolded) | NKALPAPIEKTISKAKGQPREPQVYTLPPSRDELTKNQVSL |
| | | TCLVKGFYPSDIAVEWESNGQPENNYKTTPPVLDSDGSFLL |
| | | YSkLTVDKSRWQQGNVFSCSVLHEALHSHYTQKSLSLSPGK |
| 22 | anti-TNFα antibody | QVQLVESGGGVVQPGGSLRLSCAASGFDFADYAMHWVRQAP |
| | heavy chain EAC136 | GKGLEWVSAITWNGGHTDYADSVKGRFTISRDNSKNTLYLQ |
| | based on ADA-H4X | MNSLRAEDTAVYYCAKVSYLSTASSLDYWGQGTLVTVSSAS |
| | variable domain | TKGPSVFPLAPSSKSTSGGTAALGCLVKDYFPEPVTVSWNS |
| | (underlined) with | GALTSGVHTFPAVLQSSGLYSLSSVVTVPSSSLGTQTYICN |
| | IgG1 Fc with L234A, | VNHKPSNTKVDKKVEPKSCDKTHTCPPCPAPEAAGGPSVFL |
| | L235A, F405L, | FPPKPKDTLMISRTPEVTCVVVDVSHEDPEVKFNWYVDGVE |
| | M428L, N434S | VHNAKTKPREEQYNSTYRVVSVLTVLHQDWLNGKEYKCKVS |
| | mutations (bolded) | NKALPAPIEKTISKAKGQPREPQVYTLPPSRDELTKNQVSL |
| | | TCLVKGFYPSDIAVEWESNGQPENNYKTTPPVLDSDGSFLL |
| | | YSkLTVDKSRWQQGNVFSCSVLHEALHSHYTQKSLSLSPGK |
| 23 | anti-TNFα antibody | EVQLVESGGGLVQPGRSLRLSCAASGFTFDDYAMHWVRQAP |
| | heavy chain EAC144 | GKGLEWVSAITWNSGHIDYADSVEGRFTISRDNAKNSLYLQ |
| | based on ADA-H | MNSLRAEDTAVYYCAKVSYLSTASSLDYWGQGTLVTVSSAS |
| | variable domain | TKGPSVFPLAPSSKSTSGGTAALGCLVKDYFPEPVTVSWNS |
| | (underlined) with | GALTSGVHTFPAVLQSSGLYSLSSVVTVPSSSLGTQTYICN |
| | IgG1 Fc with E233P | VNHKPSNTKVDKKVEPKSCDKTHTCPPCPAPPAAGPSVFLF |
| | L234A, L235A, | PPKPKDTLMISRTPEVTCVVVDVSHEDPEVKFNWYVDGVEV |
| | F405L, M428L, | HNAKTKPREEQYNSTYRVVSVLTVLHQDWLNGKEYKCKVSN |
| | N434S mutations and | KALPAPIEKTISKAKGQPREPQVYTLPPSRDELTKNQVSLT |
| | G236 deleted (bolded) | CLVKGFYPSDIAVEWESNGQPENNYKTTPPVLDSDGSFLLY |
| | | SkLTVDKSRWQQGNVFSCSVLHEALHSHYTQKSLSLSPGK |
| 24 | anti-TNFα antibody | EVQLVESGGVVVQPGGSLRLSCAASGFDFADYAMHWVRQAP |
| | heavy chain EAC166 | GKGLEWVSAITWNGGHTDYADSVKGRFTISRDNSKNSLYLQ |
| | based on ADA-H1X | MNSLRTEDTALYYCAKVSYLSTASSLDYWGQGTLVTVSSAS |
| | variable domain | TKGPSVFPLAPSSKSTSGGTAALGCLVKDYFPEPVTVSWNS |
| | (underlined) with | GALTSGVHTFPAVLQSSGLYSLSSVVTVPSSSLGTQTYICN |
| | IgG1 Fc with E233P | VNHKPSNTKVDKKVEPKSCDKTHTCPPCPAPPAAGPSVFLF |
| | L234A, L235A, | PPKPKDTLMISRTPEVTCVVVDVSHEDPEVKFNWYVDGVEV |
| | F405L, M428L, | HNAKTKPREEQYNSTYRVVSVLTVLHQDWLNGKEYKCKVSN |
| | N434S mutations and | KALPAPIEKTISKAKGQPREPQVYTLPPSRDELTKNQVSLT |
| | G236 deleted (bolded) | CLVKGFYPSDIAVEWESNGQPENNYKTTPPVLDSDGSFLLY |
| | | SkLTVDKSRWQQGNVFSCSVLHEALHSHYTQKSLSLSPGK |
| 25 | anti-TNFα antibody | EVQLVESGGGLVQPGGSLRLSCAASGFDFADYAMHWVRQAP |
| | heavy chain EAC167 | GKGLEWVSAITWNGGHTDYADSVKGRFTISRDNSKNTLYLQ |
| | based on ADA-H2X | MNSLRAEDTAVYYCAKVSYLSTASSLDYWGQGTLVTVSSAS |
| | variable domain | TKGPSVFPLAPSSKSTSGGTAALGCLVKDYFPEPVTVSWNS |
| | (underlined) with | GALTSGVHTFPAVLQSSGLYSLSSVVTVPSSSLGTQTYICN |
| | IgG1 Fc with E233P, | VNHKPSNTKVDKKVEPKSCDKTHTCPPCPAPPAAGPSVFLF |
| | L234A, L235A, | PPKPKDTLMISRTPEVTCVVVDVSHEDPEVKFNWYVDGVEV |
| | F405L, M428L, | HNAKTKPREEQYNSTYRVVSVLTVLHQDWLNGKEYKCKVSN |
| | N434S mutations and | KALPAPIEKTISKAKGQPREPQVYTLPPSRDELTKNQVSLT |
| | G236 deleted (bolded) | CLVKGFYPSDIAVEWESNGQPENNYKTTPPVLDSDGSFLLY |
| | | SkLTVDKSRWQQGNVFSCSVLHEALHSHYTQKSLSLSPGK |
| 26 | anti-TNFα antibody | EVQLVESGGGLVQPGGSLRLSCAASGFDFADYAMHWVRQAP |
| | heavy chain EAC168 | GKGLVWVSAITWNGGHTDYADSVKGRFTISRDNAKNTLYLQ |
| | based on ADA-H3X | MNSLRAEDTAVYYCAKVSYLSTASSLDYWGQGTLVTVSSAS |
| | variable domain | TKGPSVFPLAPSSKSTSGGTAALGCLVKDYFPEPVTVSWNS |
| | (underlined) with | GALTSGVHTFPAVLQSSGLYSLSSVVTVPSSSLGTQTYICN |
| | IgG1 Fc with E233P, | VNHKPSNTKVDKKVEPKSCDKTHTCPPCPAPPAAGPSVFLF |
| | L234A, L235A, | PPKPKDTLMISRTPEVTCVVVDVSHEDPEVKFNWYVDGVEV |
| | F405L, M428L, | HNAKTKPREEQYNSTYRVVSVLTVLHQDWLNGKEYKCKVSN |
| | N434S mutations and | KALPAPIEKTISKAKGQPREPQVYTLPPSRDELTKNQVSLT |
| | G236 deleted (bolded) | CLVKGFYPSDIAVEWESNGQPENNYKTTPPVLDSDGSFLLY |
| | | SkLTVDKSRWQQGNVFSCSVLHEALHSHYTQKSLSLSPGK |
| 27 | anti-TNFα antibody | QVQLVESGGGVVQPGGSLRLSCAASGFDFADYAMHWVRQAP |
| | heavy chain EAC169 | GKGLEWVSAITWNGGHTDYADSVKGRFTISRDNSKNTLYLQ |
| | based on ADA-H4X | MNSLRAEDTAVYYCAKVSYLSTASSLDYWGQGTLVTVSSAS |
| | variable domain | TKGPSVFPLAPSSKSTSGGTAALGCLVKDYFPEPVTVSWNS |
| | (underlined) with | GALTSGVHTFPAVLQSSGLYSLSSVVTVPSSSLGTQTYICN |
| | IgG1 Fc with E233P, | VNHKPSNTKVDKKVEPKSCDKTHTCPPCPAPPAAGPSVFLF |
| | L234A, L235A, | PPKPKDTLMISRTPEVTCVVVDVSHEDPEVKFNWYVDGVEV |
| | F405L, M428L, | HNAKTKPREEQYNSTYRVVSVLTVLHQDWLNGKEYKCKVSN |
| | N434S mutations and | KALPAPIEKTISKAKGQPREPQVYTLPPSRDELTKNQVSLT |
| | G236 deleted (bolded) | CLVKGFYPSDIAVEWESNGQPENNYKTTPPVLDSDGSFLLY |
| | | SkLTVDKSRWQQGNVFSCSVLHEALHSHYTQKSLSLSPGK |
| 28 | anti-TNFα antibody | DIQMTQSPSSLSASVGDRVTITCRASQGIRNYLAWYQQKPG |
| | light chain EAC34 | KAPKLLIYAASTLQSGVPSRFSGSGSGTDFTLTISSLQPED |
| | based on ADA-L | VATYYCQRYNRAPYTFGQGTKVEIKRTVAAPSVFIFPPSDE |
| | variable domain | QLKSGTASVVCLLNNFYPREAKVQWKVDNALQSGNSQESVT |
| | (underlined) | EQDSKDSTYSLSSTLTLSKADYEKHKVYACEVTHQGLSSPV |
| | | TKSFNRGEC |
| 29 | anti-TNFα antibody | EIVMTQSPATLSVSPGERATLSCRASQGIRNYLAWYQQKPG |
| | light chain EAC127 | QAPRLLIYAASTLQSGIPARFSGSGSGTEFTLTISSLQSED |
| | based on ADA-L1 | FAVYYCQRYNRAPYTFGQGTKVEIKRTVAAPSVFIFPPSDE |
| | variable domain | QLKSGTASVVCLLNNFYPREAKVQWKVDNALQSGNSQESVT |
| | (underlined) | EQDSKDSTYSLSSTLTLSKADYEKHKVYACEVTHQGLSSPV |
| | | TKSFNRGEC |
| 30 | anti-TNFα antibody | DIVMTQSPDSLAVSLGERATINCRASQGIRNYLAWYQQKPG |
| | light chain EAC128 | QAPKLLIYAASTLQSGVPDRFSGSGSGTDFTLTISSLQAED |
| | based on ADA-L2 | VAVYYCQRYNRAPYTFGQGTKVEIKRTVAAPSVFIFPPSDE |
| | variable domain | QLKSGTASVVCLLNNFYPREAKVQWKVDNALQSGNSQESVT |
| | (underlined) | EQDSKDSTYSLSSTLTLSKADYEKHKVYACEVTHQGLSSPV |
| | | TKSFNRGEC |
| 31 | Heavy chain variable | QVQLVESGGGVVQPGRSLRLSCAFSGFSLSTSGMGVGWIRQ |
| | domain Ab5H3 of | APGKGLEWVAHIWWDGDESYADSVKGRFTISKDNSKNTVYL |
| | anti-IL1β antibody | QMNSLRAEDTAVYFCARNRYDPPWFVDWGQGTLVTVSS |
| 32 | Heavy chain variable | EVQLVESGGGLVQPGGSLRLSCAASGFTFSSFGMHWVRQAP |
| | domain Ab8H1 of | GKGLEWVAYISIGSYTVHYADSVKGRFTISRDNAKNSLYLQ |
| | anti-IL1β antibody | MNSLRDEDTAVYYCVRDDYDVTDYTMDYWGQGTTVTVSS |
| 33 | Heavy chain variable | QVTLKESGPALVKPTQTLTLTCTFSGFSLSTSGMGVSWIRQ |
| | domain Ab9H1 of | PPGKGLEWLAHIYWDDDKYYSPSLKSRLTITKDTSKNQVVL |
| | anti-IL1β antibody | TMTNMDPVDTATYYCARGSYDPSPFDYWGQGTTVTVSS |
| 34 | Light chain variable | DIQMTQSTSSLSASVGDRVTITCRASQDISNYLSWYQQKPG |
| | domain Ab5L of anti- | KAVKLLIYYTSKLHSGVPSRFSGSGSGTDYTLTISSLQQED |
| | IL1β antibody | FATYFCLQGKMLPWTFGQGTKLEIK |
| 35 | Light chain variable | DIVMTQTPLSLPVTPGEPASISCKSSQSLLNSRTRKNYLAW |
| | domain Ab8L3 of | YLQKPGQSPQLLIYWASTRESGVPDRFSGSGSGTDFTLKIS |
| | anti-IL1β antibody | RVEAEDVGVYYCKQTYNFPTFGQGTKLEIK |
| 36 | Light chain variable | DIQMTQSPSSLSASVGDRVTITCRPSRDITNYLNWYQQKPG |
| | domain Ab9L1 of | KTLKLLIYHTSRLHSGVPSRFSGSGSGTDYTLTISSLQPED |
| | anti-IL1β antibody | FATYFCQQSKSVPWTFGGGTKVEIK |
| 37 | anti-IL1β antibody | QVQLVESGGGVVQPGRSLRLSCAFSGFSLSTSGMGVGWIRQ |
| | heavy chain EAC53 | APGKGLEWVAHIWWDGDESYADSVKGRFTISKDNSKNTVYL |
| | based on Ab5H3 | QMNSLRAEDTAVYFCARNRYDPPWFVDWGQGTLVTVSSAST |
| | variable domain | KGPSVFPLAPSSKSTSGGTAALGCLVKDYFPEPVTVSWNSG |
| | (underlined) with | ALTSGVHTFPAVLQSSGLYSLSSVVTVPSSSLGTQTYICNV |
| | IgG1 Fc with K409R | NHKPSNTKVDKKVEPKSCDKTHTCPPCPAPELLGGPSVFLF |
| | mutation (bolded) | PPKPKDTLMISRTPEVTCVVVDVSHEDPEVKFNWYVDGVEV |
| | | HNAKTKPREEQYNSTYRVVSVLTVLHQDWLNGKEYKCKVSN |
| | | KALPAPIEKTISKAKGQPREPQVYTLPPSREEMTKNQVSLT |
| | | CLVKGFYPSDIAVEWESNGQPENNYKTTPPVLDSDGSFFLY |
| | | SRLTVDKSRWQQGNVFSCSVMHEALHNHYTQKSLSLSPGK |
| 38 | anti-IL1β antibody | EVQLVESGGGLVQPGGSLRLSCAASGFTFSSFGMHWVRQAP |
| | heavy chain EAC73 | GKGLEWVAYISIGSYTVHYADSVKGRFTISRDNAKNSLYLQ |
| | based on Ab8H1 | MNSLRDEDTAVYYCVRDDYDVTDYTMDYWGQGTTVTVSSAS |
| | variable domain | TKGPSVFPLAPSSKSTSGGTAALGCLVKDYFPEPVTVSWNS |
| | (underlined) with | GALTSGVHTFPAVLQSSGLYSLSSVVTVPSSSLGTQTYICN |
| | IgG1 Fc with K409R | VNHKPSNTKVDKKVEPKSCDKTHTCPPCPAPELLGGPSVFL |
| | mutation (bolded) | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSREEMTKNQVSL |
| | | TCLVKGFYPSDIAVEWESNGQPENNYKTTPPVLDSDGSFFL |
| | | YSRLTVDKSRWQQGNVFSCSVMHEALHNHYTQKSLSLSPGK |
| 39 | anti-IL1β antibody | QVTLKESGPALVKPTQTLTLTCTFSGFSLSTSGMGVSWIRQ |
| | heavy chain EAC80 | PPGKGLEWLAHIYWDDDKYYSPSLKSRLTITKDTSKNQVVL |
| | based on Ab9H1 | TMTNMDPVDTATYYCARGSYDPSPFDYWGQGTTVTVSSAST |
| | variable domain | KGPSVFPLAPSSKSTSGGTAALGCLVKDYFPEPVTVSWNSG |
| | (underlined) with | ALTSGVHTFPAVLQSSGLYSLSSVVTVPSSSLGTQTYICNV |
| | IgG1 Fc with K409R | NHKPSNTKVDKKVEPKSCDKTHTCPPCPAPELLGGPSVFLF |
| | mutation (bolded) | PPKPKDTLMISRTPEVTCVVVDVSHEDPEVKFNWYVDGVEV |
| | | HNAKTKPREEQYNSTYRVVSVLTVLHQDWLNGKEYKCKVSN |
| | | KALPAPIEKTISKAKGQPREPQVYTLPPSREEMTKNQVSLT |
| | | CLVKGFYPSDIAVEWESNGQPENNYKTTPPVLDSDGSFFLY |
| | | SRLTVDKSRWQQGNVFSCSVMHEALHNHYTQKSLSLSPGK |
| 40 | anti-IL1β antibody | QVQLVESGGGVVQPGRSLRLSCAFSGFSLSTSGMGVGWIRQ |
| | heavy chain EAC120 | APGKGLEWVAHIWWDGDESYADSVKGRFTISKDNSKNTVYL |
| | based on Ab5H3 | QMNSLRAEDTAVYFCARNRYDPPWFVDWGQGTLVTVSSAST |
| | variable domain | KGPSVFPLAPSSKSTSGGTAALGCLVKDYFPEPVTVSWNSG |
| | (underlined) with | ALTSGVHTFPAVLQSSGLYSLSSVVTVPSSSLGTQTYICNV |
| | IgG1 Fc with L234A, | NHKPSNTKVDKKVEPKSCDKTHTCPPCPAPEAAGGPSVFLF |
| | L235A, K409R, | PPKPKDTLMISRTPEVTCVVVDVSHEDPEVKFNWYVDGVEV |
| | M428L, N434S | HNAKTKPREEQYNSTYRVVSVLTVLHQDWLNGKEYKCKVSN |
| | mutations (bolded) | KALPAPIEKTISKAKGQPREPQVYTLPPSRDELTKNQVSLT |
| | | CLVKGFYPSDIAVEWESNGQPENNYKTTPPVLDSDGSFFLY |
| | | SRLTVDKSRWQQGNVFSCSVLHEALHSHYTQKSLSLSPGK |
| 41 | anti-IL1β antibody | EVQLVESGGGLVQPGGSLRLSCAASGFTFSSFGMHWVRQAP |
| | heavy chain EAC121 | GKGLEWVAYISIGSYTVHYADSVKGRFTISRDNAKNSLYLQ |
| | based on Ab8H1 | MNSLRDEDTAVYYCVRDDYDVTDYTMDYWGQGTTVTVSSAS |
| | variable domain | TKGPSVFPLAPSSKSTSGGTAALGCLVKDYFPEPVTVSWNS |
| | (underlined) with | GALTSGVHTFPAVLQSSGLYSLSSVVTVPSSSLGTQTYICN |
| | IgG1 Fc with L234A, | VNHKPSNTKVDKKVEPKSCDKTHTCPPCPAPEAAGGPSVFL |
| | L235A, K409R, | FPPKPKDTLMISRTPEVTCVVVDVSHEDPEVKFNWYVDGVE |
| | M428L, N434S | VHNAKTKPREEQYNSTYRVVSVLTVLHQDWLNGKEYKCKVS |
| | mutations (bolded) | NKALPAPIEKTISKAKGQPREPQVYTLPPSRDELTKNQVSL |
| | | TCLVKGFYPSDIAVEWESNGQPENNYKTTPPVLDSDGSFFL |
| | | YSRLTVDKSRWQQGNVFSCSVLHEALHSHYTQKSLSLSPGK |
| 42 | anti-IL1β antibody | EVQLVESGGGLVQPGGSLRLSCAASGFTFSSFGMHWVRQAP |
| | heavy chain EAC145 | GKGLEWVAYISIGSYTVHYADSVKGRFTISRDNAKNSLYLQ |
| | based on Ab8H1 | MNSLRDEDTAVYYCVRDDYDVTDYTMDYWGQGTTVTVSSAS |
| | variable domain | TKGPSVFPLAPSSKSTSGGTAALGCLVKDYFPEPVTVSWNS |
| | (underlined) with | GALTSGVHTFPAVLQSSGLYSLSSVVTVPSSSLGTQTYICN |
| | IgG1 Fc with E233P | VNHKPSNTKVDKKVEPKSCDKTHTCPPCPAPPAAGPSVFLF |
| | L234A, L235A, | PPKPKDTLMISRTPEVTCVVVDVSHEDPEVKFNWYVDGVEV |
| | K409R, M428L, | HNAKTKPREEQYNSTYRVVSVLTVLHQDWLNGKEYKCKVSN |
| | N434S mutations and | KALPAPIEKTISKAKGQPREPQVYTLPPSRDELTKNQVSLT |
| | G236 deleted (bolded) | CLVKGFYPSDIAVEWESNGQPENNYKTTPPVLDSDGSFFLY |
| | | SRLTVDKSRWQQGNVFSCSVLHEALHSHYTQKSLSLSPGK |
| 43 | anti-IL1β antibody | QVTLKESGPALVKPTQTLTLTCTFSGFSLSTSGMGVSWIRQ |
| | heavy chain EAC161 | PPGKGLEWLAHIYWDDDKYYSPSLKSRLTITKDTSKNQVVL |
| | based on Ab9H1 | TMTNMDPVDTATYYCARGSYDPSPFDYWGQGTTVTVSSAST |
| | variable domain | KGPSVFPLAPSSKSTSGGTAALGCLVKDYFPEPVTVSWNSG |
| | (underlined) with | ALTSGVHTFPAVLQSSGLYSLSSVVTVPSSSLGTQTYICNV |
| | IgG1 Fc with E233P, | NHKPSNTKVDKKVEPKSCDKTHTCPPCPAPPAAGPSVFLFP |
| | L234A, L235A, | PKPKDTLMISRTPEVTCVVVDVSHEDPEVKFNWYVDGVEVH |
| | K409R, M428L, | NAKTKPREEQYNSTYRVVSVLTVLHQDWLNGKEYKCKVSNK |
| | N434S mutations and | ALPAPIEKTISKAKGQPREPQVYTLPPSRDELTKNQVSLTC |
| | G236 deleted (bolded) | LVKGFYPSDIAVEWESNGQPENNYKTTPPVLDSDGSFFLYS |
| | | RLTVDKSRWQQGNVFSCSVLHEALHSHYTQKSLSLSPGK |
| 44 | anti-IL1β antibody | DIQMTQSTSSLSASVGDRVTITCRASQDISNYLSWYQQKPG |
| | light chain EAC32 | KAVKLLIYYTSKLHSGVPSRFSGSGSGTDYTLTISSLQQED |
| | based on Ab5L | FATYFCLQGKMLPWTFGQGTKLEIKRTVAAPSVFIFPPSDE |
| | variable domain | QLKSGTASVVCLLNNFYPREAKVQWKVDNALQSGNSQESVT |
| | (underlined) | EQDSKDSTYSLSSTLTLSKADYEKHKVYACEVTHQGLSSPV |
| | | TKSFNRGEC |
| 45 | anti-IL1β antibody | DIVMTQTPLSLPVTPGEPASISCKSSQSLLNSRTRKNYLAW |
| | light chain EAC78 | YLQKPGQSPQLLIYWASTRESGVPDRFSGSGSGTDFTLKIS |
| | based on Ab8L3 | RVEAEDVGVYYCKQTYNFPTFGQGTKLEIKRTVAAPSVFIF |
| | variable domain | PPSDEQLKSGTASVVCLLNNFYPREAKVQWKVDNALQSGNS |
| | (underlined) | QESVTEQDSKDSTYSLSSTLTLSKADYEKHKVYACEVTHQG |
| | | LSSPVTKSFNRGEC |
| 46 | anti-IL1β antibody | DIQMTQSPSSLSASVGDRVTITCRPSRDITNYLNWYQQKPG |
| | light chain EAC83 | KTLKLLIYHTSRLHSGVPSRFSGSGSGTDYTLTISSLQPED |
| | based on Ab9L1 | FATYFCQQSKSVPWTFGGGTKVEIKRTVAAPSVFIFPPSDE |
| | variable domain | QLKSGTASVVCLLNNFYPREAKVQWKVDNALQSGNSQESVT |
| | (underlined) | EQDSKDSTYSLSSTLTLSKADYEKHKVYACEVTHQGLSSPV |
| | | TKSFNRGEC |
| 47 | IgG1 Fc | KVEPKSCDKTHTCPPCPAPELLGGPSVFLFPPKPKDTLMIS |
| | | RTPEVTCVVVDVSHEDPEVKFNWYVDGVEVHNAKTKPREEQ |
| | | YNSTYRVVSVLTVLHQDWLNGKEYKCKVSNKALPAPIEKTI |
| | | SKAKGQPREPQVYTLPPSRDELTKNQVSLTCLVKGFYPSDI |
| | | AVEWESNGQPENNYKTTPPVLDSDGSFFLYSKLTVDKSRWQ |
| | | QGNVFSCSVMHEALHNHYTQKSLSLSPGK |
| 48 | IgG2 Fc | TVERKCCVECPPCPAPPVAGPSVFLFPPKPKDTLMISRTPE |
| | | VTCVVVDVSHEDPEVQFNWYVDGVEVHNAKTKPREEQFNST |
| | | FRVVSVLTVVHQDWLNGKEYKCKVSNKGLPAPIEKTISKTK |
| | | GQPREPQVYTLPPSREEMTKNQVSLTCLVKGFYPSDIAVEW |
| | | ESNGQPENNYKTTPPMLDSDGSFFLYSKLTVDKSRWQQGNV |
| | | FSCSVMHEALHNHYTQKSLSLSPGK |
| 49 | IgG3 Fc | RVELKTPLGDTTHTCPPCPAPELLGGPSVFLFPPKPKDTLM |
| | | ISRTPEVTCVVVDVSHEDPEVKFNWYVDGVEVHNAKTKPRE |
| | | EQYNSTYRVVSVLTVLHQDWLNGKEYKCKVSNKALPAPIEK |
| | | TISKAKGQPREPQVYTLPPSRDELTKNQVSLTCLVKGEYPS |
| | | DIAVEWESNGQPENNYKTTPPVLDSDGSFFLYSKLTVDKSR |
| | | WQQGNVFSCSVMHEALHNHYTQKSLSLSPGK |
| 50 | IgG4 Fc | RVESKYGPPCPSCPAPEFLGGPSVFLFPPKPKDTLMISRTP |
| | | EVTCVVVDVSQEDPEVQFNWYVDGVEVHNAKTKPREEQFNS |
| | | TYRVVSVLTVLHQDWLNGKEYKCKVSNKGLPSSIEKTISKA |
| | | KGQPREPQVYTLPPSQEEMTKNQVSLTCLVKGEYPSDIAVE |
| | | WESNGQPENNYKTTPPVLDSDGSFFLYSRLTVDKSRWQEGN |
| | | VFSCSVMHEALHNHYTQKSLSLSLGK |
| 51 | IgG₁ F_{c} with | KVEPKSCDKTHTCPPCPAPELLGGPSVFLFPPKPKDTLYIT |
| | M252Y/S254T/T256E | REPEVTCVVVDVSHEDPEVKFNWYVDGVEVHNAKTKPREEQ |
| | mutations | YNSTYRVVSVLTVLHQDWLNGKEYKCKVSNKALPAPIEKTI |
| | | SKAKGQPREPQVYTLPPSRDELTKNQVSLTCLVKGFYPSDI |
| | | AVEWESNGQPENNYKTTPPVLDSDGSFFLYSKLTVDKSRWQ |
| | | QGNVFSCSVMHEALHNHYTQKSLSLSPGK |
| 52 | IgG₁ F_{c} with | KVEPKSCDKTHTCPPCPAPELLGGPSVFLFPPKPKDTLMIS |
| | M428L/N434S | RTPEVTCVVVDVSHEDPEVKFNWYVDGVEVHNAKTKPREEQ |
| | mutations | YNSTYRVVSVLTVLHQDWLNGKEYKCKVSNKALPAPIEKTI |
| | | SKAKGQPREPQVYTLPPSRDELTKNQVSLTCLVKGEYPSDI |
| | | AVEWESNGQPENNYKTTPPVLDSDGSFFLYSKLTVDKSRWQ |
| | | QGNVFSCSVLHEALHSHYTQKSLSLSPGK |
| 53 | IgG₁ F_{c} with | KVEPKSCDKTHTCPPCPAPELLGGPSVFLFPPKPKDQLMIS |
| | T250Q/M428L | RTPEVTCVVVDVSHEDPEVKFNWYVDGVEVHNAKTKPREEQ |
| | mutations | YNSTYRVVSVLTVLHQDWLNGKEYKCKVSNKALPAPIEKTI |
| | | SKAKGQPREPQVYTLPPSRDELTKNQVSLTCLVKGEYPSDI |
| | | AVEWESNGQPENNYKTTPPVLDSDGSFFLYSKLTVDKSRWQ |
| | | QGNVFSCSVLHEALHNHYTQKSLSLSPGK |
| 54 | IgG₁ F_{c} with | KVEPKSCDKTHTCPPCPAPELLGGPSVFLFPPKPKDTLMIS |
| | N434A | RTPEVTCVVVDVSHEDPEVKFNWYVDGVEVHNAKTKPREEQ |
| | mutations | YNSTYRVVSVLTVLHQDWLNGKEYKCKVSNKALPAPIEKTI |
| | | SKAKGQPREPQVYTLPPSRDELTKNQVSLTCLVKGEYPSDI |
| | | AVEWESNGQPENNYKTTPPVLDSDGSFFLYSKLTVDKSRWQ |
| | | QGNVFSCSVMHEALHAHYTQKSLSLSPGK |
| 55 | IgG₁ F_{c} with | KVEPKSCDKTHTCPPCPAPELLGGPSVFLFPPKPKDTLMIS |
| | T307A/E380A/N434A | RTPEVTCVVVDVSHEDPEVKFNWYVDGVEVHNAKTKPREEQ |
| | mutations | YNSTYRVVSVLAVLHQDWLNGKEYKCKVSNKALPAPIEKTI |
| | | SKAKGQPREPQVYTLPPSRDELTKNQVSLTCLVKGFYPSDI |
| | | AVAWESNGQPENNYKTTPPVLDSDGSFFLYSKLTVDKSRWQ |
| | | QGNVFSCSVMHEALHAHYTQKSLSLSPGK |
| 56 | IgG₁ F_{c} with | KVEPKSCDKTHTCPPCPAPPAAGPSVFLFPPKPKDTLMISR |
| | E233P/L234A/L235A | TPEVTCVVVDVSHEDPEVKFNWYVDGVEVHNAKTKPREEQY |
| | mutations and G236 | NSTYRVVSVLTVLHQDWLNGKEYKCKVSNKALPAPIEKTIS |
| | deleted | KAKGQPREPQVYTLPPSRDELTKNQVSLTCLVKGFYPSDIA |
| | | VEWESNGQPENNYKTTPPVLDSDGSFFLYSKLTVDKSRWQQ |
| | | GNVFSCSVMHEALHNHYTQKSLSLSPGK |
| 57 | IgG₁ F_{c} with F405L | KVEPKSCDKTHTCPPCPAPELLGGPSVFLEPPKPKDTLMIS |
| | mutation | RTPEVTCVVVDVSHEDPEVKFNWYVDGVEVHNAKTKPREEQ |
| | | YNSTYRVVSVLTVLHQDWLNGKEYKCKVSNKALPAPIEKTI |
| | | SKAKGQPREPQVYTLPPSRDELTKNQVSLTCLVKGFYPSDI |
| | | AVEWESNGQPENNYKTTPPVLDSDGSFLLYSKLTVDKSRWQ |
| | | QGNVFSCSVMHEALHNHYTQKSLSLSPGK |
| 58 | IgG₁ F_{c} with K409R | KVEPKSCDKTHTCPPCPAPELLGGPSVFLEPPKPKDTLMIS |
| | mutation | RTPEVTCVVVDVSHEDPEVKFNWYVDGVEVHNAKTKPREEQ |
| | | YNSTYRVVSVLTVLHQDWLNGKEYKCKVSNKALPAPIEKTI |
| | | SKAKGQPREPQVYTLPPSRDELTKNQVSLTCLVKGFYPSDI |
| | | AVEWESNGQPENNYKTTPPVLDSDGSFFLYSRLTVDKSRWQ |
| | | QGNVFSCSVMHEALHNHYTQKSLSLSPGK |
| 59 | Polynucleotide | gaggtgcagctggtggagagcggcggaggactggtgcagcc |
| | sequence encoding | cggtagatctttaagactgagctgtgccgccagcggcttca |
| | EAC33 | cattcgacgactacgccatgcactgggtgagacaagctccc |
| | | ggtaaaggtttagaatgggtgagcgccatcacttggaacag |
| | | cggccacatcgactacgccgacagcgtggagggtcgtttca |
| | | ccatctctcgtgacaacgccaagaactctttatatttacag |
| | | atgaactctttaagagccgaggacaccgccgtgtactactg |
| | | cgccaaggtgagctatttaagcaccgccagctctttagact |
| | | actggggccaaggtactttagtgaccgtgagcagcgccagc |
| | | accaagggcccatcggtcttccccctggcaccctcctccaa |
| | | gagcacctctgggggcacagcggccctgggctgcctggtca |
| | | aggactacttccccgaaccggtgacggtgtcgtggaactca |
| | | ggcgccctgaccagcggcgtgcacaccttcccggctgtcct |
| | | acagtcctcaggactctactccctcagcagcgtggtgaccg |
| | | tgccctccagcagcttgggcacccagacctacatctgcaac |
| | | gtgaatcacaagcccagcaacaccaaggtggacaagaaagt |
| | | tgagcccaaatcttgtgacaaaactcacacatgcccaccgt |
| | | gcccagcacctgaactcctggggggaccgtcagtatcctct |
| | | tccccccaaaacccaaggacaccctcatgatctcccggacc |
| | | cctgaggtcacatgcgtggtggtggacgtgagccacgaaga |
| | | ccctgaggtcaagttcaactggtacgtggacggcgtggagg |
| | | tgcataatgccaagacaaagccgcgggaggagcagtacaac |
| | | agcacgtaccgtgtggtcagcgtcctcaccgtcctgcacca |
| | | ggactggctgaatggcaaggagtacaagtgcaaggtctcca |
| | | acaaagccctcccagcccccatcgagaaaaccatctccaaa |
| | | gccaaagggcagccccgagaaccacaggtgtacaccctgcc |
| | | cccatcccgggaggagatgaccaagaaccaggtcagcctga |
| | | cctgcctggtcaaaggcttctatcccagcgacatcgccgtg |
| | | gagtgggagagcaatgggcagccggagaacaactacaagac |
| | | cacgcctcccgtgctggactccgacggctccttcttgctct |
| | | acagcaagctcaccgtggacaagagcaggtggcagcagggg |
| | | aacgtcttctcatgctccgtgatgcatgaggctctgcacaa |
| | | ccactacacgcagaagagcctctccctgtctccgggtaaa |
| 60 | Polynucleotide | gacatccagatgacccagagccctagctctttaagcgctag |
| | sequence encoding | cgtgggcgatcgtgtgaccatcacttgtcgtgccagccaag |
| | EAC34 | gtattcgtaactatttagcttggtaccagcagaagcccggc |
| | | aaggcccccaagctgctgatctacgccgccagcactttaca |
| | | gagcggagtgcctagcagatttagcggcagcggtagcggca |
| | | ccgatttcactttaaccatcagctctttacagcccgaagac |
| | | gtggccacctactactgccagaggtacaatcgtgcccccta |
| | | cacctttggccaaggtaccaaggtggagatcaagcgtacgg |
| | | tggctgcaccatctgtcttcatcttcccgccatctgatgag |
| | | cagttgaaatctggaactgcctctgttgtgtgcctgctgaa |
| | | taacttctatcccagagaggccaaagtacagtggaaggtgg |
| | | ataacgccctccaatcgggtaactcccaggagagtgtcaca |
| | | gagcaggacagcaaggacagcacctacagcctcagcagcac |
| | | cctgacgctgagcaaagcagactacgagaaacacaaagtct |
| | | acgcctgcgaagtcacccatcagggcctgagctcgcccgtc |
| | | acaaagagatcaacaggggagagtgt |
| 61 | Polynucleotide | caagttcagctggtggagagcggaggaggcgtggtgcagcc |
| | sequence encoding | cggtagatctttaaggctgagctgcgccttcagcggcttct |
| | EAC53 | ctttaagcaccagcggaatgggcgtgggctggatcagacaa |
| | | gctcccggaaagggtttagagtgggtggcccacatctggtg |
| | | ggacggcgacgagagctacgccgacagcgtgaagggtcgtt |
| | | tcaccatcagcaaggacaactccaagaacaccgtgtattta |
| | | cagatgaactctttaagggccgaggacaccgccgtgtactt |
| | | ctgcgctcgtaatcgttacgaccccccttggtttgtggact |
| | | ggggccaaggtactttagtgacagtgagcagcgccagcacc |
| | | aagggcccatcggtcttccccctggcaccctcctccaagag |
| | | cacctctgggggcacagcggccctgggctgcctggtcaagg |
| | | actacttccccgaaccggtgacggtgtcgtggaactcaggc |
| | | gccctgaccagcggcgtgcacaccttcccggctgtcctaca |
| | | gtcctcaggactctactccctcagcagcgtggtgaccgtgc |
| | | cctccagcagcttgggcacccagacctacatctgcaacgtg |
| | | aatcacaagcccagcaacaccaaggtggacaagaaagttga |
| | | gcccaaatcttgtgacaaaactcacacatgcccaccgtgcc |
| | | cagcacctgaactcctggggggaccgtcagtatcctcttcc |
| | | ccccaaaacccaaggacaccctcatgatctcccggacccct |
| | | gaggtcacatgcgtggtggtggacgtgagccacgaagaccc |
| | | tgaggtcaagttcaactggtacgtggacggcgtggaggtgc |
| | | ataatgccaagacaaagccgcgggaggagcagtacaacagc |
| | | acgtaccgtgtggtcagcgtcctcaccgtcctgcaccagga |
| | | ctggctgaatggcaaggagtacaagtgcaaggtctccaaca |
| | | aagccctcccagcccccatcgagaaaaccatctccaaagcc |
| | | aaagggcagccccgagaaccacaggtgtacaccctgccccc |
| | | atcccgggaggagatgaccaagaaccaggtcagcctgacct |
| | | gcctggtcaaaggcttctatcccagcgacatcgccgtggag |
| | | tgggagagcaatgggcagccggagaacaactacaagaccac |
| | | gcctcccgtgctggactccgacggctccttcttcctctaca |
| | | gcagactcaccgtggacaagagcaggtggcagcaggggaac |
| | | gtcttctcatgctccgtgatgcatgaggctctgcacaacca |
| | | ctacacgcagaagagcctctccctgtctccgggtaaa |
| 62 | Polynucleotide | gacatccagatgacccagagcacatcctctttatccgccag |
| | sequence encoding | cgtgggcgacagagtgaccatcacttgtcgtgccagccaag |
| | EAC32 | atatcagcaactatttaagctggtaccagcagaagcccggc |
| | | aaggccgtgaagctgctgatctactacaccagcaagctgca |
| | | cagcggcgtgcctagcagattcagcggcagcggaagcggca |
| | | ccgactacactttaaccatcagctctttacagcaagaagac |
| | | ttcgccacctacttctgtttacaaggtaagatgctgccttg |
| | | gaccttcggccaaggtaccaagctggagatcaagcgtacgg |
| | | tggctgcaccatctgtcttcatcttcccgccatctgatgag |
| | | cagttgaaatctggaactgcctctgttgtgtgcctgctgaa |
| | | taacttctatcccagagaggccaaagtacagtggaaggtgg |
| | | ataacgccctccaatcgggtaactcccaggagagtgtcaca |
| | | gagcaggacagcaaggacagcacctacagcctcagcagcac |
| | | cctgacgctgagcaaagcagactacgagaaacacaaagtct |
| | | acgcctgcgaagtcacccatcagggcctgagctcgcccgtc |
| | | acaaagagatcaacaggggagagtgt |

### REFERENCES

Aggarwal, B. B. (2003). “Signalling pathways of the TNF superfamily: a double-edged sword.” Nat Rev Immunol 3(9): 745-756.
Bendele A. M., E. S. Chlipala, J. Scherrer, et. al., (2002) “Combination benefit of treatment with the cytokine inhibitors interleukin-1 receptor antagonist and pegylated soluble tumor necrosis factor receptor type I in animal models of rheumatoid arthritis”. Arthritis Rheumatol. 43(12):2648-2659.
Bolt, S., E. Routledge, I. Lloyd, L. Chatenoud, H. Pope, S. D. Gorman, M. Clark and H. Waldmann (1993). “The generation of a humanized, non-mitogenic CD3 monoclonal antibody which retains in vitro immunosuppressive properties.” Eur J Immunol 23(2): 403-411.
Chu, S. Y., I. Vostiar, S. Karki, G. L. Moore, G. A. Lazar, E. Pong, P. F. Joyce, D. E. Szymkowski and J. R. Desjarlais (2008). “Inhibition of B cell receptor-mediated activation of primary human B cells by coengagement of CD19 and FcgammaRIIb with Fc-engineered antibodies.” Mol Immunol 45(15): 3926-3933.
Dall'Acqua, W. F., P. A. Kiener and H. Wu (2006). “Properties of human IgG1s engineered for enhanced binding to the neonatal Fc receptor (FcRn).” J Biol Chem 281(33): 23514-23524.
Erekat, N. and M. D. Al-Jarrah (2018). “Interleukin-1 Beta and Tumor Necrosis Factor Alpha Upregulation and Nuclear Factor Kappa B Activation in Skeletal Muscle from a Mouse Model of Chronic/Progressive Parkinson Disease.” Med Sci Monit 24: 7524-7531.
Feldmann, M., F. M. Brennan, E. Paleolog, A. Cope, P. Taylor, R. Williams, J. Woody and R. N. Maini (2004). “Anti-TNFalpha therapy of rheumatoid arthritis: what can we learn about chronic disease?” Novartis Found Symp 256: 53-69; discussion 69-73, 106-111, 266-109.
Gram, H. (2016). “Preclinical characterization and clinical development of ILARIS((R)) (canakinumab) for the treatment of autoinflammatory diseases.” Curr Opin Chem Biol 32: 1-9.
Hess, A., R. Axmann, J. Rech, S. Finzel, C. Heindl, S. Kreitz, M. Sergeeva, M. Saake, M. Garcia, G. Kollias, R. H. Straub, O. Sporns, A. Doerfler, K. Brune and G. Schett (2011). “Blockade of TNF-alpha rapidly inhibits pain responses in the central nervous system.” Proc Natl Acad Sci USA 108(9): 3731-3736.
Hezareh, M., A. J. Hessell, R. C. Jensen, J G. Van de Winkel and P. W. Parren. (2001). “Effector function activities of a panel of mutants of a broadly neutralizing antibody against human immunodeficiency virus Type 1.” J Virol. 75(24):1261-8.
Hinton, P. R., J. M. Xiong, M. G. Johlfs, M. T. Tang, S. Keller and N. Tsurushita (2006). “An engineered human IgG1 antibody with longer serum half-life.” J Immunol 176(1): 346-356.
Huang, C. C., C. H. Chiou, S. C. Liu, S. L. Hu, C. M. Su, C. H. Tsai and C. H. Tang (2019). “Melatonin attenuates TNF-alpha and IL-1beta expression in synovial fibroblasts and diminishes cartilage degradation: Implications for the treatment of rheumatoid arthritis.” J Pineal Res 66(3): e12560.
Issafras, H., J. A. Corbin, I. D. Goldfine and M. K. Roell (2013). “Detailed mechanistic analysis of gevokizumab, an allosteric anti-IL-1beta antibody with differential receptor-modulating properties.” J Pharmacol Exp Ther 348(1): 202-215.
Jani, M., W. G. Dixon and H. Chinoy (2018). “Drug safety and immunogenicity of tumour necrosis factor inhibitors: the story so far.” Rheumatology 57(11): 1896-1907.
Kaymakcalan, Z., P. Sakorafas, S. Bose, S. Scesney, L. Xiong, D. K. Hanzatian, J. Salfeld and E. H. Sasso (2009). “Comparisons of affinities, avidities, and complement activation of adalimumab, infliximab, and etanercept in binding to soluble and membrane tumor necrosis factor.” Clin Immunol 131(2): 308-316.
Kinder, M., A. R. Greenplate, K. D. Grugan, K. L. Soring, K. A. Heeringa, S. G. McCarthy, G. Bannish, M. Perpetua, F. Lynch, R. E. Jordan, W. R. Strohl and R. J. Brerski (2013). “Engineered protease-resistant antibodies with selectable cell-killing functions.” J Biol Chem 288(43): 30843-30854.
Kobayashi, S. S., S. Vali, A. Kumar, N. Singh, T. Abbasi and P. P. Sayeski (2016). “Identification of myeloproliferative neoplasm drug agents via predictive simulation modeling: assessing responsiveness with micro-environment derived cytokines.” Oncotarget 7(24): 35989-36001.
Kuratnik, A., V. E. Senapati, R. Verma, B. G. Mellone, A. T. Vella and C. Giardina (2012). “Acute sensitization of colon cancer cells to inflammatory cytokines by prophase arrest.” Biochem Pharmacol 83(9): 1217-1228.
Labrijn, A. F., M. L. Janmaat, J. M. Reichert and P. Parren (2019). “Bispecific antibodies: a mechanistic review of the pipeline.” Nat Rev Drug Discov.
Labrijn, A. F., J. I. Meesters, B. E. de Goeij, E. T. van den Bremer, J. Neijssen, M. D. van Kampen, K. Strumane, S. Verploegen, A. Kundu, M. J. Gramer, P. H. van Berkel, J. G. van de Winkel, J. Schuurman and P. W. Parren (2014). “Efficient generation of stable bispecific IgG1 by controlled Fab-arm exchange.” Proc Natl Acad Sci USA 110(13): 5145-5150.
Leal, M. C., J. C. Casabona, M. Puntel and F. J. Pitossi (2013). “Interleukin-1beta and tumor necrosis factor-alpha: reliable targets for protective therapies in Parkinson's Disease?” Front Cell Neurosci 7: 53.
Li, Y., Y. Zhang, D. B. Liu, H. Y. Liu, W. G. Hou and Y. S. Dong (2013). “Curcumin attenuates diabetic neuropathic pain by downregulating TNF-alpha in a rat model.” Int J Med Sci 10(4): 377-381.
Lou, X., Y. Hou and D. Liang (2013). “Effects of hepatitis B virus X protein on human T cell cytokines.” Can J Microbiol 59(9): 620-626.
Mantzaris, G. J. (2016). “Anti-TNFs: Originators and Biosimilars.” Dig Dis 34(1-2): 132-139.
Martinez-Reza, I., L. Diaz, D. Barrera, M. Segovia-Mendoza, S. Pedraza-Sanchez, G. Soca-Chafre, F. Larrea and R. Garcia-Becerra (2019). “Calcitriol Inhibits the Proliferation of Triple-Negative Breast Cancer Cells through a Mechanism Involving the Proinflammatory Cytokines IL-1beta and TNF-alpha.” J Immunol Res 2019: 6384278.
McCarty, S. and W. Frishman (2014). “Interleukin 1beta: a proinflammatory target for preventing atherosclerotic heart disease.” Cardiol Rev 22(4): 176-181.
Monaco, C., J. Nanchahal, P. Taylor and M. Feldmann (2015). “Anti-TNF therapy: past, present and future.” Int Immunol 27(1): 55-62.
Moore, A., S. Allden, T. Bourne, M. C. Denis, K. Kranidioti, R. Okoye, Y. Sotsios, Z. Stencel, A. Vugler, G. Watt, et al. (2014). “Collagen II antibody-induced arthritis in Tg1278TNFKO mice: optimization of a novel model to assess treatments targeting human TNFα in rheumatoid arthritis”. J Transl Med. 12(1):285.
Moots, R. J., C. Curiale, D. Petersel, C. Rolland, H. Jones and E. Mysler (2018). “Efficacy and Safety Outcomes for Originator TNF Inhibitors and Biosimilars in Rheumatoid Arthritis and Psoriasis Trials: A Systematic Literature Review.” BioDrugs 32(3): 193-199.
Murphy, J. M., K. Jeong, Y. A. R. Rodriguez, J. H. Kim, E. E. Ahn and S. S. Lim (2019). “FAK and Pyk2 activity promote TNF-alpha and IL-1beta-mediated pro-inflammatory gene expression and vascular inflammation.” Sci Rep 9(1): 7617.
Owczarczyk-Saczonek, A., W. Owczarek, A. Osmola-Mankowska, Z. Adamski, W. Placek and A. Rakowska (2019). “Secondary failure of TNF-alpha inhibitors in clinical practice.” Dermatol Ther 32(1): e12760.
Petkova, S. B., S. Akilesh, T. J. Sproule, G. J. Christianson, H. Al Khabbaz, A. C. Brown, L. G. Presta, Y. G. Meng and D. C. Roopenian (2006). “Enhanced half-life of genetically engineered human IgG1 antibodies in a humanized FcRn mouse model: potential application in humorally mediated autoimmune disease.” Int Immunol 18(12): 1759-1769.
Shealy, D. J., A. Cai, K. Staquet, A. Baker, E. R. Lacy, L. Johns, O. Vafa, G. Gunn, 3rd, S. Tam, S. Sague, D. Wang, M. Brigham-Burke, P. Dalmonte, E. Emmell, B. Pikounis, P. J. Bugelski, H. Zhou, B. J. Scallon and J. Giles-Komar (2010). “Characterization of golimumab, a human monoclonal antibody specific for human tumor necrosis factor alpha.” MAbs 2(4): 428-439.
Shields, R. L., A. K. Namenuk, K. Hong, Y. G. Meng, J. Rae, J. Briggs, D. Xie, J. Lai, A. Stadlen, B. Li, J. A. Fox and L. G. Presta (2001). “High resolution mapping of the binding site on human IgG1 for Fc gamma RI, Fc gamma RII, Fc gamma RIII, and FcRn and design of IgG1 variants with improved binding to the Fc gamma R.” J Biol Chem 276(9): 6591-6604.
Smith, L. B., M. C. Leo, C. Anderson, T. J. Wright, K. B. Weymann and L. J. Wood (2014). “The role of IL-1beta and TNF-alpha signaling in the genesis of cancer treatment related symptoms (CTRS): a study using cytokine receptor-deficient mice.” Brain Behav Immun 38: 66-76.
Sockolosky, J. T., M. R. Tiffany and F. C. Szoka (2012). “Engineering neonatal Fc receptor-mediated recycling and transcytosis in recombinant proteins by short terminal peptide extensions.” Proc Natl Acad Sci USA 109(40): 16095-16100.
Tam, S. H., S. G. McCarthy, A. A. Armstrong, S. Somani, S. J. Wu, X. Liu, A. Gervais, R. Ernst, D. Saro, R. Decker, J. Luo, G. L. Gilliland, M. L. Chiu and B. J. Scallon (2017). “Functional, Biophysical, and Structural Characterization of Human IgG1 and IgG4 Fc Variants with Ablated Immune Functionality.” Antibodies (Basel) 6(3).
Tang, Q., L. Hao, Y. Peng, Y. Zheng, K. Sun, F. Cai, C. Liu and Q. Liao (2015). “RNAi Silencing of IL-1beta and TNF-alpha in the Treatment of Post-traumatic Arthritis in Rabbits.” Chem Biol Drug Des 86(6): 1466-1470.
Taylor, P. C. (2010). “Pharmacology of TNF blockade in rheumatoid arthritis and other chronic inflammatory diseases.” Curr Opin Pharmacol 10(3): 308-315.
Turner, M. D., B. Nedjai, T. Hurst and D. J. Pennington (2014). “Cytokines and chemokines: At the crossroads of cell signalling and inflammatory disease.” Biochim Biophys Acta 1843(11): 2563-2582.
Vafa, O., G. L. Gilliland, R. J. Brerski, B. Strake, T. Wilkinson, E. R. Lacy, B. Scallon, A. Teplyakov, T. J. Malia and W. R. Strohl (2014). “An engineered Fc variant of an IgG eliminates all immune effector functions via structural perturbations.” Methods 65(1): 114-126.
Worn, A. and A. Pluckthun (2001). “Stability engineering of antibody single-chain Fv fragments.” J Mol Biol 305(5): 989-1010.
Wu, S., T. Kanda, S. Nakamoto, X. Jiang, M. Nakamura, R. Sasaki, Y. Haga, H. Shirasawa and O. Yokosuka (2016). “Cooperative effects of hepatitis B virus and TNF may play important roles in the activation of metabolic pathways through the activation of NF-kappaB.” Int J Mol Med 38(2): 475-481.
Xiao, G. and L. S. Gan (2013). “Receptor-mediated endocytosis and brain delivery of therapeutic biologics.” Int J Cell Biol 2013: 703545.
Xu, D., M. L. Alegre, S. S. Varga, A. L. Rothermel, A. M. Collins, V. L. Pulito, L. S. Hanna, K. P. Dolan, P. W. Parren, J. A. Bluestone, L. K. Jolliffe and R. A. Zivin (2000). “In vitro characterization of five humanized OKT3 effector function variant antibodies.” Cell Immunol 200(1): 16-26.
Xu, M., J. Lv, P. Wang, Y. Liao, Y. Li, W. Zhao, J. Zen, Z. Dong, Z. Guo, X. Bo, M. Liu, L. Zhang, G. Hu and Y. Chen (2019). “Vascular endothelial Cdc42 deficiency delays skin wound-healing processes by increasing IL-1beta and TNF-alpha expression.” Am J Transl Res 11(1): 257-268.
Yokose, K., S. Sato, T. Asano, M. Yashiro, H. Kobayashi, H. Watanabe, E. Suzuki, C. Sato, H. Kozuru, H. Yatsuhashi and K. Migita (2018). “TNF-alpha potentiates uric acid-induced interleukin-1beta (IL-1beta) secretion in human neutrophils.” Mod Rheumatol 28(3): 513-517.
Zalevsky, J., A. K. Chamberlain, H. M. Horton, S. Karki, I. W. Leung, T. J. Sproule, G. A. Lazar, D. C. Roopenian and J. R. Desjarlais (2010). “Enhanced antibody half-life improves in vivo activity.” Nat Biotechnol 28(2): 157-159.

All references cited herein, including the entire disclosures of these references/publications, and all disclosures, disclosure application publications and books referred to herein, are hereby incorporated by reference in their entirety into the subject application.

We claim:

1. A bispecific antibody or antigen-binding fragment with dual binding specificity to both tumor necrosis factor alpha (TNFα) and interleukin 1β (IL-1β), comprising:
a) a heavy chain with binding specificity to TNFα and a light chain with binding specificity to TNFα; and
b) a heavy chain with binding specificity to IL-1β and a light chain with binding specificity to IL-1β.

2. The bispecific antibody or antigen-binding fragment of claim 1, wherein the bispecific antibody is capable of neutralizing, reducing, or interfering with an activity of TNFα and an activity of IL-1β.

3. The bispecific antibody or antigen-binding fragment of claim 1, wherein
(i) the heavy chain with binding specificity to TNFα comprises a heavy chain variable domain with an amino acid sequence having at least 80% sequence identity to an amino acid sequence set forth as any one of SEQ ID NO: 1 to SEQ ID NO: 9;
(ii) the light chain with binding specificity to TNFα comprises a light chain variable domain with an amino acid sequence having at least 80% sequence identity to an amino acid sequence set forth as any one of SEQ ID NO: 10 to SEQ ID NO: 12;
(iii) the heavy chain with binding specificity to TNFα comprises a human IgG heavy chain with an amino acid sequence having at least 80% sequence identity to an amino acid sequence set forth as any one of SEQ ID NO: 13 to SEQ ID NO: 27;
(iv) the light chain with binding specificity to TNFα comprises a human IgG light chain with an amino acid sequence having at least 80% sequence identity to an amino acid sequence set forth as any one of SEQ ID NO: 28 to SEQ ID NO: 30;
(v) the heavy chain with binding specificity to IL1β comprises a heavy chain variable domain with an amino acid sequence having at least 80% sequence identity to an amino acid sequence set forth as any one of SEQ ID NO: 31 to SEQ ID NO: 33;
(vi) the light chain with binding specificity to IL1β comprises a light chain variable domain with an amino acid sequence having at least 80% sequence identity to an amino acid sequence set forth as any one of SEQ ID NO: 34 to SEQ ID NO: 36;
(vii) the heavy chain with binding specificity to IL1β comprises a human IgG heavy chain with an amino acid sequence having at least 80% sequence identity to an amino acid sequence set forth as any one of SEQ ID NO: 37 to SEQ ID NO: 43;
(viii) the light chain with binding specificity to IL1β comprises a human IgG light chain with an amino acid sequence having at least 80% sequence identity to an amino acid sequence set forth as any one of SEQ ID NO: 44 to SEQ ID NO: 46;
(ix) the heavy chain and light chain with binding specificity to TNFα comprises combinations of heavy chain variable domains and light chain variable domains with different IgG Fc listed in Table 2;
(x) the heavy chain and light chain with binding specificity to IL1β comprises combinations of heavy chain variable domains and light chain variable domains with different IgG Fc listed in Table 3; or
(xi) the heavy chain and light chain with binding specificity to TNFα and the heavy chain and light chain with binding specificity to IL1β comprises combinations of heavy chains and light chains listed in Table 4.

4.-13. (canceled)

14. The bispecific antibody or antigen-binding fragment of claim 1, wherein
(i) the heavy chain and the light chain with binding specificity to TNFα is an IgG₁, IgG₂, IgG₃ or IgG₄ isotype, and the heavy chain and the light chain with binding specificity to IL-1β is an IgG₁, IgG₂, IgG₃ or IgG₄ isotype;
(ii) the heavy chain with binding specificity to TNFα and/or the heavy chain with binding specificity to IL-1β has one or more F_{c} mutations that extends the half-life of the bispecific antibody when compared to a wild-type antibody without the mutations;
(iii) the C_{H2} and C_{H3} domains of the heavy chain with binding specificity to TNFα and/or the heavy chain with binding specificity to IL-1β has any one set of mutations selected from M252Y/S254T/T256E, M428L/N434S, T250Q/M428L, N434A and T307A/E380A/N434A when compared to a wild-type antibody without the mutations, according to the EU Index residue numbering;
(iv) the heavy chain with binding specificity to TNFα and/or the heavy chain with binding specificity to IL-1β has one or more F_{c} and hinge mutations that enhance the resistance of the bispecific antibody to proteolytic degradation by a protease that cleaves a wild-type antibody without the mutations between or at residues 222-237, according to the EU Index residue numbering;
(v) the hinge region of the heavy chain with binding specificity to TNFα and/or the heavy chain with binding specificity to IL-1β comprises E233P/L234A/L235A mutations with G236 deleted when compared to a wild-type antibody without the mutations, with residue numbering according to the EU Index residue numbering;
(vi) the heavy chain with binding specificity to TNFα and/or the heavy chain with binding specificity to IL-1β has one or more F_{c} mutations that that reduce or eliminate the effector functions of the antibody compared to a wild-type antibody without the mutations;
(vii) the C_{H2} and C_{H3} domains of the heavy chain with binding specificity to TNFα and/or the heavy chain with binding specificity to IL-1β has L234A, L235A, M428L and N434S F_{c} mutations that extend the half-life and reduce the effector functions of the antibody, with residue numbering according to the EU Index, compared to a wild-type antibody;
(viii) the C_{H2} and C_{H3} domains of the heavy chain with binding specificity to TNFα and/or the heavy chain with binding specificity to IL-1β has E233P, L234A, L235A, M428L and N434S F_{c} mutations with G236 deleted that extend the half-life, reduce the effector functions and enhance the resistance of the bispecific antibody to proteolytic degradation by a protease, with residue numbering according to the EU Index, compared to a wild-type antibody; or
(ix) the C_{H2} and C_{H3} domains of the heavy chain with binding specificity to TNFα and/or the heavy chain with binding specificity to IL-1β has F_{c} mutations which can facilitate heavy chain heterodimerization when compared to a wild-type antibody without the mutations, wherein the mutations comprise an F405L mutation and/or a K409R mutation, with residue numbering according to the EU Index.

15.-22. (canceled)

23. The bispecific antibody or antigen-binding fragment of claim 1, wherein the bispecific antibody
(i) is capable of blocking the binding of TNFα and/or IL-1β to their respective receptors;
(ii) neutralizes, reduces, or interferes the functional activity of TNFα and/or IL-1β to their receptors;
(iii) neutralizes the TNFα and/or IL-1β-driven reporter gene activation in reporter gene assays;
(iv) neutralizes the TNFα-driven cytotoxicity to a murine fibrosarcoma WEHI cell line in a WEHI cell-based cytotoxicity assay;
(v) neutralizes the IL-1β-driven IL6 release from the activation of human lung fibroblast cell line MRC-5;
(vi) neutralizes the TNFα and/or IL-1β driven inflammation in a Collagen antibody induced arthritis (CAIA) mouse model; and/or
(vii) neutralizes the TNFα and/or IL-1β driven knee joint inflammation in a human TNFα and/or IL-1β induced knee joint inflammation mouse model.

24.-29. (canceled)

30. A polynucleotide encoding the bispecific antibody or antigen-binding fragment of claim 1.

31. A vector comprising the polynucleotides of claim 30.

32. (canceled)

33. A host cell comprising the vector of claim 31.

34. A method of producing engineered anti-TNFα and anti-IL-1β IgG antibodies as parental antibodies, comprising culturing the host cell of claim 33 in conditions wherein the engineered anti-TNFα and anti-IL-1β IgG antibodies are expressed, and isolating the engineered anti-TNFα and anti-IL-1β IgG antibodies.

35. A method of generation of the anti-TNFα and IL-1β bispecific antibody of claim 1 from two parental antibodies or two separate culture supernatants by controlled F_{ab} arm exchange.

36.-39. (canceled)

40. A pharmaceutical composition comprising the bispecific antibody of claim 1.

41. A method for treating an TNFα and/or IL-1β mediated disease or disorder in a subject in need thereof, comprising administering to the subject an effective amount of the anti-TNFα and IL-1β bispecific antibody of claim 1.

42. The method according to claim 41, wherein the TNFα and/or IL-1β mediated disease or disorder is selected from an auto-immune disease, an inflammatory disease, a diabetes related disease, a skin disease, an eye disease, a neurological disease, a cancer, a chronic hepatitis B infection, and atrophic thyroiditis.

43. The method of claim 42, wherein the auto-immune or inflammatory disease is selected from the group consisting of rheumatoid arthritis, systemic lupus erythematosus, osteoarthritis, ankylosing spondylitis, Behcet's Disease, gout, psoriatic arthritis, multiple sclerosis, Crohn's colitis, small intestine enteropathy and inflammatory bowel disease.

44. (canceled)

45. The method of claim 42, wherein the diabetes related disease is selected from the group consisting of Type II diabetes mellitus, proliferative diabetic retinopathy, diabetic neuropathy, and fulminant Type 1 diabetes.

46. (canceled)

47. The method of claim 42, wherein the skin disease is selected from the group consisting of wound healing, leprosy, and decubitus ulcer.

48. (canceled)

49. The method of claim 42, wherein the eye disease is selected from the group consisting of age-related macular degeneration, retinal vasculitis, and non-infectious posterior uveitis.

50. (canceled)

51. The method of claim 42, wherein the neurological disease is selected from the group consisting of Parkinson's disease, polyneuropathy, sensory peripheral neuropathy, alcoholic neuropathy and sciatic neuropathy.

52. (canceled)

53. The method of claim 42, wherein the cancer is selected from the group consisting of multiple myeloma, non-small cell lung cancer, acute myeloid leukemia, female breast cancer, pancreatic cancer, colorectal cancer, and peritoneum cancer.

54. (canceled)

55. The method of claim 41, wherein said administering is selected from subcutaneous, intravenous, intramuscular, oral, rectal, systemic, and local.

56.-60. (canceled)

61. The method of claim 41, further comprising administering a second agent to the subject in need of treatment.

62. The method of claim 61, wherein the second agent is a standard of care therapy selected from the group consisting of corticosteroids, anti-cancer drugs, immunomodulatory drugs, and cytokine therapy drugs.

63. (canceled)


SEQUENCE LISTING (62 sequences)

SEQ ID NO: 1 - protein, 121 aa, Artificial Sequence [sequence omitted]

SEQ ID NO: 2 - protein, 121 aa, Artificial Sequence
  Heavy chain variable domain ADA-H1 of anti-TNF alpha antibody
EVQLVESGGVVVQPGGSLRLSCAASGFTFDDYAMHWVRQAPGKGLEWVSAITWNSGHIDYADSVKGRFTISRDNSKNSLYLQMNSLRTEDTALYYCAKVSYLSTASSLDYWGQGTLVTVSS

SEQ ID NO: 3 - protein, 121 aa, Artificial Sequence
  Heavy chain variable domain ADA-H1X of anti-TNF alpha antibody
EVQLVESGGVVVQPGGSLRLSCAASGFDFADYAMHWVRQAPGKGLEWVSAITWNGGHTDYADSVKGRFTISRDNSKNSLYLQMNSLRTEDTALYYCAKVSYLSTASSLDYWGQGTLVTVSS

SEQ ID NO: 4 - protein, 121 aa, Artificial Sequence
  Heavy chain variable domain ADA-H2 of anti-TNF alpha antibody
EVQLVESGGGLVQPGGSLRLSCAASGFTFDDYAMHWVRQAPGKGLEWVSAITWNSGHIDYADSVKGRFTISRDNSKNTLYLQMNSLRAEDTAVYYCAKVSYLSTASSLDYWGQGTLVTVSS

SEQ ID NO: 5 - protein, 121 aa, Artificial Sequence
  Heavy chain variable domain ADA-H2X of anti-TNF alpha antibody
EVQLVESGGGLVQPGGSLRLSCAASGFDFADYAMHWVRQAPGKGLEWVSAITWNGGHTDYADSVKGRFTISRDNSKNTLYLQMNSLRAEDTAVYYCAKVSYLSTASSLDYWGQGTLVTVSS

SEQ ID NO: 6 - protein, 121 aa, Artificial Sequence
  Heavy chain variable domain ADA-H3 of anti-TNF alpha antibody
EVQLVESGGGLVQPGGSLRLSCAASGFTFDDYAMHWVRQAPGKGLVWVSAITWNSGHIDYADSVKGRFTISRDNAKNTLYLQMNSLRAEDTAVYYCAKVSYLSTASSLDYWGQGTLVTVSS

SEQ ID NO: 7 - protein, 121 aa, Artificial Sequence
  Heavy chain variable domain ADA-H3X of anti-TNF alpha antibody
EVQLVESGGGLVQPGGSLRLSCAASGFDFADYAMHWVRQAPGKGLVWVSAITWNGGHTDYADSVKGRFTISRDNAKNTLYLQMNSLRAEDTAVYYCAKVSYLSTASSLDYWGQGTLVTVSS

SEQ ID NO: 8 - protein, 121 aa, Artificial Sequence
  Heavy chain variable domain ADA-H4 of anti-TNF alpha antibody
QVQLVESGGGVVQPGGSLRLSCAASGFTFDDYAMHWVRQAPGKGLEWVSAITWNSGHIDYADSVKGRFTISRDNSKNTLYLQMNSLRAEDTAVYYCAKVSYLSTASSLDYWGQGTLVTVSS

SEQ ID NO: 9 - protein, 121 aa, Artificial Sequence
  Heavy chain variable domain ADA-H4X of anti-TNF alpha antibody
QVQLVESGGGVVQPGGSLRLSCAASGFDFADYAMHWVRQAPGKGLEWVSAITWNGGHTDYADSVKGRFTISRDNSKNTLYLQMNSLRAEDTAVYYCAKVSYLSTASSLDYWGQGTLVTVSS

SEQ ID NO: 10 - protein, 107 aa, Artificial Sequence [sequence omitted]

SEQ ID NO: 11 - protein, 107 aa, Artificial Sequence
  Light chain variable domain ADA-L1 of anti-TNF alpha antibody
EIVMTQSPATLSVSPGERATLSCRASQGIRNYLAWYQQKPGQAPRLLIYAASTLQSGIPARFSGSGSGTEFTLTISSLQSEDFAVYYCQRYNRAPYTFGQGTKVEIK

SEQ ID NO: 12 - protein, 107 aa, Artificial Sequence
  Light chain variable domain ADA-L2 of anti-TNF alpha antibody
DIVMTQSPDSLAVSLGERATINCRASQGIRNYLAWYQQKPGQAPKLLIYAASTLQSGVPDRFSGSGSGTDFTLTISSLQAEDVAVYYCQRYNRAPYTFGQGTKVEIK

SEQ ID NO: 13 - protein, 451 aa, Artificial Sequence
  anti-TNF alpha antibody heavy chain EAC33
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 14 - protein, 451 aa, Artificial Sequence
  anti-TNF alpha antibody heavy chain EAC119
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 15 - protein, 451 aa, Artificial Sequence
  anti-TNF alpha antibody heavy chain EAC129
EVQLVESGGVVVQPGGSLRLSCAASGFTFDDYAMHWVRQAPGKGLEWVSAITWNSGHIDYADSVKGRFTISRDNSKNSLYLQMNSLRTEDTALYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 16 - protein, 451 aa, Artificial Sequence
  anti-TNF alpha antibody heavy chain EAC130
EVQLVESGGVVVQPGGSLRLSCAASGFDFADYAMHWVRQAPGKGLEWVSAITWNGGHTDYADSVKGRFTISRDNSKNSLYLQMNSLRTEDTALYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 17 - protein, 451 aa, Artificial Sequence
  anti-TNF alpha antibody heavy chain EAC131
EVQLVESGGGLVQPGGSLRLSCAASGFTFDDYAMHWVRQAPGKGLEWVSAITWNSGHIDYADSVKGRFTISRDNSKNTLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 18 - protein, 451 aa, Artificial Sequence
  anti-TNF alpha antibody heavy chain EAC132
EVQLVESGGGLVQPGGSLRLSCAASGFDFADYAMHWVRQAPGKGLEWVSAITWNGGHTDYADSVKGRFTISRDNSKNTLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 19 - protein, 451 aa, Artificial Sequence
  anti-TNF alpha antibody heavy chain EAC133
EVQLVESGGGLVQPGGSLRLSCAASGFTFDDYAMHWVRQAPGKGLVWVSAITWNSGHIDYADSVKGRFTISRDNAKNTLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 20 - protein, 451 aa, Artificial Sequence
  anti-TNF alpha antibody heavy chain EAC134
EVQLVESGGGLVQPGGSLRLSCAASGFDFADYAMHWVRQAPGKGLVWVSAITWNGGHTDYADSVKGRFTISRDNAKNTLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 21 - protein, 451 aa, Artificial Sequence
  anti-TNF alpha antibody heavy chain EAC135
QVQLVESGGGVVQPGGSLRLSCAASGFTFDDYAMHWVRQAPGKGLEWVSAITWNSGHIDYADSVKGRFTISRDNSKNTLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 22 - protein, 451 aa, Artificial Sequence
  anti-TNF alpha antibody heavy chain EAC136
QVQLVESGGGVVQPGGSLRLSCAASGFDFADYAMHWVRQAPGKGLEWVSAITWNGGHTDYADSVKGRFTISRDNSKNTLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 23 - protein, 450 aa, Artificial Sequence
  anti-TNF alpha antibody heavy chain EAC144
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 24 - protein, 450 aa, Artificial Sequence
  anti-TNF alpha antibody heavy chain EAC166
EVQLVESGGVVVQPGGSLRLSCAASGFDFADYAMHWVRQAPGKGLEWVSAITWNGGHTDYADSVKGRFTISRDNSKNSLYLQMNSLRTEDTALYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 25 - protein, 450 aa, Artificial Sequence
  anti-TNF alpha antibody heavy chain EAC167
EVQLVESGGGLVQPGGSLRLSCAASGFDFADYAMHWVRQAPGKGLEWVSAITWNGGHTDYADSVKGRFTISRDNSKNTLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 26 - protein, 450 aa, Artificial Sequence
  anti-TNF alpha antibody heavy chain EAC168
EVQLVESGGGLVQPGGSLRLSCAASGFDFADYAMHWVRQAPGKGLVWVSAITWNGGHTDYADSVKGRFTISRDNAKNTLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 27 - protein, 450 aa, Artificial Sequence
  anti-TNF alpha antibody heavy chain EAC169
QVQLVESGGGVVQPGGSLRLSCAASGFDFADYAMHWVRQAPGKGLEWVSAITWNGGHTDYADSVKGRFTISRDNSKNTLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 28 - protein, 214 aa, Artificial Sequence
  anti-TNF alpha antibody light chain EAC34
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 214 aa, Artificial Sequence
  anti-TNF alpha antibody light chain EAC127
EIVMTQSPATLSVSPGERATLSCRASQGIRNYLAWYQQKPGQAPRLLIYAASTLQSGIPARFSGSGSGTEFTLTISSLQSEDFAVYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 214 aa, Artificial Sequence
  anti-TNF alpha antibody light chain EAC128
DIVMTQSPDSLAVSLGERATINCRASQGIRNYLAWYQQKPGQAPKLLIYAASTLQSGVPDRFSGSGSGTDFTLTISSLQAEDVAVYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 120 aa, Artificial Sequence
  Heavy chain variable domain Ab5H3 of anti-IL1 beta antibody
QVQLVESGGGVVQPGRSLRLSCAFSGFSLSTSGMGVGWIRQAPGKGLEWVAHIWWDGDESYADSVKGRFTISKDNSKNTVYLQMNSLRAEDTAVYFCARNRYDPPWFVDWGQGTLVTVSS

SEQ ID NO: 32 - protein, 121 aa, Artificial Sequence
  Heavy chain variable domain Ab8H1 of anti-IL1 beta antibody
EVQLVESGGGLVQPGGSLRLSCAASGFTFSSFGMHWVRQAPGKGLEWVAYISIGSYTVHYADSVKGRFTISRDNAKNSLYLQMNSLRDEDTAVYYCVRDDYDVTDYTMDYWGQGTTVTVSS

SEQ ID NO: 33 - protein, 120 aa, Artificial Sequence
  Heavy chain variable domain Ab9H1 of anti-IL1 beta antibody
QVTLKESGPALVKPTQTLTLTCTFSGFSLSTSGMGVSWIRQPPGKGLEWLAHIYWDDDKYYSPSLKSRLTITKDTSKNQVVLTMTNMDPVDTATYYCARGSYDPSPFDYWGQGTTVTVSS

SEQ ID NO: 34 - protein, 107 aa, Artificial Sequence [sequence omitted]

SEQ ID NO: 35 - protein, 112 aa, Artificial Sequence
  Light chain variable domain Ab8L3 of anti-IL1 beta antibody
DIVMTQTPLSLPVTPGEPASISCKSSQSLLNSRTRKNYLAWYLQKPGQSPQLLIYWASTRESGVPDRFSGSGSGTDFTLKISRVEAEDVGVYYCKQTYNFPTFGQGTKLEIK

SEQ ID NO: 36 - protein, 107 aa, Artificial Sequence
  Light chain variable domain Ab9L1 of anti-IL1 beta antibody
DIQMTQSPSSLSASVGDRVTITCRPSRDITNYLNWYQQKPGKTLKLLIYHTSRLHSGVPSRFSGSGSGTDYTLTISSLQPEDFATYFCQQSKSVPWTFGGGTKVEIK

SEQ ID NO: 37 - protein, 450 aa, Artificial Sequence
  anti-IL1 beta antibody heavy chain EAC53
QVQLVESGGGVVQPGRSLRLSCAFSGFSLSTSGMGVGWIRQAPGKGLEWVAHIWWDGDESYADSVKGRFTISKDNSKNTVYLQMNSLRAEDTAVYFCARNRYDPPWFVDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 38 - protein, 451 aa, Artificial Sequence
  anti-IL1 beta antibody heavy chain EAC73
EVQLVESGGGLVQPGGSLRLSCAASGFTFSSFGMHWVRQAPGKGLEWVAYISIGSYTVHYADSVKGRFTISRDNAKNSLYLQMNSLRDEDTAVYYCVRDDYDVTDYTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 39 - protein, 450 aa, Artificial Sequence
  anti-IL1 beta antibody heavy chain EAC80
QVTLKESGPALVKPTQTLTLTCTFSGFSLSTSGMGVSWIRQPPGKGLEWLAHIYWDDDKYYSPSLKSRLTITKDTSKNQVVLTMTNMDPVDTATYYCARGSYDPSPFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 40 - protein, 450 aa, Artificial Sequence
  anti-IL1 beta antibody heavy chain EAC120
QVQLVESGGGVVQPGRSLRLSCAFSGFSLSTSGMGVGWIRQAPGKGLEWVAHIWWDGDESYADSVKGRFTISKDNSKNTVYLQMNSLRAEDTAVYFCARNRYDPPWFVDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 41 - protein, 451 aa, Artificial Sequence
  anti-IL1 beta antibody heavy chain EAC121
EVQLVESGGGLVQPGGSLRLSCAASGFTFSSFGMHWVRQAPGKGLEWVAYISIGSYTVHYADSVKGRFTISRDNAKNSLYLQMNSLRDEDTAVYYCVRDDYDVTDYTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 42 - protein, 450 aa, Artificial Sequence
  anti-IL1 beta antibody heavy chain EAC145
EVQLVESGGGLVQPGGSLRLSCAASGFTFSSFGMHWVRQAPGKGLEWVAYISIGSYTVHYADSVKGRFTISRDNAKNSLYLQMNSLRDEDTAVYYCVRDDYDVTDYTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 43 - protein, 449 aa, Artificial Sequence
  anti-IL1 beta antibody heavy chain EAC161
QVTLKESGPALVKPTQTLTLTCTFSGFSLSTSGMGVSWIRQPPGKGLEWLAHIYWDDDKYYSPSLKSRLTITKDTSKNQVVLTMTNMDPVDTATYYCARGSYDPSPFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 44 - protein, 214 aa, Artificial Sequence
  anti-IL1 beta antibody light chain EAC32
DIQMTQSTSSLSASVGDRVTITCRASQDISNYLSWYQQKPGKAVKLLIYYTSKLHSGVPSRFSGSGSGTDYTLTISSLQQEDFATYFCLQGKMLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 219 aa, Artificial Sequence
  anti-IL1 beta antibody light chain EAC78
DIVMTQTPLSLPVTPGEPASISCKSSQSLLNSRTRKNYLAWYLQKPGQSPQLLIYWASTRESGVPDRFSGSGSGTDFTLKISRVEAEDVGVYYCKQTYNFPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 214 aa, Artificial Sequence
  anti-IL1 beta antibody light chain EAC83
DIQMTQSPSSLSASVGDRVTITCRPSRDITNYLNWYQQKPGKTLKLLIYHTSRLHSGVPSRFSGSGSGTDYTLTISSLQPEDFATYFCQQSKSVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 234 aa, Artificial Sequence
  IgG1 Fc
KVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 230 aa, Artificial Sequence
  IgG2 Fc
TVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 49 - protein, 236 aa, Artificial Sequence
  IgG3 Fc
RVELKTPLGDTTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 231 aa, Artificial Sequence
  IgG4 Fc
RVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 51 - protein, 234 aa, Artificial Sequence
  IgG1 Fc with M252Y/S254T/T256E mutations
KVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 234 aa, Artificial Sequence
  IgG1 Fc with M428L/N434S mutations
KVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 53 - protein, 234 aa, Artificial Sequence
  IgG1 Fc with T250Q/M428L mutations
KVEPKSCDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 54 - protein, 234 aa, Artificial Sequence
  IgG1 Fc with N434A mutations
KVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 55 - protein, 234 aa, Artificial Sequence
  IgG1 Fc with T307A/E380A/N434A mutations
KVEPKSCDKTHTCPPCPAPELLGGPSVFLFPPKPKDTLMISRTPEVTCVVVDVSHEDPEVKFNWYVDGVEVHNAKTKPREEQYNSTYRVVSVLAVLHQDWLNGKEYKCKVSNKALPAPIEKTISKAKGQPREPQVYTLPPSRDELTKNQVSLTCLVKGFYPSDIAVAWESNGQPENNYKTTPPVLDSDGSFFLYSKLTVDKSRWQQGNVFSCSVMHEALHAHYTQKSLSLSPGK

SEQ ID NO: 56 - protein, 233 aa, Artificial Sequence
  IgG1 Fc with E233P/L234A/L235A mutations and G236 deleted
KVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 234 aa, Artificial Sequence
  IgG1 Fc with F405L mutation
KVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 58 - protein, 234 aa, Artificial Sequence
  IgG1 Fc with K409R mutation
KVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 59 - DNA, 1353 bp, Artificial Sequence
  Polynucleotide sequence encoding EAC33
gaggtgcagctggtggagagcggcggaggactggtgcagcccggtagatctttaagactgagctgtgccgccagcggcttcacattcgacgactacgccatgcactgggtgagacaagctcccggtaaaggtttagaatgggtgagcgccatcacttggaacagcggccacatcgactacgccgacagcgtggagggtcgtttcaccatctctcgtgacaacgccaagaactctttatatttacagatgaactctttaagagccgaggacaccgccgtgtactactgcgccaaggtgagctatttaagcaccgccagctctttagactactggggccaaggtactttagtgaccgtgagcagcgccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttgctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 60 - DNA, 642 bp, Artificial Sequence
  Polynucleotide sequence encoding EAC34
gacatccagatgacccagagccctagctctttaagcgctagcgtgggcgatcgtgtgaccatcacttgtcgtgccagccaaggtattcgtaactatttagcttggtaccagcagaagcccggcaaggcccccaagctgctgatctacgccgccagcactttacagagcggagtgcctagcagatttagcggcagcggtagcggcaccgatttcactttaaccatcagctctttacagcccgaagacgtggccacctactactgccagaggtacaatcgtgccccctacacctttggccaaggtaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 61 - DNA, 1350 bp, Artificial Sequence
  Polynucleotide sequence encoding EAC53
caagttcagctggtggagagcggaggaggcgtggtgcagcccggtagatctttaaggctgagctgcgccttcagcggcttctctttaagcaccagcggaatgggcgtgggctggatcagacaagctcccggaaagggtttagagtgggtggcccacatctggtgggacggcgacgagagctacgccgacagcgtgaagggtcgtttcaccatcagcaaggacaactccaagaacaccgtgtatttacagatgaactctttaagggccgaggacaccgccgtgtacttctgcgctcgtaatcgttacgaccccccttggtttgtggactggggccaaggtactttagtgacagtgagcagcgccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcagactcaccgtggacaagagcaggtggcagcaggggaacgtcttctcatgctccgtgatgcatgaggctctgcacaaccactacacgcagaagagcctctccctgtctccgggtaaa

SEQ ID NO: 62 - DNA, 642 bp, Artificial Sequence
  Polynucleotide sequence encoding EAC32
gacatccagatgacccagagcacatcctctttatccgccagcgtgggcgacagagtgaccatcacttgtcgtgccagccaagatatcagcaactatttaagctggtaccagcagaagcccggcaaggccgtgaagctgctgatctactacaccagcaagctgcacagcggcgtgcctagcagattcagcggcagcggaagcggcaccgactacactttaaccatcagctctttacagcaagaagacttcgccacctacttctgtttacaaggtaagatgctgccttggaccttcggccaaggtaccaagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt